(19) United States
(12) Patent Application Publication
(10) Pub. No.: US 2024/0124464 A1
(43) Pub. Date: Apr. 18, 2024

(54) SUBSTITUTED FUSED BICYCLIC COMPOUND AS KINASE INHIBITOR AND USE THEREOF

(71) Applicant: IMPACT THERAPEUTICS (SHANGHAI), INC, Shanghai (CN)

(72) Inventors: Sui Xiong CAI, Shanghai (CN); Ye Edward TIAN, Shanghai (CN); Xiaozhu WANG, Jiangsu (CN)

(21) Appl. No.: 18/255,476

(22) PCT Filed: Nov. 29, 2021
(86) PCT No.: PCT/CN2021/134071

§ 371 Date: Jun. 1, 2023

(30) Foreign Application Priority Data
Dec. 2, 2020 (CN) 202011414870.5

(51) Int. Cl. C07D 487/04; A61K 45/06; A61P 35/00; C07D 471/04; C07D 498/04

(52) U.S. Cl. CPC C07D 487/04; A61K 45/06; A61P 35/00; C07D 471/04; C07D 498/04

(57) ABSTRACT

Provided in the present invention are a substituted fused bicyclic compound as a kinase inhibitor and the use thereof. The substituted fused bicyclic compound has a structure as represented by formula I below, wherein a ring A, R₀, B₁-B₃, D₁-D₃, R₇ and R₈ are defined herein. The compound of formula I is an NUAK1/2 inhibitor. Therefore, the compound of the present invention can be used for treating and preventing NUAK1/2-mediated diseases, disorders and conditions, such as cancer, and used in the preparation of drugs for treating and preventing NUAK1/2-mediated diseases, disorders and conditions.

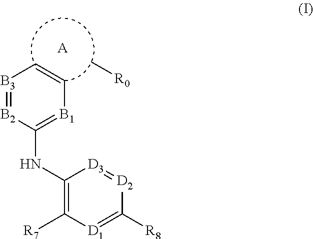

### TECHNICAL FIELD

This disclosure is in the field of medicinal chemistry. The disclosure relates to substituted fused bicyclic compounds, and the use of these compounds as therapeutically effective kinase inhibitors and anticancer drugs.

### BACKGROUND

AMPK (Adenosine 5′-monophosphate (AMP)-activated protein kinase or AMP-dependent protein kinase), a serine/threonine protein kinase, has been reported to play a critical role in cell energy homeostasis, glucose and cholesterol metabolism as well as cell proliferation (Tiziana et al., 2015; Vincent et al., 2015). The activity of AMPK is dependent on the intracellular AMP/ATP ratio (Sanz P et al., 2008). When intracellular ATP concentration decreases, elevated 5′-AMP activates AMPK. AMPK has a heterotrimer structure of one catalytic subunit (α) and two regulatory subunits (β and γ). Up to date, 12 AMPK-related kinases (ARKs) have been identified based on the similarity to AMPK α subunit, including BRSK1, BRSK2, NUAK1, NUAK2, QIK, QSK, SIK, MARK1, MARK2, MARK3, MARK4, and MELK. One of the pieces of evidence connecting AMPK to cancer is that all ARKs except MELK are substrates of liver kinase B1 (LKB1) whereas LKB1 is a multitasking tumor suppressor kinase. They are activated by LKB1 through phosphorylation at threonine equivalent to position 172 of AMPK catalytic subunit (Sun et al., 2013).

NUAK1 (also known as NUAK family SNF1-like kinase 1 or AMPK related protein kinase 5 (ARKS)) and NUAK2 (also known as NUAK family SNF1-like kinase 2 or SNF1/A1VIP kinase-related kinase (SNARK)) are members of ARK family and share high degree of homology in structure. They are similar to the α catalytic subunit of AMPK structurally, and contain a ubiquitin-association domain in the C-terminal (Bright et al. 2009), which is essential for the LKB1-dependent phosphorylation and activation. The homology of protein sequences of human NUAK1 and NUAK2 is 55.0% (Suzuki et al. 2003).

NUAK1, discovered by Suzuki et al in 2003, contains 661 amino acids with a molecular weight of 74 kD. NUAK1 has been found in organs such as the heart, kidney, liver, brain, and skeletal muscle and is over expressed in a variety of cancer cells such as multiple myeloma cells. NUAK1 has been shown to be associated with the AKT signaling pathway, especially with IGF-induced cell migration and invasion (Kusakai et al., 2004). NUAK1 is also involved in tumor generation and survival. It inhibits tumor cell apoptosis induced by glucose starvation, cytokines and TNFα (Atsushi et al., 2003), and involved in AKT-dependent tumor cell proliferation and metastasis. In summary, NUAK1 is over expressed in tumors such as breast cancer (Liu F et al., 2013), liver cancer (Cui J et al., 2013) and pancreatic cancer (Huang X et al., 2014) and plays an important role in tumor metastasis and invasion. Therefore, NUAK1 could be a target for cancer treatment.

NUAK2 was the fourth member of ARKs when it was discovered (Dmytro et al., 2012) and has functions of autophosphorylation. The human NUAK2 gene is located on chromosome 1q32.1 and codes for a 628 amino acid protein. Its molecular weight is 69 kD. NUAK2 is mainly located in the nucleus and activated in response to cellular and environmental stresses. It is part of the cellular stress responses system. Similar to AMPK, the activity of NUAK2 is regulated by AMP/ATP ratio such as in the case of glucose deprivation or chemical ATP production in cells (Bekri et al., 2014; Waise et al., 2019; Rune et al., 2009). The death receptor CD95 induces apoptosis in many tissues through TNF-α and NF-κB-mediated mechanisms and it has been reported that NUAK2 is regulated by CD95 (Zagorska et al., 2010). Studies have shown that NUAK2 is highly expressed in a variety of tumor cells and the growth and survival of melanoma are associated with NUAK2. Knocking out NUAK2 or inhibiting PI3K pathway efficiently control CDK2 expression in melanoma cells, whereas CDK2 inactivation specifically abrogated the growth of NUAK2-amplified and PTEN-deficient melanoma cells (Namiki et al., 2015), suggesting that NUAK2 has a role in melanoma tumorigenesis could be a drug target.

Like other ARKs, NUAK1 contains a highly conserved T loop in the catalytic domain, suggesting the phosphorylation of threonine by an upstream kinase. It has been shown that the phosphorylation of threonine at position 211 by LKB1 or phosphorylation of serine at position 600 by AKT activate NUAK1 (Suzuki et al. 2003b, 2006), and the activated enzymatic activity is about 10- to 20-fold higher than basal activity (Lizcano et al. 2004). The function NUAK2 is regulated similarly. NUAK2 is phosphorylated at Thr208 by LKB1, that leads to an increase in enzymatic activity by as much as 50-fold. Accordingly, NUAK2 may play a role in the physiological function of LKB1. In summary, NUAK1 and NUAK2 have important roles in cellular energy homeostasis and tumorigenesis, invasion, and metastasis. They are the potential targets to develop drugs for the treatment of cancer and metabolic diseases.

The highly selective NUAK1 inhibitors WZ4003 and HTH-01-015 have been reported by Banerjee et al. (Biochemical J. 2014, 457(1), 215). WZ4003 and HTH-01-015 inhibit the phosphorylation of MYPT1 (myosin phosphate-targeting subunit 1) by NUAK1 at Ser445. A mutation from Ala to Thr at position 195 does not affect the basal activity of NUAK1 but leads to a 50-fold resistance to these inhibitors. In a wound-healing assay using MEFs (mouse embryonic fibroblasts), treatment with WZ4003 and HTH-01-015 had similar effect as NUAK1-knockout to cell migration. Inhibition on proliferation against MEF cells of WZ4003 and HTH-01-015 was also comparable to that of shRNA knockdown of NUAK1. In a 3D invasion assay NUAK1 inhibitor WZ4003 and HTH-01-015 inhibited U2OS cell invasion similarly to NUAK1 knockout did. Therefore, WZ4003 and HTH-01-015 may be used to study the biological function of NUAK1 kinases.

WO2011156786 disclosed 6-(ethynyl)pyrido[2,3-d]pyrimidin-7(8H)-one derivatives as PAK inhibitors; US20150126508 disclosed pteridine ketone derivatives as EGFR, BLK and FLT3 inhibitors; KR2020036638 disclosed that pyrido[2,3-d]pyrimidine derivatives exhibit inhibitory ability against various kinases, particularly EGFR wild-type or mutants; US20210070731 disclosed tricyclic compounds as kinase (NUAK1, NUAK2, SIK1, CLK1 and CLK2, etc.) inhibitors; WO2021048618 and WO2021048620 disclosed 1,4-dihydrobenzo[d]pyrazolo[3,4-f][1,3]diazepine derivatives as kinase (especially LRRK2, NUAK1 and TYK2) modulators.

### SUMMARY OF THE DISCLOSURE

The disclosure provides novel substituted fused bicyclic compounds as represented in Formula I (including Formulae II and III) or pharmaceutically acceptable salts, geometric isomers, enantiomers, diastereomers, racemates, isotope-substituted derivatives, solvates, hydrates or prodrugs thereof as kinase inhibitors, especially NUAK1/2 inhibitors.

The disclosure also provides pharmaceutical compositions comprising an effective amount of the compound of Formula I (including Formulae II and III) or pharmaceutically acceptable salts, geometric isomers, enantiomers, diastereomers, racemates, isotope-substituted derivatives, solvates, hydrates or prodrugs thereof for the treatment or prevention of NUAK1/2 mediated diseases, especially cancer.

In a specific embodiment, the pharmaceutical composition may also contain one or more pharmaceutically acceptable carriers or diluents, for the treatment of cancer.

In a specific embodiment, the pharmaceutical composition may also contain at least one known anticancer drug or pharmaceutically acceptable salts thereof, for the treatment of cancer.

The disclosure is also directed to methods for the preparation of novel compounds of Formula I (including Formulae II and III) or pharmaceutically acceptable salts, geometric isomers, enantiomers, diastereomers, racemates, isotope-substituted derivatives, solvates, hydrates or prodrugs thereof.

### DETAILED DESCRIPTION OF THE DISCLOSURE

It should be understood that the characteristics of the embodiments described herein can be arbitrarily combined to form the technical solution of this disclosure. The definition of each group herein can apply to any of the embodiments described herein. For example, the definitions of the substituents of alkyl herein apply to any of the embodiments described herein unless the substituents of alkyl are clearly defined in the embodiment.

The term “hydrogen (H)” as employed herein includes its isotopes D and T.

The term “alkyl” as used herein refers to alkyl itself or a straight or branched chain radical of up to ten carbons. Useful alkyl groups include straight-chain or branched C₁₋₁₀ alkyl groups, preferably C₁₋₆ alkyl groups. In some embodiments, alkyl is C₁₋₄ alkyl. Typical C₁₋₁₀ alkyl groups include methyl, ethyl, propyl, isopropyl, butyl, sec-butyl, tent-butyl, 3-pentyl, hexyl and octyl groups.

The term “alkoxy” as used herein refers to oxygen substituted by the above mentioned C₁₋₁₀ alkyl groups, preferred C₁₋₆ alkyl groups or C₁₋₄ alkyl groups, e.g., methoxy, ethoxy, etc. The alkyl in the alkoxy groups may be optionally substituted. Substituents of alkoxy groups include, without limitation, halogen, morpholino, amino (including alkylamino and dialkylamino), and carboxy (including esters thereof).

The term “amino” as used herein are —NR′R″, wherein R′ and R″ each are independently hydrogen, an optionally substituted C₁₋₁₀ alkyl, an optionally substituted cycloalkyl, an optionally substituted aryl or an optionally substituted heteroaryl; or R′ and R″ together with the N to which they are attached form an optionally substituted 4-7 membered cyclic amino group, which optionally comprises one or more (such as 2, 3) additional heteroatoms selected from a group consisting of O, N and S. Preferred amino includes NH₂, and amino group of which at least one of R′ and R″ is C₁₋₆ alkyl (preferably C₁₋₄ alkyl).

The term “oxo” as used herein refers to ═O.

The term “aryl” as used herein by itself or as part of another group refers to monocyclic, bicyclic or tricyclic aromatic groups containing 6 to 14 carbon atoms. Aryl may be substituted by one or more substituents as described herein.

Useful aryl groups include C₆₋₁₄ aryl groups, preferably C₆₋₁₀ aryl groups. Typical C₆₋₁₄ aryl groups include phenyl, naphthyl, phenanthryl, anthracyl, indenyl, azulyl, biphenyl, biphenylene and fluorenyl.

The term “carbocyclic group” as used herein is saturated and partially saturated cyclic carbocyclic groups consisted of carbon and hydrogen, include cycloalkyl and cycloalkenyl. Useful cycloalkyl groups are C₃₋₈ cycloalkyl. Useful partially saturated carbocyclic groups include C₃₋₈ cycloalkenyl. Typical cycloalkyl groups include cyclopropyl, cyclobutyl, cyclopentyl, cyclohexyl and cycloheptyl. Typical cycloalkenyl groups include cyclopentenyl, cyclohexenyl, cycloheptenyl and cyclooctenyl. carbocyclic group, such as C₃₋₈ cycloalkyl and C₃₋₈ cycloalkenyl, may be substituted by one or more substituents as described herein.

Useful halo or halogen groups include fluoro, chloro, bromo and iodo.

Useful acyl groups include C₁₋₆ acyl groups, such as acetyl. Acyl may be optionally substituted by group selected from halo, amino and aryl, wherein the amino and aryl may be optionally substituted. When acyl is substituted by halo, the number of halogen substituents may be in the range of 1-5. Examples of substituted acyls include chloroacetyl and pentafluorobenzoyl. When acyl is substituted by amino, amino group may be substituted by one or two substituents as described herein. In some embodiments, aminoacyl is —C(O)—NR′R″, wherein R′ and R″ each are independently hydrogen, an optionally substituted C₁₋₁₀ alkyl, an optionally substituted C₃₋₈ cycloalkyl, an optionally substituted aryl or an optionally substituted heteroaryl. Preferably, R′ and R″ each are independently hydrogen, an optionally substituted C₁₋₄ alkyl or an optionally substituted C₃₋₆ cycloalkyl. When the alkyl, cycloalkyl, aryl and heteroaryl groups in R′ and R″ are substituted, the substituents are as described in any embodiment herein, and the preferred substituents include halogen, hydroxyl, amino and alkyl etc.

Useful acylamino (amido) groups are any C₁₋₆ acyl (alkanoyl) attached to an amino nitrogen, e.g., acetamino, propionamido, butanoylamido, pentanoylamido and hexanoylamido, as well as aryl-substituted C₁₋₆ acylamino groups, e.g., benzoylamido. Useful acyl groups include C₁₋₆ acyl groups, such as acetyl. Acyl may be optionally substituted by group selected from aryl and halo, wherein the aryl may be optionally substituted. When acyl is substituted by halo, the number of halogen substituents may be in the range of 1-5. Examples of substituted acyls include chloroacetyl and pentafluorobenzoyl.

The term “heterocyclic group” as used herein refers to a saturated or partially saturated 3-7 membered monocyclic, or 7-10 membered bicyclic ring, spirocyclic ring or bridged ring system, which consists of carbon atoms and 1-4 heteroatoms independently selected from a group consisting of O, N, and S, wherein the nitrogen and/or sulfur heteroatoms can be optionally oxidized and the nitrogen can be optionally quaternized, and the term also includes any bicyclic ring system in which any of the above-defined heterocyclic rings is fused to a benzene ring. The heterocyclic group can be substituted on carbon atom or nitrogen atom if the resulting compound is stable. The heterocyclic group may be substituted by one or more substituents as described herein.

Useful saturated or partially saturated heterocyclic groups include tetrahydrofuryl, tetrahydropyranyl, piperidinyl, piperazinyl, 1,4-diazepanyl, pyrrolidinyl, imidazolidinyl, imidazolinyl, indolinyl, isodihydro Indolyl, quinuclidinyl, morpholinyl, oxazinyl, isochromanyl, chromanyl, pyrazolidinyl, pyrazolinyl, tetrahydroisoquinolinyl, tetronoyl, tetramoyl, dihydropyridine base, dihydropyrimidinyl, azetidinyl, oxetanyl, oxiranyl, which may be optionally substituted by one or more substituents as described herein.

The term “heteroaryl” as used herein refers to a group having 5 to 14 ring atoms, with 6, 10 or 14π electrons shared in a cyclic array. Ring atoms are carbon atoms and 1-3 heteroatoms selected from a group consisting of oxygen, nitrogen and sulfur. Heteroaryl may be optionally substituted by one or more substituents as described herein.

Useful heteroaryl groups include thienyl (thiophenyl), benzo[d]isothiazol-3-yl, benzo[b]thienyl, naphtho[2,3-b]thienyl, thianthrenyl, furyl (furanyl), pyranyl, isobenzofuranyl, chromenyl, xanthenyl, phenoxanthiinyl, pyrrolyl, imidazolyl, pyrazolyl, pyridyl (pyridinyl, including without limitation 2-pyridyl, 3-pyridyl, and 4-pyridyl), pyrazinyl, pyrimidinyl, pyridazinyl, indolyl, isoindolyl, 3H-indolyl, indolyl, indazolyl, purinyl, 4H-quinazinyl, isoquinolyl, quinoline Base, quinoxalinyl, phthalazinyl, naphthyridinyl, dihydronaphthyridinyl, quinazolinyl, cinnolinyl, pteridinyl, carbazolyl, β-carbolinyl, phenanthridinyl, acridine Base, naphthiazine (hetero)phenyl, phenanthroline, phenazinyl, isothiazolyl, phenothiazinyl, isoxazolyl, furazanyl, phenoxazinyl, 1,4-dihydroquinoxaline-2,3-dione, 7-aminoisocoumarin, pyridopyrimidin-4-one, tetrahydropyridopyrimidinyl, tetrahydropenta-membered [c]pyrazol-3-yl, benzo Isoxazolyl such as 1,2-benzisoxazol-3-yl, benzimidazolyl, 2-oxindolyl, thiadiazoyl, 2-oxobenzoimidazolyl, imidazopyridazinyl , imidazopyridyl, triazolopyridazinyl, triazolopyridyl, dihydropyridopyrimidinyl, tetrahydropyridopyrimidinyl, pyrazolopyridyl, pyrazolopyrimidinyl, pyrrolopyrimidine Base, pyrrolopyridyl, pyrrolopyrazinyl, pyridotriazinyl or triazolopyrazinyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

In this disclosure, unless otherwise described, when substituted, the alkyl, cycloalkyl, alkoxy, amido, carbonyl, heterocyclic group, aryl or heteroaryl as described in any embodiment herein may be substituted by one or more (such as 1, 2, 3 or 4) substituents selected from the group consisting of halogen, cyano, nitro, hydroxyl, carboxyl, C₁-C₆ amido, C₁-C₆ alkoxy, aryloxy, C₁-C₆ alkyl, C₁-C₆ acyl, C₆-C₁₀ aryl, C₃-C₈ cycloalkyl, heterocyclic group or heteroaryl and carbonyl, etc. The substituent itself may also be optionally substituted. Preferred substituents include without limitation halogen, carboxyl, C₁-C₆ amido, C₁-C₆ alkoxy, C₁-C₆ alkyl and C₁-C₆ acyl.

It should be understood that in each embodiment, when the substituent is a cycloalkyl, heterocyclic group, aryl or heteroaryl, the number thereof is usually 1. In addition, it should be understood that the connection or substitution between groups in the present invention should satisfy the bond valence theory; unless otherwise specified, when the bond valence theory is not satisfied, it is usually filled with H. The circles in each structural formula represent the number and position of double bonds satisfying the covalent bond theory.

Specifically, the disclosure provides compounds represented by Formula I:

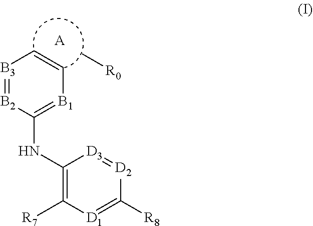

or pharmaceutically acceptable salts thereof, or geometric isomers, enantiomers, diastereomers, racemates, isotope-substituted derivatives, solvates, hydrates or prodrugs thereof, wherein:
A ring is an optionally substituted heterocyclic group or an optionally substituted heteroaryl;
R₀ is selected from an optionally substituted carbocyclic group and an optionally substituted heterocyclic group;
B₁ is selected from N and CR₁; B₂ is selected from N and CR₂; B₃ is selected from N and CR₃;
D₁ is selected from N and CR₄; D₂ is selected from N and CR₅; D₃ is selected from N and CR₆;
R₁, R₂, R₃, R₄, R₅, and R₆ are each independently selected from H, halogen, an optionally substituted alkyl, and an optionally substituted alkoxy;
R₇ is selected from halogen, an optionally substituted C₁-C₆ alkyl and an optionally substituted C₁-C₆ alkoxy;
R₈ is selected from an optionally substituted alkyl, an optionally substituted alkoxy, an optionally substituted amino, an optionally substituted heterocyclic group and an optionally substituted heteroaryl; or R₈ is connected to D₁ or to D₂ to form an optional substituted heterocyclic group or an optional substituted heteroaryl.

In some embodiments of the compound of Formula I, R₀ is connected at the ortho position of the ring carbon atom bridged by A ring and the ring containing B₁-B₃. In some embodiments, R₀ is connected to the nitrogen atom in A ring. In other embodiments, R₀ is connected to the carbon atom in A ring.

In some embodiments of the compound of Formula I, the compound of Formula I is represented by Formula Ia or Ib:

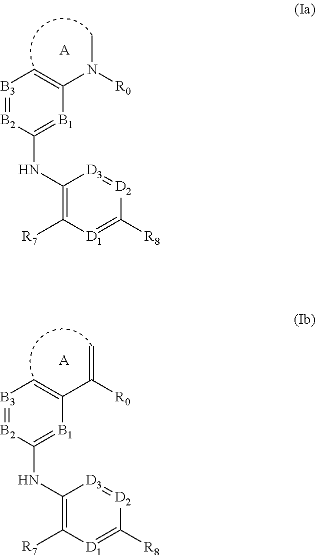

wherein, A ring, R₀, B₁, B₂, B₃, D₁, D₂, D₃, R₇ and R₈ are as defined in Formula I.

In some embodiments of the compound of Formulae I, Ia and Ib, A ring is an optionally substituted 6 membered heterocyclic group or an optionally substituted 6 membered heteroaryl.

In some embodiments of the compound of Formulae I, Ia and Ib, preferably, when A ring is substituted, the substituents can be 1, 2 or 3 groups selected from optionally substituted C₁-C₆ alkyl, optionally substituted C₁-C₆ alkoxy, halogen, cyano, oxo, optionally substituted C₃-C₇ cycloalkyl, optionally substituted heterocyclic group, optionally substituted C₁-C₆ acyl and —NRₐR_{b}, wherein Rₐ and R_{b} are each independently H or C₁-C₄ alkyl. When the said C₁-C₆ alkyl, C₁-C₆ alkoxy and C₁-C₆ acyl are substituted, the substituents can be 1-5 groups selected from halogen, hydroxyl and —NRₐR_{b}, wherein Rₐ and R_{b} are each independently H or C₁-C₄ alkyl. When the said C₃-C₇ cycloalkyl is substituted, the substituents can be 1, 2 or 3 groups selected from halogen, C₁-C₄ alkyl, halogenated C₁-C₄ alkyl, C₁-C₄ alkoxy, halogenated C₁-C₄ alkoxy, hydroxyl and —NRₐR_{b}, wherein Rₐ and R_{b} are each independently H or C₁-C₄ alkyl. The said heterocyclic group is 3-7 membered heterocyclic group, more preferably 3-7 membered nitrogen and/or oxygen-containing heterocyclic group, including but not limited to azetidinyl, oxetanyl, oxiranyl, tetrahydrofuranyl, tetrahydropyranyl, pyrrolidinyl, morpholinyl, piperazinyl and piperidinyl etc. When the said heterocyclic group is substituted, the substituents can be 1, 2 or 3 groups selected from halogen, C₁-C₄ alkyl, halogenated C₁-C₄ alkyl, C₁-C₄ alkoxy, halogenated C₁-C₄ alkoxy, hydroxyl and —NRₐR_{b}, wherein Rₐ and R_{b} are each independently H or C₁-C₄ alkyl. In some embodiments, the ring atom adjacent to the N substituted by R₀ is substituted by ═O.

In some embodiments of the compound of Formulae I, Ia and Ib, R₀ is optionally substituted cycloalkyl or optionally substituted cycloalkenyl, preferably, R₀ is optionally substituted C₃-C₇ cycloalkyl or optionally substituted C₃-C₇ cycloalkenyl. In some embodiments, R₀ is optionally substituted cyclopropyl, optionally substituted cyclobutyl, optionally substituted cyclopentyl, optionally substituted cyclohexyl, optionally substituted cycloheptyl, optionally substituted cyclopentenyl or optionally substituted cyclohexenyl. In some embodiments, the said cyclopentenyl group is cyclopent-1-en-1-yl. In some embodiments, R₀ is optionally substituted C₃-C₇ heterocyclic group. Preferred heterocyclic groups are 3-6 membered nitrogen and/or oxygen-containing heterocyclic groups, including azetidinyl, oxetanyl, oxirandyl, tetrahydrofuranyl, tetrahydropyranyl, pyrrolidinyl, morpholinyl, piperazinyl and piperidinyl, etc. When R₀ is substituted, the substituents can be 1, 2 or 3 groups selected from halogen, C₁-C₆ alkyl, halogenated C₁-C₆ alkyl, hydroxyl, C₁-C₆ alkoxy, halogenated C₁-C₆ alkoxy and —NRₐR_{b}, wherein Rₐ and R_{b} are each independently H or C₁-C₄ alkyl.

In some embodiments of the compound of Formulae I, Ia and Ib, B₁ is N or CR₁, B₂ is N, B₃ is N or CR₃; R₁ is preferably H, halogen or C₁-C₄ alkyl, R₃ is preferably H, halogen or C₁-C₄ alkyl. In some embodiments, B₁, B₂ and B₃ are each independently N or CH. Preferably, B₁ is N; B₂ is N; B₃ is CR₃, wherein R₃ is H or C₁-C₄ alkyl. More preferably, B₁ is N; B₂ is N; B₃ is CH.

In some embodiments of the compound of Formulae I, Ia and Ib, D₁, D₂ and D₃ are each independently N or CH; or D₁ is CR₄, D₂ is CR₅, and D₃ is CR₆. Preferably, R₄, R₅ and R₆ are each independently H, halogen and C₁-C₄ alkyl. Preferably, all of D₁, D₂ and D₃ are CH.

In some embodiments of the compound of Formulae I, Ia and Ib, the alkyl and alkoxy of R₁, R₂, R₃, R₄, R₅ and R₆ are each C₁-C₄ alkyl and C₁-C₄ alkoxy, when the said alkyl and alkoxy are substituted, the substituents can be 1, 2, 3, 4 or 5 groups independently selected from halogen, hydroxyl and —NRₐR_{b}, wherein Rₐ and R_{b} are each independently H or C₁-C₄ alkyl.

In some embodiments of the compound of Formulae I, Ia and Ib, R₇ is halogen, optionally substituted C₁-C₃ alkyl and optionally substituted C₁-C₃ alkoxy. Preferably, R₇ is halogen and optionally substituted methoxy; more preferably, R₇ is methoxy. Preferably, when the said C₁-C₃ alkyl and C₁-C₃ alkoxy are substituted, the substituents can be 1, 2, 3, 4 or 5 groups independently selected from halogen, hydroxyl and —NRₐR_{b}, wherein Rₐ and R_{b} are each independently H or C₁-C₄ alkyl. In some embodiments, R₇ is halogen, C₁-C₃ alkoxy (such as methoxy, ethoxy and propoxy) or halogenated C₁-C₃ alkoxy (such as trifluoromethoxy).

In some embodiments of the compound of Formulae I, Ia and Ib, R₈ is optionally substituted C₁-C₆ alkyl, optionally substituted C₁-C₆ alkoxy, optionally substituted C₁-C₆ alkylamino and optionally substituted heterocyclic group. The said heterocyclic group is 4-7 membered nitrogen and/or oxygen-containing heterocyclic group, including azetidinyl, oxetanyl, tetrahydrofuranyl, tetrahydropyranyl, pyrrolidinyl, morpholinyl, piperazinyl, 1,4-diazepanyl (such as 1,4-diazepan-1-yl) and piperidinyl, etc. Preferably, the substituents on R₈ can be 1-4 groups selected from C₁-C₆ alkyl, C₁-C₆ alkyl substituted with hydroxy, halogenated C₁-C₆ alkyl, C₁-C₆ alkoxy, C₁-C₆ acyl, heterocyclic group optionally substituted with 1-4 C₁-C₆ alkyl, halogen, —NRₐR_{b} and hydroxyl, wherein Rₐ and R_{b} are each independently H or C₁-C₆ alkyl. Preferably, the substituents on R₈ can be 1, 2 or 3 groups selected from hydroxyl, halogen, C₁-C₄ alkyl and halogenated C₁-C₄ alkyl. In preferred embodiments, R₈ is a 4-7 membered nitrogen-containing heterocyclic group optionally substituted by 1 or 2 substituents selected from —NRₐR_{b}, C₁-C₄ alkyl and halogenated C₁-C₄ alkyl, including azetidinyl, pyrrolidinyl, piperazinyl, 1,4-diazepanyl and piperidinyl.

In some embodiments of the compound of Formulae I, Ia and Ib, R₈ is connected with D₁ or D₂ to form an optionally substituted 4-7 membered heterocyclic group or an optionally substituted 5-14 membered heteroaryl. Preferred 4-7 membered heterocyclic groups include, but are not limited to, tetrahydrofuranyl, tetrahydropyranyl, pyrrolidinyl, morpholinyl, piperazinyl, 1,4-diazepanyl (such as 1,4-diazepan-1-yl) and piperidinyl, etc.; preferred 5-14 membered heteroaryl include, but are not limited to, furyl, pyranyl, pyrrolyl, imidazolyl, pyrazolyl, pyridyl, pyrazinyl, pyrimidinyl, pyridazinyl and indolizinyl, etc. When the said heterocyclic group and heteroaryl are substituted, the substituents can be 1, 2 or 3 groups selected from C₁-C₆ alkyl, C₁-C₆ acyl, heterocyclic group (4-7 membered heterocyclic group as described above) optionally substituted with 1-4 C₁-C₆ alkyl, halogen, —NRₐR_{b} and hydroxyl, wherein Rₐ and R_{b} are each independently H or C₁-C₆ alkyl.

In some embodiments of the compound of Formulae I, Ia and Ib, A ring is preferably an optionally substituted 6 membered nitrogen-containing heterocyclic group or an optionally substituted 6 membered nitrogen-containing heteroaryl, more preferably, the bicyclic ring formed by A ring and the fused aromatic ring containing B₁, B₂ and B₃ is selected from:

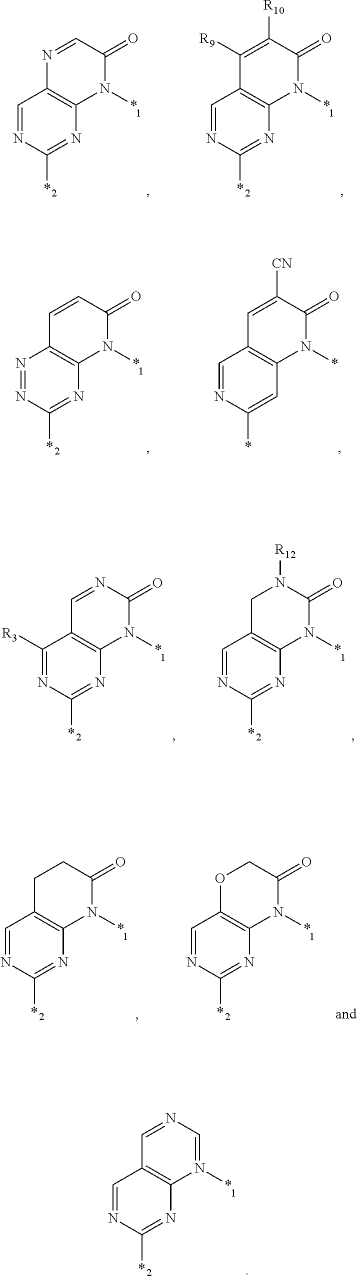

wherein, *₁ and *₂ refer to the attachment positions of the said groups to R₀ and —NH in the rest of the compound; R₃ is H or C₁-C₃ alkyl (preferably methyl); R₉ is H or C₁-C₃ alkyl (preferably methyl); R₁₀ is H, C₁-C₃ alkyl (preferably methyl), C₂-C₄ acyl (preferably acetyl) or cyano; R₁₂ is H or C₁-C₃ alkyl (preferably methyl).

Preferably, the compound of Formulae I, Ia and Ib are represented by Formula II (including Formulae II, IIb and IIc):

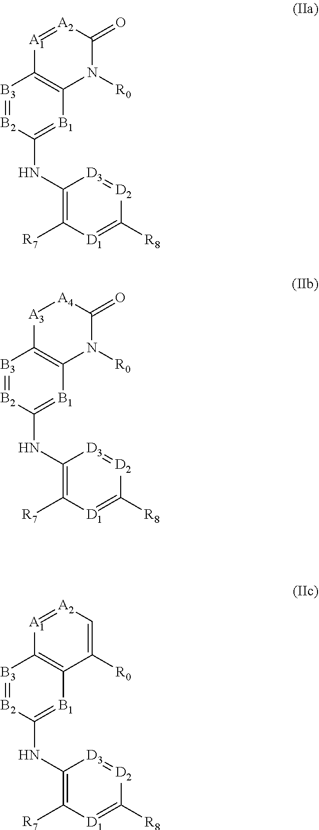

wherein, R₀, B₁, B₂, B₃, D₁, D₂, D₃, R₇ and R₈ are as defined in Formula I.
A₁ is selected from N and CR₉;
A₂ is selected from N and CR₁₀;
A₃ is selected from O, S, NR₁₁ and CR₁₃R′₁₃;
A₄ is selected from O, S, NR₁₂ and CR₁₄R′₁₄;
R₉ and R₁₀ are each independently H, halogen, cyano, optionally substituted C₁-C₆ alkyl, optionally substituted C₁-C₆ alkoxy or optionally substituted C₁-C₆ acyl;
R₁₁ and R₁₂ are each independently H or optionally substituted C₁-C₆ alkyl;
R₁₃, R′₁₃, R₁₄ and R′₁₄ are each independently H, optionally substituted C₁-C₆ alkyl or optionally substituted C₁-C₆ alkoxy.

In the compounds of Formulae IIa, IIb and IIc, R₀ is optionally substituted cycloalkyl or optionally substituted cycloalkenyl, preferably, R₀ is optionally substituted C₃-C₇ cycloalkyl or optionally substituted C₃-C₇ cycloalkenyl. In some embodiments, R₀ is optionally substituted cyclopropyl, optionally substituted cyclobutyl, optionally substituted cyclopentyl, optionally substituted cyclohexyl, optionally substituted cycloheptyl, optionally substituted cyclopentenyl or optionally substituted cyclohexenyl. In some embodiments, the said cyclopentenyl group is cyclopent-1-en-1-yl. In some embodiments, R₀ is optionally substituted C₃-C₇ heterocyclic group. Preferred heterocyclic groups are 3-6 membered nitrogen and/or oxygen-containing heterocyclic groups, including azetidinyl, oxetanyl, oxirandyl, tetrahydrofuranyl, tetrahydropyranyl, pyrrolidinyl, morpholinyl, piperazinyl and piperidinyl, etc. When R₀ is substituted, the substituents can be 1, 2 or 3 groups selected from halogen, C₁-C₆ alkyl, halogenated C₁-C₆ alkyl, hydroxyl, C₁-C₆ alkoxy, halogenated C₁-C₆ alkoxy and —NRₐR_{b}, wherein Rₐ and R_{b} are each independently H or C₁-C₄ alkyl.

In the compounds of Formulae IIa, IIb and IIc, preferably, B₁ is N or CR₁, B₂ is N, B₃ is N or CR₃; R₁ is preferably H, halogen or C₁-C₄ alkyl, R₃ is preferably H, halogen or C₁-C₄ alkyl. More preferably, B₁, B₂ and B₃ are each independently N or CH. Preferably, B₁ is N; B₂ is N; B₃ is CR₃, wherein R₃ is H or C₁-C₄ alkyl. More preferably, B₁ is N; B₂ is N; B₃ is CH.

In the compounds of Formulae IIa, IIb and IIc, preferably, D₁, D₂ and D₃ are each independently N or CH; or D₁ is CR₄, D₂ is CR₅, and D₃ is CR₆. Preferably, R₄, R₅ and R₆ are each independently H, halogen and C₁-C₄ alkyl. Preferably, all of D₁, D₂ and D₃ are CH.

In one of the foregoing embodiments of the compounds of Formulae IIa, IIb and IIc, R₇ is halogen, optionally substituted C₁-C₃ alkyl and optionally substituted C₁-C₃ alkoxy. Preferably, R₇ is halogen and optionally substituted methoxy. Preferably, when the said C₁-C₃ alkyl and C₁-C₃ alkoxy are substituted, the substituents can be 1, 2, 3, 4 or 5 groups independently selected from halogen, hydroxyl and —NRₐR_{b}, wherein Rₐ and R_{b} are each independently H or C₁-C4 alkyl. In some embodiments, R₇ is halogen, C₁-C₃ alkoxy (such as methoxy, ethoxy and propoxy) or halogenated C₁-C₃ alkoxy (such as trifluoromethoxy).

In one of the foregoing embodiments of the compounds of Formulae IIa, IIb and IIC, R₈ is optionally substituted C₁-C₆ alkyl, optionally substituted C₁-C₆ alkoxy, optionally substituted C₁-C₆ alkylamino and optionally substituted heterocyclic group. The said heterocyclic is 4-7 membered nitrogen and/or oxygen-containing heterocyclic group, preferably 4-6 membered nitrogen and/or oxygen-containing heterocyclic group, including azetidinyl, oxetanyl, tetrahydrofuranyl, tetrahydropyranyl, pyrrolidinyl, morpholinyl, piperazinyl, 1,4-diazepanyl (such as 1,4-diazepan-1-yl) and piperidinyl, etc. Preferably, the substituents on R₈ can be 1-4 groups selected from C₁-C₆ alkyl, C₁-C₆ alkyl substituted with hydroxy, halogenated C₁-C₆ alkyl, C₁-C₆ alkoxy, C₁-C₆ acyl, heterocyclic group (4-6 membered nitrogen and/or oxygen-containing heterocyclic group as described above) optionally substituted with 1-4 C₁-C₆ alkyl, halogen, —NR a R_{b} and hydroxyl, wherein Rₐ and R_{b} are each independently H or C₁-C₆ alkyl. Preferably, the substituents on R₈ can be 1, 2 or 3 groups selected from hydroxyl, halogen, C₁-C₄ alkyl and halogenated C₁-C₄ alkyl. In preferred embodiments, R₈ is a 4-7 membered nitrogen-containing heterocyclic group optionally substituted by 1 or 2 substituents selected from —NRₐR_{b}, C₁-C₄ alkyl and halogenated C₁-C₄ alkyl, including azetidinyl, pyrrolidinyl, piperazinyl, 1,4-diazepanyl and piperidinyl.

In one of the foregoing embodiments of the compounds of formula I, R₈ is connected with D₁ or D₂ to form an optionally substituted 4-7 membered heterocyclic group or an optionally substituted 5-14 membered heteroaryl. Preferred 4-7 membered heterocyclic groups include, but are not limited to, tetrahydrofuranyl, tetrahydropyranyl, pyrrolidinyl, morpholinyl, piperazinyl, 1,4-diazepanyl (such as 1,4-diazepan-1-yl) and piperidinyl, etc.; preferred 5-14 membered heteroaryl include, but are not limited to, furyl, pyranyl, pyrrolyl, imidazolyl, pyrazolyl, pyridyl, pyrazinyl, pyrimidinyl, pyridazinyl and indolizinyl, etc. When the said heterocyclic group and heteroaryl are substituted, the substituents can be 1, 2 or 3 groups selected from C₁-C₆ alkyl, C₁-C₆ acyl, heterocyclic group (4-7 membered heterocyclic group as described above) optionally substituted with 1-4 C₁-C₆ alkyl, halogen, —NR a R_{b} and hydroxyl, wherein R a and R_{b} are each independently H or C₁-C₆ alkyl.

In one of the foregoing embodiments of the compounds of Formula IIa, when the respective C₁-C₆ alkyl, C₁-C₆ alkoxy and C₁-C₆ acyl of R₉ and R₁₀ are substituted, the substituents can be 1, 2, 3, 4 or 5 groups selected from halogen, hydroxyl and —NRₐR_{b}, wherein Rₐ and R_{b} are each independently H or C₁-C₄ alkyl. Preferred R₉ and R₁₀ are each independently H, C₁-C₄ alkyl, CN or C₂-C₄ acyl. In some embodiments, R₉ is H or C₁-C₃ alkyl (preferably methyl); R₁₀ is H, C₁-C₃ alkyl (preferably methyl), cyano or C₂-C₄ acyl (preferably acetyl).

In one of the foregoing embodiments of the compounds of Formula IIa, preferably, A₁ and A₂ are each independently N, CH, C—CN, C-(C₁-C₃ alkyl) or C-(C₂-C₄ acyl). In some embodiments, A₁ is N and A₂ is CR₁₀, wherein R₁₀ is selected from H and C₁-C₃ alkyl. In some embodiments, A₁ is CR₉, A₂ is N, wherein R₉ is selected from H and C₁-C₃ alkyl. In some embodiments, A₁ is CR₉, A₂ is CR₁₀, wherein, R₉ is selected from H and C₁-C₃ alkyl, R₁₀ is selected from H, C₁-C₃ alkyl, cyano and C₂-C₄ acyl.

In one of the foregoing embodiments of the compounds of Formula IIb, when the respective C₁-C₆ alkyl group of Ru and R₁₂ are substituted, the substituents can be 1, 2, 3, 4 or 5 groups selected from halogen, hydroxyl and —NRₐR_{b}, wherein Rₐ and R_{b} are each independently H or C₁-C₄ alkyl.

In one of the foregoing embodiments of the compounds of Formula IIb, When the respective C₁-C₆ alkyl and C₁-C₆ alkoxy of R₁₃, R′₁₃, R₁₄ and R′₁₄ are substituted, the substituents can be 1, 2, 3, 4 or 5 groups selected from halogen, hydroxyl and —NRₐR_{b}, wherein Rₐ and R_{b} are each independently H or C₁-C₄ alkyl.

In one of the foregoing embodiments of the compounds of Formula IIb, A₃ is selected from O, NR₁₁ and CR₁₃R′₁₃, A₄ is selected from O, NR₁₂ and CR₁₄R′₁₄; preferably, A₃ is selected from O and CR₁₃R′₁₃, and A₄ is selected from O, NR₁₂ and CR₁₄R′₁₄. Preferably, R₁₁, R₁₂, R₁₃, R′₁₃, R₁₄ and R′₁₄ are each independently H or C₁-C₃ alkyl; preferably both R₁₁ and R₁₂ are H, at least one of R₁₃ and R′₁₃ is H, and at least one of R₁₄ and R′₁₄ is H. In some embodiments, A₃ and A₄ are each independently O, CH₂ or NH.

In one of the foregoing embodiments of the compounds of Formula IIc, A₁ and A₂ are each independently N, CH, C—CN, C-(C₁-C₃ alkyl) or C-(C₂-C₄ acyl). Preferably, A₁ and A₂ are each independently N or CH. More preferably, A₁ is CH, A₂ is CN.

In some embodiments of the compound of Formula IIa, preferably, the fused bicyclic ring containing A₁, A₂, B₁, B₂ and B₃ are selected from:

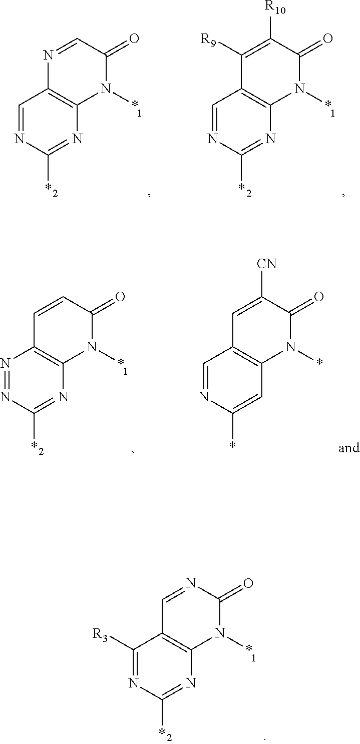

wherein, *₁ and *₂ refer to an attachment position of the said groups to R₀ and —NH in the rest of the compound; R₃ is H or C₁-C₃ alkyl (preferably methyl); R₉ is H or C₁-C₃ alkyl (preferably methyl); R₁₀ is H, C₁-C₃ alkyl (preferably methyl), cyano or C₂-C₄ acyl (preferably acetyl).

In some embodiments of the compound of Formula IIb, preferably, the fused bicyclic ring containing A₃, A₄, B₁, B₂ and B₃ are selected from:

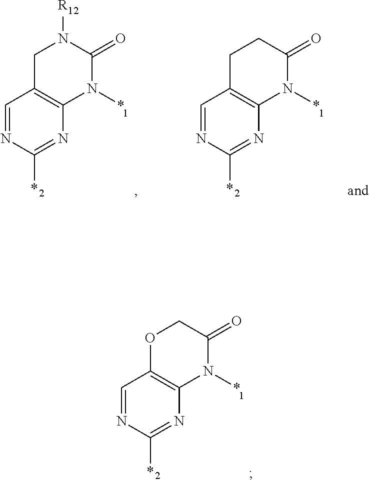

wherein, *₁ and *₂ refer to an attachment position of the said groups to R₀ and —NH in the rest of the compound; R₁₂ is H or C₁-C₃ alkyl (preferably methyl).

In some embodiments of the compound of Formula IIc, preferably, the fused bicyclic ring containing A₁, A₂, B₁, B₂ and B₃ are selected from:

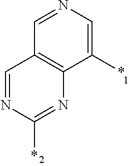 [SMILES: *c1ncc2cncc(*)c2n1]

In one or more of the foregoing embodiments, the compound of Formula II is represented by Formula III (including Formulae IIIa, IIIb, IIIc, IIId and IIIe):

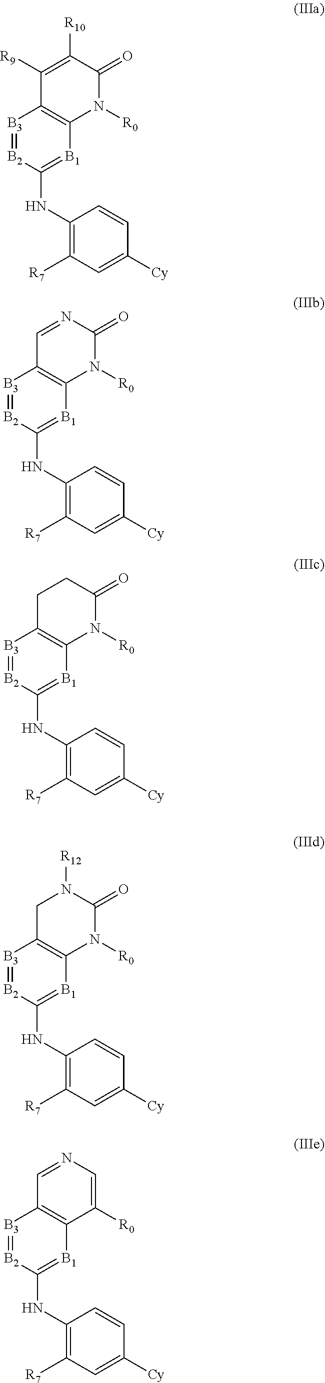

wherein, R₀, B₁, B₂, B₃, R₇, R₉, R₁₀ and Ru are as defined in Formula I, Ia, Ib, IIa, IIb or IIc.
Cy is selected from optionally substituted heterocyclic group.

In the compounds of Formulae IIIa, IIIb, IIIc, IIId and IIIe, R₀ is optionally substituted cycloalkyl or optionally substituted cycloalkenyl, preferably, R₀ is optionally substituted C₃-C₇ cycloalkyl or optionally substituted C₃-C₇ cycloalkenyl. In some embodiments, R₀ is optionally substituted cyclopropyl, optionally substituted cyclobutyl, optionally substituted cyclopentyl, optionally substituted cyclohexyl, optionally substituted cycloheptyl, optionally substituted cyclopentenyl or optionally substituted cyclohexenyl. In some embodiments, the said cyclopentenyl group is cyclopent-1-en-1-yl . In some embodiments, R₀ is optionally substituted C₃-C₇ heterocyclic group. Preferred heterocyclic groups are 3-6 membered nitrogen and/or oxygen-containing heterocyclic groups, including azetidinyl, oxetanyl, oxirandyl, tetrahydrofuranyl, tetrahydropyranyl, pyrrolidinyl, morpholinyl, piperazinyl and piperidinyl, etc. When R₀ is substituted, the substituents can be 1, 2 or 3 groups selected from halogen, C₁-C₆ alkyl, halogenated C₁-C₆ alkyl, hydroxyl, C₁-C₆ alkoxy, halogenated C₁-C₆ alkoxy and —NRₐR_{b}, wherein Rₐ and R_{b} are each independently H or C₁-C₄ alkyl.

In the compounds of Formulae IIIa, IIIb, IIIc, IIId and IIIe, R₇ is halogen, optionally substituted C₁-C₃ alkyl and optionally substituted C₁-C₃ alkoxy. Preferably, R₇ is halogen and optionally substituted methoxy. Preferably, when the said C₁-C₃ alkyl and C₁-C₃ alkoxy are substituted, the substituents can be 1, 2, 3, 4 or 5 groups independently selected from halogen, hydroxyl and —NRₐR_{b}, wherein Rₐ and R_{b} are each independently H or C₁-C₄ alkyl. In some embodiments, R₇ is halogen, C₁-C₃ alkoxy (such as methoxy, ethoxy and propoxy) or halogenated C₁-C₃ alkoxy (such as trifluoromethoxy).

In one or more of the foregoing embodiments of the compound of Formula IIIa, when the respective C₁-C₆ alkyl, C₁-C₆ alkoxy and C₁-C₆ acyl of R₉ and R₁₀ are substituted, the substituents can be 1, 2, 3, 4 or 5 groups selected from halogen, hydroxyl and —NRₐR_{b}, wherein Rₐ and R_{b} are each independently H or C₁-C₄ alkyl. Preferred R₉ and R₁₀ are each independently H, C₁-C₄ alkyl, CN or C₂-C₄ acyl. In some embodiments, R₉ is H or C₁-C₃ alkyl (preferably methyl); R₁₀ is H, C₁-C₃ alkyl (preferably methyl), cyano or C₂-C₄ acyl (preferably acetyl).

In one or more of the foregoing embodiments of the compound of Formula IIId, when the C₁-C₆ alkyl group of R₁₂ is substituted, the substituents can be 1, 2, 3, 4 or 5 groups selected from halogen, hydroxyl and —NRₐR_{b}, wherein Rₐ and R_{b} are each independently H or C₁-C₄ alkyl.

In the compounds of Formulae IIIa, IIIb, IIIc and IIId, preferably, B₁ is N or CR₁, B₂ is N, B₃ is N or CR₃; R₁ is preferably H, halogen or C₁-C₄ alkyl, R₃ is preferably H, halogen or C₁-C₄ alkyl. More preferably, B₁, B₂ and B₃ are each independently N or CH. Preferably, B₁ is N; B₂ is N; B₃ is CR₃, wherein R₃ is H or C₁-C₄ alkyl. More preferably, B₁ is N; B₂ is N; B₃ is CH.

In one or more of the foregoing embodiments of the compound of Formulae IIIa, IIIb, IIIc and IIId, Cy is an optionally substituted 4-7 membered heterocyclic group, such as optionally substituted 4-7 membered nitrogen and/or oxygen-containing heterocyclic group, including azetidinyl, oxetanyl, tetrahydrofuranyl, tetrahydropyranyl, pyrrolidinyl, morpholinyl, piperazinyl, 1,4-diazepan-1-yl and piperidinyl, etc. Preferably, Cy is optionally substituted piperazinyl, optionally substituted piperidinyl, optionally substituted morpholinyl or optionally substituted 1,4-diazepan-1-yl. Preferably, when Cy is substituted, the substituents can be 1, 2 or 3 groups selected from C₁-C₆ alkyl, C₁-C₆ alkyl substituted with hydroxy, halogenated C₁-C₆ alkyl, C₁-C₆ alkoxy, C₁-C₆ acyl, heterocyclic group (4-6 membered nitrogen and/or oxygen-containing heterocyclic group as described above) optionally substituted with 1-4 C₁-C₆ alkyl, halogen, —NRₐR_{b} and hydroxyl, wherein Rₐ and R_{b} are each independently H or C₁-C₆ alkyl. More preferably, Cy is piperazinyl optionally substituted by 1-3 substituents selected from C₁-C₆ alkyl and C₁-C₆ alkyl substituted with hydroxyl, piperidinyl optionally substituted by 1 substituent selected from C₁-C₆ alkyl and —NRₐR_{b}, 1,4-diazepan-1-yl optionally substituted by 1-3 C₁-C₆ alkyl and morpholinyl optionally substituted by 1-3 C₁-C₆ alkyl; wherein Rₐ and R_{b} are each independently H or C₁-C₄ alkyl.

The preferred compounds of Formula I include, without limitation:
8-cyclopentyl-2-((2-methoxy-4-(4-methylpiperazin-1-yl)phenyl)amino)pteridin-7(8H)-one (Example 1);
1-cyclopentyl-7-((2-methoxy-4-(4-methylpiperazin-1-yl)phenyl)amino)-3,4-dihydropyrimido[4,5-d]pyrimidin-2(1H)-one (Example 2);
1-cyclopropyl-7-(2-methoxy-4-(4-methylpiperazin-1-yl)phenyl)amino)-3,4-dihydropyrimido[4,5-d]pyrimidin-2(1H)-one (Example 3);
1-cyclopentyl-7-((2-methoxy-4-(4-methylpiperazin-1-yl)phenyl)amino)-3-methyl-3,4-dihydropyrimido[4,5-d]pyrimidin-2(1H)-one (Example 4);
1-cyclopentyl-7-((2-methoxy-4-(4-methylpiperazin-1-yl)phenyl)amino)pyrimido[4,5-d]pyrimidin-2(1H)-one (Example 5);
1-cyclohexyl-7-((2-methoxy-4-(4-methylpiperazin-1-yl)phenyl)amino)pyrimido[4,5-d]pyrimidin-2(1H)-one (Example 6);
1-cyclopentyl-7-((2-ethoxy-4-(4-methylpiperazin-1-yl)phenyl)amino)pyrimido[4,5-d]pyrimidin-2(1H)-one (Example 7);
1-cyclopentyl-7-((2-isopropoxy-4-(4-methylpiperazin-1-yl)phenyl)amino)pyrimido[4,5-d]pyrimidin-2(1H)-one (Example 8);
1-cyclopentyl-7-((4-(4-methylpiperazin-1-yl)-2-(trifluoromethoxy)phenyl)amino)pyrimido[4,5-d]pyrimidin-2(1H)-one (Example 9);
1-cyclopentyl-7-((2-methyl-4-(4-methylpiperazin-1-yl)phenyl)amino)pyrimido[4,5-d]pyrimidin-2(1H)-one (Example 10);
7-((2-chloro-4-(4-methylpiperazin-1-yl)phenyl)amino)-1-cyclopentylpyrimido[4,5-d]pyrimidin-2(1H)-one (Example 11);
7-((2-bromo-4-(4-methylpiperazin-1-yl)phenyl)amino)-1-cyclopentylpyrimido[4,5-d]pyrimidin-2(1H)-one (Example 12);
1-cyclopentyl-7-((2-methoxy-4-(4-methylpiperazin-1-yl)phenyl)amino)-5-methylpyrimido[4,5-d]pyrimidin-2(1H)-one (Example 13);
1-cyclobutyl-7-((2-methoxy-4-(4-methylpiperazin-1-yl)phenyl)amino)pyrimido[4,5-d]pyrimidin-2(1H)-one (Example 14);
1-cyclopropyl-7-((2-methoxy-4-(4-methylpiperazin-1-yl)phenyl)amino)pyrimido[4,5-d]pyrimidin-2(1H)-one (Example 15);
7-((2-methoxy-4-(4-methylpiperazin-1-yl)phenyl)amino)-1-(tetrahydro-2H-pyran-4-yl)pyrimido[4,5-d]pyrimidin-2(1H)-one (Example 16);
7-((2-methoxy-4-(4-methylpiperazin-1-yl)phenyl)amino)-1-(piperidin-4-yl)pyrimido[4,5-d]pyrimidin-2(1H)-one (Example 17);
7-((2-methoxy-4-(4-methylpiperazin-1-yl)phenyl)amino)-1-(1-methylpiperidin-4-yl)pyrimido[4,5-d]pyrimidin-2(1H)-one (Example 18);
8-cyclopentyl-2-((2-methoxy-4-(4-methylpiperazin-1-yl)phenyl)amino)pyrido[2,3-d]pyrimidin-7(8H)-one (Example 19);
8-cyclopentyl-2-((2-methoxy-4-(4-methylpiperazin-1-yl)phenyl)amino)-6-methylpyrido[2,3-d]pyrimidin-7(8H)-one (Example 20);
8-cyclopropyl-2-(2-methoxy-4-(4-methylpiperazin-1-yl)phenyl)amino)-6-methylpyrido[2,3-d]pyrimidin-7(8H)-one (Example 21);
8-cyclohexyl-2-((2-methoxy-4-(4-methylpiperazin-1-yl)phenyl)amino)pyrido[2,3-d]pyrimidin-7(8H)-one (Example 22);
8-cyclopropyl-2-((2-methoxy-4-(4-methylpiperazin-1-yl)phenyl)amino)pyrido[2,3-d]pyrimidin-7(8H)-one (Example 23);
5-cyclopentyl-3-((2-methoxy-4-(4-methylpiperazin-1-yl)phenyl)amino)pyrido[2,3-e][1,2,4]triazin-6(5H)-one (Example 24);
1-cyclopentyl-7-((2-methoxy-4-(4-methylpiperazin-1-yl)phenyl)amino)-2-oxo-1,2-dihydro-1,6-naphthyridine-3-carbonitrile (Example 25);
8-cyclopentyl-2-((2-methoxy-4-(4-methylpiperazin-1-yl)phenyl)amino)-7-oxo-7,8-dihydropyrido[2,3-d]pyrimidine-6-carbonitrile (Example 26);
8-cyclopentyl-2-((2-methoxy-4-(4-methylpiperazin-1-yl)phenyl)amino)-5-methylpyrido[2,3-d]pyrimidin-7(8H)-one (Example 27);
8-cyclopentyl-2-((2-methoxy-4-(4-methylpiperazin-1-yl)phenyl)amino)-5,8-dihydropyrido[2,3-d]pyrimidin-7(6H)-one (Example 28);
8-cyclohexyl-2-((2-methoxy-4-(4-methylpiperazin-1-yl)phenyl)amino)-5,8-dihydropyrido[2,3-d]pyrimidin-7(6H)-one (Example 29);
8-cyclopropyl-2-(2-methoxy-4-(4-methylpiperazin-1-yl)phenyl)amino)-5,8-dihydropyrido[2,3-d]pyrimidin-7(6H)-one (Example 30);
8-cyclopentyl-2-((2-methoxy-4-(4-methylpiperazin-1-yl)phenyl)amino)-6H-pyrimido[5,4-b][1,4]oxazin-7(8H)-one (Example 31);
7-((2-bromo-4-(4-methylpiperazin-1-yl)phenyl)amino)-1-cyclopentyl-3,4-dihydropyrimido[4,5-d]pyrimidin-2(1H)-one (Example 32);
7-((2-methoxy-4-(4-methylpiperazin-1-yl)phenyl)amino)-1-(tetrahydro-2H-pyran-4-yl)-3,4-dihydropyrimido[4,5-d]pyrimidin-2(1H)-one (Example 33);
8-cyclopentyl-N-(2-methoxy-4-(4-methylpiperazin-1-yl)phenyl)pyrido[4,3-d]pyrimidin-2-amine (Example 34);
8-(cyclopent-1-en-1-yl)-N-(2-methoxy-4-(4-methylpiperazin-1-yl)phenyl)pyrido[4,3-d]pyrimidin-2-amine (Example 35);
6-acetyl-8-cyclopentyl-2-((2-methoxy-4-(4-methylpiperazin-1-yl)phenyl)amino)-5-methylpyrido[2,3-d]pyrimidin-7(8H)-one (Example 36);
6-acetyl-8-cyclopentyl-2-((2-methoxy-4-(piperazin-1-yl)phenyl)amino)-5-methylpyrido[2,3-d]pyrimidin-7(8H)-one (Example 37);
8-cyclohexyl-2-((2-methoxy-4-(4-methylpiperazin-1-yl)phenyl)amino)-6-methylpyrido[2,3-d]pyrimidin-7(8H)-one (Example 38);
8-cyclobutyl-2-((2-methoxy-4-(4-methylpiperazin-1-yl)phenyl)amino)-6-methylpyrido[2,3-d]pyrimidin-7(8H)-one (Example 39);
8-cycloheptyl-2-((2-methoxy-4-(4-methylpiperazin-1-yl)phenyl)amino)-6-methylpyrido[2,3-d]pyrimidin-7(8H)-one (Example 40);
8-cycloheptyl-2-((2-methoxy-4-(4-methylpiperazin-1-yl)phenyl)amino)pyrido[2,3-d]pyrimidin-7(8H)-one (Example 41);
or pharmaceutically acceptable salts thereof, or geometric isomers, enantiomers, diastereomers, racemates, isotope-substituted derivatives, solvates, hydrates or prodrugs thereof

Some of the compounds of the present disclosure may exist as stereoisomers including optical isomers. The disclosure includes all stereoisomers and the racemic mixtures of such stereoisomers as well as the individual enantiomers that may be separated according to methods that are well known to those of ordinary skill in the art.

Examples of pharmaceutically acceptable salts include inorganic and organic acid salts, such as hydrochloride, hydrobromide, phosphate, sulphate, citrate, lactate, tartrate, maleate, fumarate, mandelate and oxalate; and inorganic and organic base salts formed with bases, such as sodium hydroxy, tris(hydroxymethyl)aminomethane (TRIS, tromethamine) and N-methyl-glucamine.

Examples of prodrugs of the compounds of the disclosure include the simple esters of carboxylic acid-containing compounds (e.g., those obtained by condensation with a C₁-C₄ alcohol according to methods known in the art); esters of hydroxy containing compounds (e.g., those obtained by condensation with a C₁-C₄ carboxylic acid, C₃-C₆ diacid or anhydride thereof, such as succinic anhydride and fumaric anhydride according to methods known in the art); imines of amino containing compounds (e.g., those obtained by condensation with a C₁-C₄ aldehyde or ketone according to methods known in the art); carbamate of amino containing compounds, such as those described by Leu, et al., (J. Med. Chem. 42:3623-3628 (1999)) and Greenwald, et al., (J. Med. Chem. 42:3657-3667 (1999)); and acetals and ketals of alcohol-containing compounds (e.g., those obtained by condensation with chloromethyl methyl ether or chloromethyl ethyl ether according to methods known in the art).

The present disclosure also includes all appropriate isotopic variations of the compounds or pharmaceutically acceptable salts thereof. An isotopic change in a compound or a pharmaceutically acceptable salt thereof of the present disclosure indicates that at least one atom is replaced by an atom having the same atomic number but an atomic mass different from that normally found in nature. Isotopes that may be labeled into the compounds or pharmaceutically acceptable salts thereof include, but are not limited to, isotopes of H, C, N and O, such as ²H, ³H, ¹¹C, ¹³C, ¹⁴C, ¹⁵N, ¹⁷O, ¹⁸O, ³⁵S, ¹⁸F, ³⁶Cl, ¹²³I and ¹²⁵I. Appropriate isotopic derivatives of compounds or pharmaceutically acceptable salts thereof of the present disclosure may be prepared by conventional techniques using appropriate isotopic derivatives of appropriate reagents.

The compounds of this disclosure may be prepared using methods known to those skilled in the art, or the novel methods of this disclosure. Specifically, the compounds of this disclosure with Formula I can be prepared as illustrated by the exemplary reaction in Scheme 1. Reaction of ethyl 4-chloro-2-(methylthio)pyrimidine-5-carboxylate and cyclopentanamine under the catalysis of triethylamine in dichloromethane at room temperature produced ethyl 4-(cyclopentylamino)-2-(methylthio)pyrimidine-5-carboxylate. Reduction of ethyl 4-(cyclopentylamino)-2-(methylthio)pyrimidine-5-carboxylate with lithium aluminum hydride produced (4-(cyclopentylamino)-2-(methylthio)pyrimidin-5-yl)methanol. Reflux reaction of (4-(cyclopentylamino)-2-(methylthio)pyrimidin-5-yl)methanol in thionyl chloride produced 5-(chloromethyl)-N-cyclopentyl-2-(methylthio)pyrimidin-4-amine. Reaction of 5-(chloromethyl)-N-cyclopentyl-2-(methylthio)pyrimidin-4-amine and NH₃ in tetrahydrofuran at room temperature produced 5-(aminomethyl)-N-cyclopentyl-2-(methylthio)pyrimidin-4-amine. Reaction of 5-(aminomethyl)-N-cyclopentyl-2-(methylthio)pyrimidin-4-amine and 1,1′-carbonyldiimidazole produced 1-cyclopentyl-7-(methylthio)-3,4-dihydropyrimido[4,5-d]pyrimidin-2(1H)-one. Oxidation of 1-cyclopentyl-7-(methylthio)-3,4-dihydropyrimido[4,5-d]pyrimidin-2(1H)-one with 3-chloroperoxyb enzoic acid produced 1-cyclopentyl -7-(methylsulfinyl)-3,4-dihydropyrimido[4,5-d]pyrimidin-2(1H)-one. Reaction of 1-cyclopentyl-7-(methyl sulfinyl)-3,4-dihydropyrimido[4,5-d]pyrimidin-2(1H)-one and 2-methoxy-4-(4-methylpiperazin-1-yl)aniline under the catalysis of trifluoroacetic acid produced 1-cyclopentyl-7-((2-methoxy-4-(4-methylpiperazin-1-yl)phenyl)amino)-3,4-dihydropyrimido[4,5-d]pyrimidin-2(1H)-one. Oxidation of 1-cyclopentyl-7-((2-methoxy-4-(4-methylpiperazin-1-yl)phenyl)amino)-3,4-dihydropyrimido[4,5-d]pyrimidin-2(1H)-one with potassium tert-butoxide produced 8-cyclopentylethoxy-4-(4-methylpiperazin-1-yl)ph enyl)amino)pteridin-7(8H)-one.

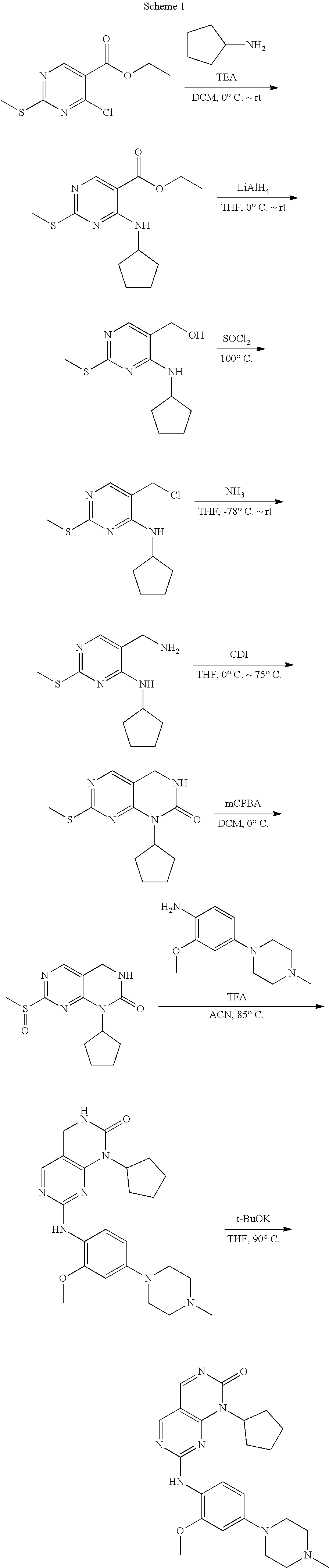

Other related compounds can be prepared using similar methods. For example, replacement of cyclopentanamine with cyclohexanamine produced the targeted compound 1-cyclohexyl-7-((2-methoxy-4-(4-methylpiperazin-1-yl)phenyl)amino)pyrimido[4,5-d]pyrimidin-2(1H)-one. Replacement of 2-methoxy-4-(4-methylpiperazin-1-yl)aniline with 2-ethoxy-4-(4-methylpiperazin- 1-yl)aniline produced the targeted compound 1-cyclopentyl-7-((2-ethoxy-4-(4-methylpiperazin-1-yl)phenyl)amino)pyrimido[4,5-d]pyrimidin-2(1H)-one. Replacement of 2-methoxy-4-(4-methylpiperazin-1-yl)aniline with 2-isopropoxy-4-(4-methylpiperazin-1-yl)aniline produced the targeted compound 1-cyclopentyl-7-((2-isopropoxy-4-(4-methylpiperazin-1-yl)phenyl)amino)pyrimido[4,5-d]pyrimidin-2(1H)-one. Replacement of 2-methoxy-4-(4-methylpiperazin-1-yl)aniline with 2-methyl-4-(4-methylpiperazin-1-yl)aniline produced the targeted compound 1-cyclopentyl-7-((2-methyl-4-(4-methylpiperazin-1-yl)phenyl)amino)pyrimido[4,5-d]pyrimidin-2(1H)-one. Replacement of ethyl 4-(cyclopentylamino)-2-(methylthio)pyrimidine-5-carboxylate with ethyl 4-(cyclopentylamino)-6-methyl-2-(methylthio)pyrimidine-5-carboxylate produced the targeted compound 1-cyclopentyl-742-methoxy-4-(4-methylpiperazin-1-yl)phenyl)amino)-5-methylpyrimido[4,5-d]pyrimidin-2(1H)-one. Replacement of cyclopentanamine with cyclobutanamine produced the targeted compound 1-cyclobutyl-7-((2-methoxy-4-(4-methylpiperazin-1-yl)phenyl)amino)pyrimido[4,5-d]pyrimidin-2(1H)-one.

The compounds of this disclosure can be prepared as illustrated by the exemplary reaction in Scheme 2. Oxidation of (4-(cyclopentylamino)-2-(methylthio)pyrimidin-5-yl)methanol with manganese dioxide produced 4-(cyclopentylamino)-2-(methylthio)pyrimidine-5-carb aldehyde. Wittig reaction of 4-(cyclopentylamino)-2-(methylthio)pyrimidine-5-carbaldehyde and ethyl 2-(triphenyl-15-phosphinidyl) acetate produced ethyl 3-(4-(cyclopentylamino)-2-(methylthio)pyrimidin-5-yl)acrylate. Reaction of ethyl 3-(4-(cyclopentylamino)-2-(methylthio)pyrimidin-5-yl)acrylate and 1,8-diazabicycloundec-7-ene produced 1-cyclopentyl-7-(methylthio)-3,4-dihydropyrimidino [4,5-d]pyrimidin-2(1H)-one. Oxidation of 1-cyclopentyl-7-(methylthio)-3,4-dihydropyrimidino [4,5-d]pyrimidin-2(1H)-one with 3-chloroperoxybenzoic acid produced 8-cyclopentyl-2-(methylsulfinyl)pyrido[2,3-d]pyrimidin-7(8H)-one. Reaction of 8-cyclopentyl-2-(methylsulfinyl)pyrido[2,3-d]pyrimidin-7(8H)-one and 2-methoxy-4-(4-methylpiperazin-1-yl)aniline under the catalysis of trifluoroacetic acid produced 8-cyclopentyl-2-((2-methoxy-4-(4-methylpiperazin-1-yl)phenyl)amino)pyrido[2,3-d]pyrimidin-7(8H)-one.

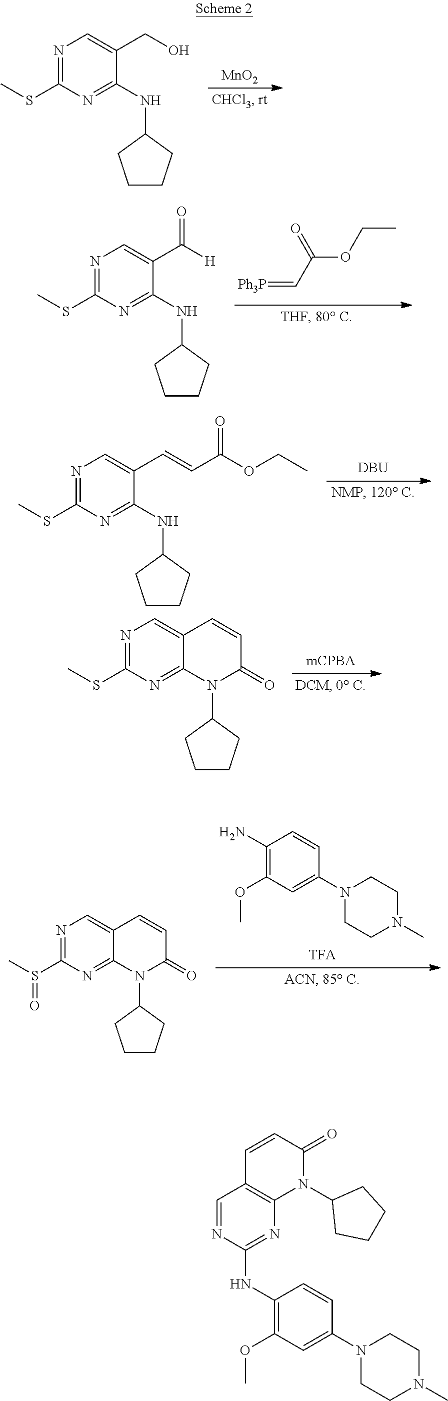

Other related compounds can be prepared using similar methods. For example, replacement of ethyl (triphenylphosphoranylidene)acetate with 2-phosphonopropionic acid triethyl ester produced the targeted compound 8-cyclopentyl-2-((2-methoxy-4-(4-methylpiperazin-1-yl)phenyl)amino)-6-methylpyrido[2,3-d]pyrimidin-7(8H)-one. Replacement of cyclopentanamine with cyclohexanamine produced the targeted compound 8-cyclohexyl-2-((2-methoxy-4-(4-methylpiperazin-1-yl)phenyl)amino)pyrido[2,3-d]pyrimidin-7(8H)-one.

The compounds of this disclosure can be prepared as illustrated by the exemplary reaction in Scheme 3. Reaction of 5-bromo-2,4-dichloropyrimidine and cyclopentanamine under the catalysis of triethylamine produced 5-bromo-2-chloro-N-cyclopentylpyrimidin-4-amine. Heck reaction of 5-bromo-2-chloro-N-cyclopentylpyrimidin-4-amine and ethyl acrylate under the catalysis of (PhCN)₂PdCl₂ produced ethyl-3-(2-chloro-4-(cyclopentylamino)pyrimidin-5-yl)acrylate. Buchwald-Hartwig coupling of ethyl-3-(2-chloro-4-(cyclopentylamino)pyrimidin-5-yl)acrylate and 2-methoxy-4-(4-methylpiperazin-1-yl)aniline under the catalysis of (Pd(OAc)₂ produced ethyl-3-(4-(cyclopentylamino)-2-((2-methoxy-4-(4-methylpiperazin-1-yl)phenyl)amino)pyrimidin-5-yl)acrylate. Reduction of ethyl-3-(4-(cyclopentylamino)-2-((2-m ethoxy-4-(4-methylpiperazin-1-yl)phenyl)amino)pyrimidin-5-yl)acrylate with Pd/C/H₂ produced ethyl 3-(4-(cyclopentylamino)-2-((2-methoxy-4-(4-methylpiperazin-1-yl)phenyl)amino)pyrimidin-5-yl)propanoate. Hydrolysis of ethyl 3-(4-(cyclopentylamino)-2-((2-m ethoxy-4-(4-methylpiperazin-1-yl)phenyl)amino)pyrimidin-5-yl)propanoate under the catalysis of sodium hydroxide produced 3-(4-(cyclopentylamino)-2-((2-methoxy-4-(4-methylpiperazin-1-yl)phenyl)amino)pyrimidin-5-yl)propanoic acid. Reaction of 3-(4-(cyclopentylamino)-2-((2-methoxy-4-(4-methylpiperazin-1-yl)phenyl)amino)pyrimidin-5-yl)propanoic acid and 2-(7-azabenzotriazol-1-yl)-N,N,N′,N′-tetramethyluronium hexafluorophosphate (HATU) produced 8-cyclopentyl-2-((2-methoxy-4-(4-methylpiperazin-1-yl)phenyl)amino)-5,8-dihydropyrido[2,3-d]pyrimidin-7(6H)-one.

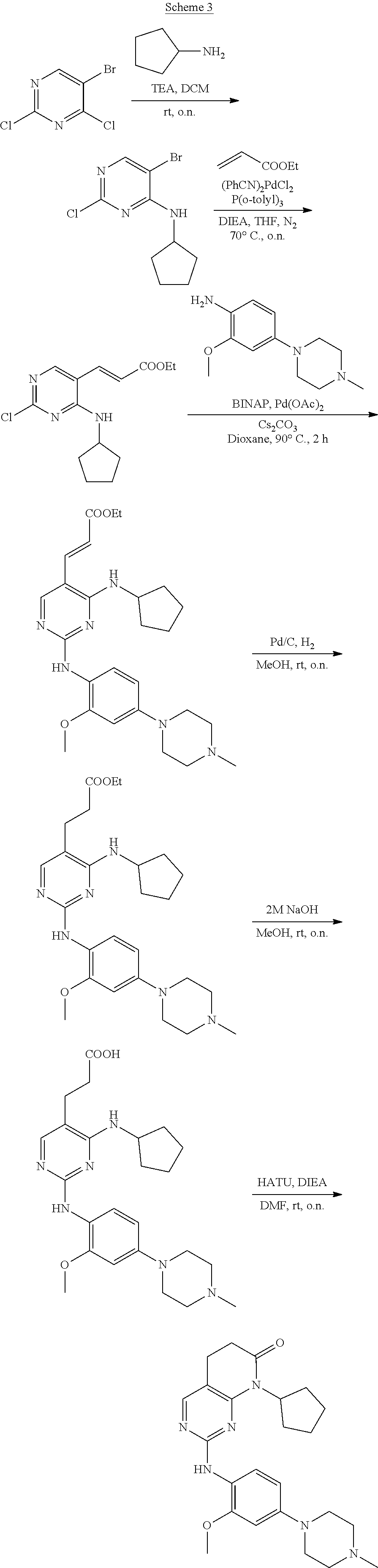

Other related compounds can be prepared using similar methods. For example, replacement of cyclopentanamine with cyclohexanamine produced the targeted compound 8-cyclohexyl-2-((2-methoxy-4-(4-methylpiperazin-1-yl)phenyl)amino)-5,8-dihydropyrido[2,3-d]pyrimidin-7(6H)-one. Replacement of cyclopentanamine with cyclopropanamine produced the targeted compound 8-cyclopropyl-2-((2-methoxy-4-(4-methylpiperazin-1-yl)phenyl)amino)-5,8-dihydropyrido[2,3-d]pyrimidin-7(6H)-one.

The compounds of this disclosure can be prepared as illustrated by the exemplary reaction in Scheme 4. Oxidation of 8-iodo-2-(methylthio)pyrido[4,3-d]pyrimidine with 3-chloroperoxybenzoic acid produced 8-iodo-2-(methylsulfinyl)pyrido[4,3-d]pyrimidine. Reaction of 8-iodo-2-(methylsulfinyl)pyrido[4,3-d]pyrimidine and 2-methoxy-4-(4-methylpiperazin-1-yl)aniline under the catalysis of trifluoroacetic acid produced 8-iodo-N-(2-methoxy-4-(4-methylpiperazin-1-yl)phenyl)pyrido[4,3-d]pyrimidin-2-amine. Suzuki coupling of 8-iodo-N-(2-methoxy-4-(4-methylpiperazin-1-yl)phenyl)pyrido[4,3-d]pyrimidin-2-amine and 2-(cyclopent-1-en-1-yl)-4,4,5,5-tetramethyl-1,3,2-dioxaborolane under the catalysis of Pd(PPh₃)₂Cl₂ produced 8-(cyclopent-1-en-1-yl)-N-(2-methoxy-4-(4-methyl piperazin-1-yl)phenyl)pyrido[4,3-d]pyrimidin-2-amine. Reduction of 8-(cyclopent-1-en-1-yl)-N-(2-methoxy-4-(4-methylpiperazin-1-yl)phenyl)pyrido[4,3-d]pyrimidin-2-amine with 4-methylbenzenesulfonhydrazide produced 8-cyclopentyl-N-(2-methoxy-4-(4-methylpiperazin-1-yl)phenyl)pyrido[4,3-d]pyrimidin-2-amine.

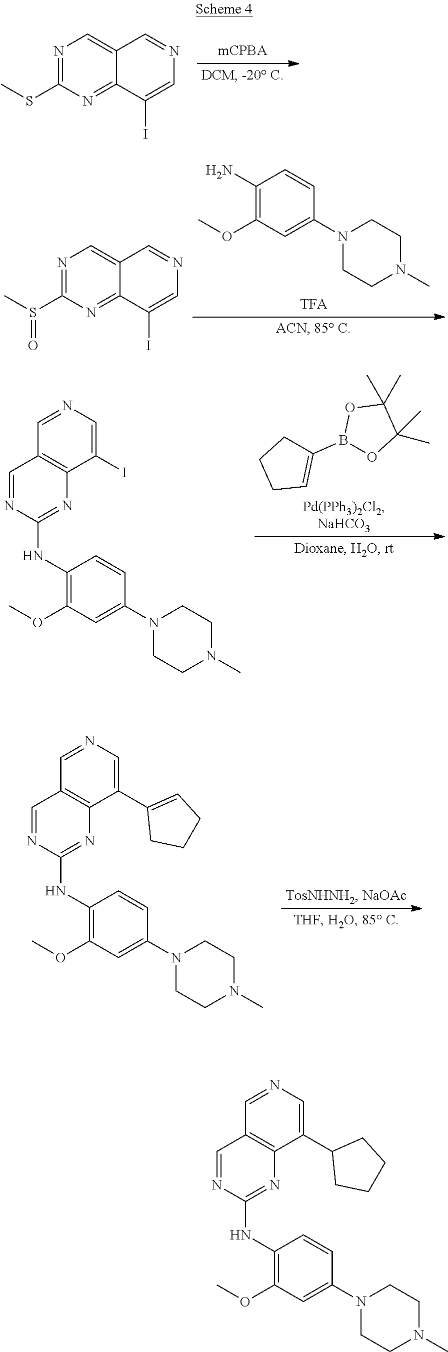

The compounds of this disclosure can be prepared as illustrated by the exemplary reaction in Scheme 5. Bromination of 2-chloro-8-cyclopentyl-5-methylpyrido[2,3-d]pyrimidin-7(8H)-one with NBS under the catalysis of oxalic acid produced 6-bromo-2-chloro-8-cyclopentyl-5-methylpyrido[2,3-d]pyrimidin-7(8H)-one. Stille coupling of 6-bromo-2-chloro-8-cyclopentyl-5-methylpyrido[2,3-d]pyrimidin-7(8H)-one and tributyl (1-ethoxyvinyl)stannane under the catalysis of bis(tri-tert-butylphosphine)palladium, then reacted with hydrochloric acid, produced 6-acetyl-2-chloro-8-cyclopentyl-5-methylpyrido[2,3-d]pyrimidin-7(8H)-one. Buchwald-Hartwig coupling of 6-acetyl -2-chloro-8-cyclopentyl-5-methylpyrido[2,3-d]pyrimidin-7(8H)-one and 2-methoxy-4-(4-methylpiperazin-1-yl)aniline under the catalysis of methanesulfonato [9,9-dimethyl-4,5-bis(diphenylphosphino)xanthene][2′-amino-1,1′-biphenyl]palladium(II) dichloromethane adduct (Pd-G3) produced 6-acetyl-8-cyclopentyl-2-(2-methoxy-4-(4-methylpiperazin-1-yl)phenyl)amino)-5-methylpyrido[2,3-d]pyrimidin-7(8H)-one.

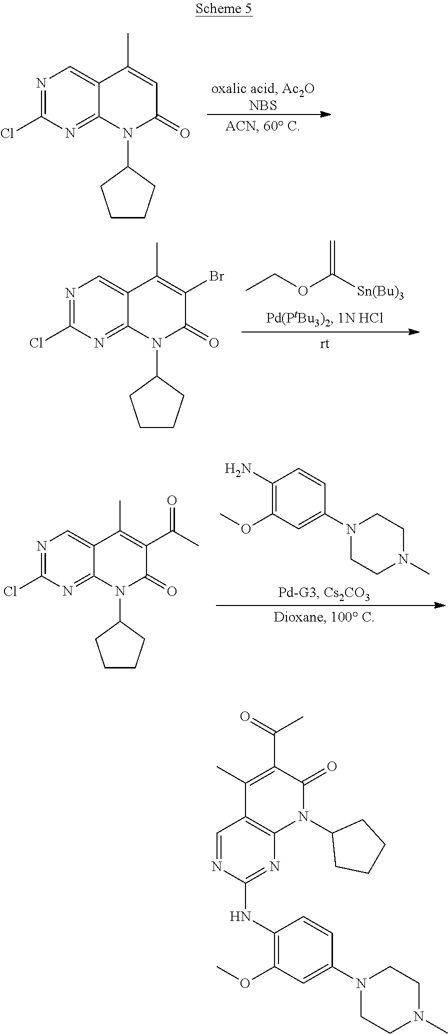

Other related compounds can be prepared using similar methods. For example, replacement of 2-methoxy-4-(4-methylpiperazin-1-yl)aniline with tert-butyl 4-(4-amino-3-methoxyphenyl)piperazine-1-carboxylate produced the targeted compound 6-acetyl-8- cyclopentyl-2-((2-methoxy-4-(piperazin-1-yl)phenyl)amino)-5-methylpyrido[2,3-d]pyrimidin-7(8E1)-one.

An important aspect of the present disclosure is the discovery that compounds of Formula I (including Formulae II and III) are kinase inhibitors, especially NUAK1/2 inhibitors. Therefore, the compound of Formula I (including Formulae II and III) or pharmaceutically acceptable salts thereof, or geometric isomers, enantiomers, diastereomers, racemates, isotope-substituted derivatives, solvates, hydrates or prodrugs thereof can be used as individual active ingredients for the treatment and prevention of NUAK1/2-mediated diseases, disorders and conditions; or for the preparation of drugs for the treatment and prevention of NUAK1/2-mediated diseases, disorders and conditions; it can also be used as a NUAK1/2 inhibitor combination with other anticancer drugs, including but not limited to DSB inducers (such as radiation), topoisomerase II inhibitors (such as etoposide, doxorubicin) and/or PARP inhibitors (such as olaparib, niraparib, rucaparib, talazoparib, pamiparib, fluzoparib and senaparib) for the treatment and prevention of NUAK1/2-mediated diseases, disorders and conditions or for the preparation of treatment and prevention drugs for NUAK1/2 mediated diseases, disorders and conditions.

In the disclosure, NUAK1/2-mediated diseases, disorders and conditions include cancer. Cancer can be a solid tumor or hematological tumor, including but is not limited to liver cancer, melanoma, Hodgkin's disease, non-Hodgkin's lymphoma, acute lymphocytic leukemia, chronic lymphocytic leukemia, multiple myeloma, neuroblastoma, breast cancer, ovarian cancer, lung cancer (such as small cell lung cancer), Wilms tumor, cervical cancer, testicular cancer, soft tissue sarcoma, primary macroglobulinemia, bladder cancer, chronic myeloid leukemia, primary brain cancer, malignant melanoma, gastric cancer, colon cancer, malignant pancreatic islet tumor, malignant carcinoid cancer, choriocarcinoma, mycosis fungoides, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostate cancer. Preferably, the cancer is mediated by NUAK1/2 and related to NUAK1/2; the “mediated” and “associated” mean to play a role in the occurrence and development of cancer, such as leading to the occurrence of cancer, and/or promote the development or metastasis of cancer.

Therefore, the present disclosure includes methods for the treatment or prevention of NUAK1/2-mediated diseases, disorders and conditions, comprising administering to a subject in need thereof an effective amount of the compound of Formula I (including Formulae II and III) or pharmaceutically acceptable salts thereof, or geometric isomers, enantiomers, diastereomers, racemates, isotope-substituted derivatives, solvates, hydrates or prodrugs thereof, or a pharmaceutical composition comprising an effective amount of the compound of Formula I (including Formulae II and III) or pharmaceutically acceptable salts thereof, or geometric isomers, enantiomers, diastereomers, racemates, isotope-substituted derivatives, solvates, hydrates or prodrugs thereof. In the disclosure, subjects include mammals, more specifically humans. In some embodiments, the methods of the present invention for treating or preventing NUAK1/2-mediated diseases, disorders and conditions further comprise simultaneously or sequentially administering to a subject in need thereof a therapeutically effective amount of at least one known anticancer drug or its a pharmaceutically acceptable salt; the at least one known anticancer drug or a pharmaceutically acceptable salt thereof is as described in any embodiment herein.

In practicing the therapeutic methods, effective amounts of pharmaceutical preparations are administered to an individual exhibiting the symptoms of one or more of these disorders. The pharmaceutic preparations comprise a therapeutically effective amount of the compound of Formula I (including Formulae II and III), formulated for oral, intravenous, local or topical application, for the treatment of cancer and other diseases. The amount is effective to ameliorate or eliminate one or more symptoms of the disorders. An effective amount of a compound for treating a particular disease is an amount that is sufficient to ameliorate or in some manner reduce the symptoms associated with the disease. Such amount may be administered as a single dosage or may be administered according to an effective regimen. The amount may cure the disease but, typically, is administered in order to ameliorate the symptoms of the disease. Typically, repeated administration is required to achieve the desired amelioration of symptom.

In another embodiment, there is provided a pharmaceutical composition comprising a compound of Formula I (including Formulae II and III) as a NUAK1/2 inhibitor, or pharmaceutically acceptable salts thereof, or geometric isomers, enantiomers, diastereomers, racemates, isotope-substituted derivatives, solvates, hydrates or prodrugs thereof and pharmaceutically acceptable carriers.

Another embodiment of the present disclosure is directed to a pharmaceutical composition effective to treat or prevent cancer comprising a compound of Formula I (including Formulae II and III) as a NUAK1/2 inhibitor, or pharmaceutically acceptable salts thereof, or geometric isomers, enantiomers, diastereomers, racemates, isotope-substituted derivatives, solvates, hydrates or prodrugs thereof, in combination with at least one known anticancer agent or a pharmaceutically acceptable salt thereof

Herein, the said at least one known anticancer drug or a pharmaceutically acceptable salt thereof includes other anticancer drugs related to DNA damage and repair mechanisms, including PARP inhibitors, such as olaparib, niraparib, rucaparib, talazoparib, pamiparib, fluzoparib and senaparib; HDAC inhibitors such as Vorinostat, Romidepsin, Panobinostat and Belinostat; and so on. The said at least one known anticancer drug or a pharmaceutically acceptable salt thereof also includes other anticancer drugs related to cell division checkpoints, including CDK4/6 inhibitors such as Palbociclib, ATM inhibitors, Weel inhibitors, Myt1 inhibitors, DNA-PK inhibitors, and so on. And combination with other targeted anti-cancer drugs, including USP1 inhibitors, PRMT5 inhibitors, Pole inhibitors, RAD₅₁ inhibitors, and so on. Other known anticancer agents which may be used for anticancer combination therapy include, but are not limited to alkylating agents, such as busulfan, melphalan, chlorambucil, cyclophosphamide, ifosfamide, temozolomide, bendamustine, cis-platin, mitomycin C, bleomycin and carboplatin; topoisomerase I inhibitors, such as camptothecin, irinotecan and topotecan; topoisomerase II inhibitors, such as doxorubicin, epirubicin, aclacinomycin, mitoxantrone, elliptinium and etoposide; RNA/DNA antimetabolites, such as 5-azacytidine, gemcitabine, 5-fluorouracil, capecitabine and methotrexate; DNA antimetabolites, such as 5-fluoro-2′-deoxy-uridine, fludarabine, nelarabine, ara-C, pralatrexate, pemetrexed, hydroxyurea and thioguanine; antimitotic agent such as colchicine, vinblastine, vincristine, vinorelbine, paclitaxel, ixabepilone, cabazitaxel and docetaxel; antibodies such as mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, Ofatumumab, Dinutuximab, Blinatumomab, ipilimumab, avastin, herceptin and mabthera; Antibody-Drug Conjugates (ADC) such as T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin; kinase inhibitors such as imatinib, gefitinib, erlotinib, osimertinib, afatinib, ceritinib, alectinib, crizotinib, erlotinib, lapatinib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, Idelali sib, pazopanib, Torisel and everolimus. Other known anticancer agents which may be used for anticancer combination therapy include tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin (recombinant human interleukin-2) and Sipueucel-T (prostate cancer treatment vaccine).

In practicing the methods of the present disclosure, the compound of the disclosure may be administered together with at least one known anticancer agent in a unitary pharmaceutical composition. Alternatively, the compound of the disclosure may be administered separately from at least one known anticancer agent. In one embodiment, the compound of the disclosure and at least one known anticancer agent are administered substantially simultaneously, i.e. all agents are administered at the same time or one after another, provided that compounds reach therapeutic levels in the blood at the same time. In another embodiment, the compound of the disclosure and at least one known anticancer agent are administered according to individual dose schedule, provided that the compounds reach therapeutic levels in the blood.

Another embodiment of the present disclosure is directed to a bioconjugate, which functions as a kinase inhibitor, that comprises a compound described herein and is effective to inhibit tumor. The bioconjugate that inhibits tumor is consisted of the compound described herein and at least one known therapeutically useful antibody, such as trastuzumab or rituximab, or growth factor, such as EGF or FGF, or cytokine, such as IL-2 or IL-4, or any molecule that can bind to cell surface. The antibodies and other molecules could deliver the compound described herein to its targets, making it an effective anticancer agent. The bioconjugates could also enhance the anticancer effect of the therapeutically useful antibodies, such as trastuzumab or rituximab.

Another embodiment of the present disclosure is directed to a pharmaceutical composition effective to inhibit tumor comprising the NUAK1/2 inhibitor of Formula I (including Formulae II and III), or pharmaceutically acceptable salts thereof, or geometric isomers, enantiomers, diastereomers, racemates, isotope-substituted derivatives, solvates, hydrates or prodrugs thereof, in combination with radiation therapy. In this embodiment, the compound of the disclosure may be administered at the same time as the radiation therapy or at a different time.

Yet another embodiment of the present disclosure is directed to a pharmaceutical composition effective for post-surgical treatment of cancer, comprising the NUAK1/2 inhibitor of Formula I (including Formulae II and III) or pharmaceutically acceptable salts thereof, or geometric isomers, enantiomers, diastereomers, racemates, isotope-substituted derivatives, solvates, hydrates or prodrugs thereof. The disclosure also relates to a method of treating cancer by surgically removing tumor and then treating the mammal with the pharmaceutical composition described herein.

Pharmaceutical compositions of this disclosure include all pharmaceutical preparations which contain the compounds of the present disclosure in an amount that is effective to achieve its intended purpose. While individual needs vary, determination of optimal amounts of each component in the pharmaceutical preparations is within the skill of the art. Typically, the compounds or the pharmaceutically acceptable salt thereof may be administered to mammals, orally at a dose of about 0.0025 to 50 mg per kg body weight per day. Preferably, from approximately 0.01 mg/kg to approximately 10 mg/kg body weight is orally administered. If a known anticancer agent is also administered, it is administered in an amount that is effective to achieve its intended purpose. The optimal amounts of such known anticancer agents are well known to those skilled in the art.

The unit oral dose may comprise from approximately 0.01 to approximately 50 mg, preferably approximately 0.1 to approximately 10 mg of the compound of the disclosure. The unit dose may be administered one or more times, with one or more tablets daily, each containing from approximately 0.1 to approximately 50 mg, conveniently approximately 0.25 to 10 mg of the compound of the disclosure or its solvates.

In a topical formulation, the compound of the disclosure may be present at a concentration of approximately 0.01 to 100 mg per gram of carrier.

The compound of the disclosure may be administered as a raw chemical. The compounds of the disclosure may also be administered as part of a suitable pharmaceutical preparation containing pharmaceutically acceptable carriers (comprising excipients and auxiliaries), which facilitate the processing of the compounds into pharmaceutically acceptable preparations. Preferably, the pharmaceutical preparations, particularly oral preparations and those used for the preferred administration, such as tablets, draggers, and capsules, as well as solutions suitable for injection or oral administration, contain from approximately 0.01% to 99%, preferably from approximately 0.25% to 75% of active compound(s), together with excipient(s).

Also included within the scope of the present disclosure are the non-toxic pharmaceutically acceptable salts of the compounds of the present disclosure. Acid addition salts are formed by mixing a solution of the compounds of the present disclosure with a solution of a pharmaceutically acceptable non-toxic acid, such as hydrochloric acid, fumaric acid, maleic acid, succinic acid, acetic acid, citric acid, tartaric acid, carbonic acid, phosphoric acid, oxalic acid, and the like. Base addition salts are formed by mixing a solution of the compounds of the present disclosure with a solution of a pharmaceutically acceptable non-toxic base, such as sodium hydroxide, potassium hydroxide, choline hydroxide, sodium carbonate, tris(hydroxymethyl)aminomethane, N-methyl-glutamine and the like.

The pharmaceutical preparations of the disclosure may be administered to any mammal, so long as they may experience the therapeutic effects of the compounds of the disclosure. Foremost among such mammals are humans and veterinary animals, although the disclosure is not intended to be so limited.

The pharmaceutical preparations of the present disclosure may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively or concurrently, administration may be by oral route. The dosage administered will be dependent upon the age, health, and weight of the recipient, type of concurrent treatment, frequency of treatment, and the nature of the effect desired.

The pharmaceutical preparations of the present disclosure are manufactured in a known manner, e.g., by means of conventional mixing, granulating, dragee-making, dissolving, or lyophilizing processes. Pharmaceutical preparations for oral use may be obtained by combining the active compounds with solid excipients, optionally grinding the resulting mixture, processing the mixture of granules after adding suitable auxiliaries if desired or necessary, thereby obtaining tablets or dragee cores.

Suitable excipients are, in particular, fillers, such as saccharides, e.g. lactose or sucrose, mannitol or sorbitol; cellulose preparations and/or calcium phosphates, e.g. tricalcium phosphate or calcium hydrogen phosphate; as well as binders, such as starch paste, including, e.g., maize starch, wheat starch, rice starch, potato starch, gelatin, tragacanth, methylcellulose, hydroxypropyl methylcellulose, sodium carboxymethylcellulose, and/or polyvinyl pyrrolidone. If desired, disintegrating agents may be added, such as the above-mentioned starches and also carboxymethyl-starch, cross-linked polyvinyl pyrrolidone, agar, or alginic acid or a salt thereof, such as sodium alginate. Auxiliaries are, in particular, flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetyl cellulose phthalate or hydroxypropyl methylcellulose phthalate, are used. Dyes or pigments may be added to the tablets or dragee coatings, e.g., for identification or in order to characterize combinations of active compound doses.

Other pharmaceutical preparations, which may be used orally, include push-fit capsules made of gelatin, as well as soft, sealed capsules made of gelatin and a plasticizer, such as glycerol or sorbitol. The push-fit capsules may contain the active compounds in the form of granules, which may be mixed with fillers, such as lactose; binders, such as starches; and/or lubricants, such as talc or magnesium stearate and stabilizers. In soft capsules, the active compounds are preferably dissolved or suspended in suitable liquids, such as fatty oils, or liquid paraffin. In addition, stabilizers may be added.

Suitable formulations for parenteral administration include aqueous solutions of the active compounds, e.g., aqueous solutions and alkaline solutions of water-soluble salts. In addition, suspensions of the active compounds as appropriate oily injection suspensions may be administered. Suitable lipophilic solvents or vehicles include fatty oils, e.g., sesame oil, or synthetic fatty acid esters, e.g., ethyl oleate or triglycerides or polyethylene glycol-400, or cremophor, or cyclodextrins. Aqueous injection suspensions may contain substances which increase the viscosity of the suspension, e.g., sodium carboxymethyl cellulose, sorbitol, and/or dextran. Optionally, suspension stabilizers may also be contained.

In accordance with one aspect of the present disclosure, compounds of the disclosure are employed in topical and parenteral formulations and are used for the treatment of skin cancer.

The topical formulations of this disclosure are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than Cu). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The present disclosure also involves use of the compounds of the disclosure for the preparation of medicaments for the treatment and prevention of NUAK1/2-mediated diseases, disorders and clinical conditions. These medicaments may include the above-mentioned pharmaceutical compositions.

The following examples are illustrative, but not limiting, of the method and compositions of the present disclosure. Other suitable modifications and adaptations of the variety of conditions and parameters normally encountered in clinical therapy and which are obvious to those skilled in the art are within the spirit and scope of the disclosure.

### EXAMPLES

### General Remarks

All reagents were of commercial quality. Solvents were dried and purified by standard methods. Mass spectrum analyses were recorded on a Platform II (Agilent 6110) quadrupole mass spectrometer fitted with an electrospray interface. ¹H NMR spectra was recorded at 400 MHz, on a Brucker Ascend 400 apparatus. Chemical shifts were recorded in parts per million (ppm) downfield from TMS (0.00 ppm), and J coupling constants were reported in hertz (Hz).

### Example 1

### 8-cyclopentyl-((2-methoxy-4-(4-methylpiperazin-1-yl)phenyl)amino)pteridin-7(8H)-one

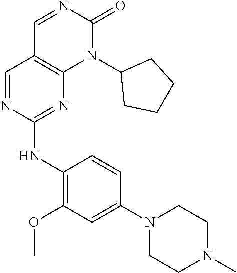 [SMILES: COc1cc(N2CCN(C)CC2)ccc1Nc1ncc2cnc(=O)n(C3CCCC3)c2n1] [0169] a) Preparation of 2-chloro-N-cyclopentyl-5-nitropyrimidin-4-amine: To a solution of 2,4-dichloro-5-nitropyrimidine (2.0 g, 10.3 mmol, 1.0 eq) and cyclopentamine (0.88 g, 10.3 mmol, 1.0 eq) in dichloromethane (20 mL) was added DIEA (2.5 g, 19.1 mmol, 1.8 eq) at −78° C. After stirred at −78° C. for 2 hours, the reaction solution was concentrated under reduce pressure. The residue was washed with water (20 mL) to afford the product (2.3 g, yield: 92%, white solid). LC-MS: 243.40 [M−1]⁺.
b) Preparation of N⁴-cyclopentyl-N²-(2-methoxy-4-(4-methylpiperazin-1-yl)phenyl)-5-nitropyrimidine-2,4-diamine: To a solution of 2-chloro-N-cyclopentyl-5-nitropyrimidin-4-amine (1.0 g 4.13 mmol, 1.0 eq) and 2-methoxy-4-(4-methylpiperazin-1-yl)aniline (1.1 g, 4.9 mmol, 1.2 eq) in THF (10 mL) was added DIEA (0.8 g, 6.2 mmol, 1.5 eq). After stirred at room temperature for 3 hours, to the mixture was added water (20 mL) and the mixture was extracted with EA (20 mL×3). The organic phase was washed with brine, dried over anhydrous sodium sulfate and concentrated under reduce pressure. The residue was purified by chromatography over silica gel (DCM: MeOH=1:0˜50:1) to afford the product (1.3 g, yield: 74%, red solid). LC-MS: 428.35 [M+1]⁺.
c) Preparation of N⁴-cyclopentyl-N²-(2-methoxy-4-(4-methylpiperazin-1-yl)phenyl)pyrimidine-2,4,5-triamine: To a solution of N⁴-cyclopentyl-N²-(2-methoxy-4-(4-methylpiperazin-1-yl)phenyl)-5-nitropyrimidine-2,4-diamine (0.3 g, 0.7 mmol, 1.0 eq) in MeOH (3 mL) was added Pd/C (0.06 g, 20% W/W). After stirred at room temperature for 3 hours under H₂, the mixture was filtered and the cake was washed with MeOH (10 mL×3). The organic phase was concentrated under reduce pressure to afford the product (0.27 g, yield: 96%, black solid). LC-MS: 398.20 [M+1]⁺.
d) Preparation of 8-cyclopentyl-2-((2-methoxy-4-(4-methylpiperazin-1-yl)phenyl)amino)pteridin-7(8H)-one: To a solution of N⁴-cyclopentyl-N²-(2-methoxy-4-(4-methylpiperazin-1-yl)phenyl)pyrimidine-2,4,5-triamine (0.08 g, 0.2 mmol, 1.0 eq) and ethyl 2-oxoacetate (0.02 g, 0.4 mmol, 2.0 eq) in EtOH (6 mL) was added AcOH (0.2 mL). The mixture was refluxed at 80° C. for 48 h. After completion of the reaction, water (10 mL) was added and the mixture was extracted with EA (10 mL×3). The combined organic phase was washed with brine, dried over anhydrous sodium sulfate and concentrated under reduce pressure. The residue was triturated with petroleumether: ethyl acetate=10:1 (5 mL×2) to afford the target compound (0.01 g, yield: 11%, yellow solid). LC-MS: 436.45 [M+1]⁺. ¹H NMR (400 MHz, CD₃OD): δ 8.66 (s, 1H), 7.79 (s, 1H), 7.71-7.59 (m, 1H), 6.69 (s, 1H), 6.58 (d, J=8.9 Hz, 1H), 5.66 (t, J=10.5 Hz, 1H), 3.84 (s, 3H), 2.80 (s, 4H), 2.48 (s, 3H), 2.31-2.20 (m, 2H), 2.14 (s, 2H), 1.94 (s, 2H), 1.79 (t, J=12.2 Hz, 4H), 1.65-1.48 (m, 2H).

### Example 2

### 1-cyclopentyl-7-((2-methoxy-4-(4-methylpiperazin-1-yl)phenyl)amino)-3,4-dihydropyrimido[4,5-d]pyrimidin-2(1H)-one

a) Preparation of ethyl 4-(cyclopentylamino)-2-(methylthio)pyrimidine-5-carboxylate: To a solution of ethyl 4-chloro-2-(methylthio)pyrimidine-5-carboxylate (30.0 g, 128.9 mmol, 1.0 eq) and TEA (26.0 g, 257.8 mmol, 2.0 eq) in dichloromethane (300 mL) was added cyclopentamine (12.1 g, 141.8 mmol, 1.1 eq) at 0° C. After stirred at 0° C. for 30 min, the solution was warmed to room temperature and stirred overnight. The mixture was quenched with water (200 mL) and the organic phase was washed with brine, dried over anhydrous sodium sulfate and concentrated under reduce pressure to afford the product (35.0 g, yield: 97%, colorless oil). LC-MS: 282.35 [M+1]⁺.
b) Preparation of (4-(cyclopentylamino)-2-(methylthio)pyrimidin-5-yl)methanol: To a solution of lithium aluminum hydride (1.0 g, 26.6 mmol, 1.5 eq) in THF (50 mL) was added ethyl 4-(cyclopentylamino)-2-(methylthio)pyrimidine-5-carboxylate (5.0 g, 19.8 mmol, 1.0 eq) at 0° C. After stirred at 0° C. for 30 min, the solution was warmed to room temperature and stirred for 5 h. To the mixture was added water (20 mL) and 15% aqueous sodium hydroxide (10 mL) at 0° C., and then stirred for 15 min. The mixture was filtered and the cake was washed with DCM (50 mL×3). The organic phase was washed with brine, dried over anhydrous sodium sulfate and concentrated under reduce pressure to afford the product (2.4 g, yield: 50%, green solid). LC-MS: 240.35[M+1]⁺.
c) Preparation of 5-(chloromethyl)-N-cyclopentyl-2-(methylthio)pyrimidin-4-amine: A solution of (4-(cyclopentylamino)-2-(methylthio)pyrimidin-5-yl)meth anol (10.0 g, 41.8 mmol, 1.0 eq) in thionyl chloride (100 mL) was refluxed at 100° C. for 1 h. After completion of the reaction, the mixture was concentrated under reduced pressure to afford the product (crude, 10.0 g), which was used for the next step directly without further purification.
d) Preparation of 5-(aminomethyl)-N-cyclopentyl-2-(methylthio)pyrimidin-4-amine: To a solution of 5-(chloromethyl)-N-cyclopentyl-2-(methylthio)pyrimidin-4-amine (crude, 10.0 g) in THF (100 mL) was bubbled ammonia gas for 3 min at −78° C. The mixture was stirred at −78° C. for 30 min, and then warmed to room temperature and stirred overnight. After completion of the reaction, the mixture was concentrated under reduced pressure. The residue was purified by chromatography over silica gel (petroleumether: ethyl acetate=1:2-0:1) to afford the product (6.8 g, 2 steps yield: 68%, yellow oil). LC-MS: 239.40 [M+1]⁺.
e) Preparation of 1-cyclopentyl-7-(methylthio)-3,4-dihydropyrimido[4,5-d]pyrimidin-2: To a solution of 5-(aminomethyl)-N-cyclopentyl-2-(methylthio)pyrimidin-4-amine (1.1 g, 4.8 mmol, 1.0 eq) in THF (11 mL) was added 1,1′-carbonyldiimidazole (0.9 g, 5.7 mmol, 1.2 eq) at 0° C. The mixture was stirred at 0° C. for 30 min, and then stirred at 75° C. overnight in a sealed tube. After completion of the reaction, to the mixture was added EA (30 mL) and the mixture was washed with saturated aqueous sodium bicarbonate and brine. The organic phase was separated and dried, concentrated to give the residue which was purified by chromatography over silica gel (petroleumether: ethyl acetate=10:1˜1:1) to afford the product (0.4 g, yield: 35%, white solid). LC-MS: 265.30 [M−1]⁺. ¹H NMR (400 MHz, Methanol-d₄): δ 8.09 (s, 1H), 5.29-5.14 (m, 1H), 4.27 (s, 2H), 2.52 (s, 3H), 2.28-2.13 (m, 2H), 2.01-1.91 (m, 2H), 1.85 (dd, J=19.1, 10.1 Hz, 2H), 1.61 (dq, J=10.5, 6.5, 6.0 Hz, 2H).
f) Preparation of 1-cyclopentyl-7-(methylsulfinyl)-3,4-dihydropyrimido[4,5-d]pyrimidin-2(1H)-one: To a solution of 1-cyclopentyl-7-(methylthio)-3,4-dihydropyrimido[4,5-d]pyrimidin-2(1H)-one (0.3 g, 1.13 mmol, 1.0 eq) in DCM (6 mL) was added 3-chloroperoxybenzoic acid (0.13 g, 1.25 mmol, 1.1 eq) at 0° C. The mixture was stirred at 0° C. for 2 h. After completion of the reaction, the mixture was quenched with aqueous sodium thiosulfate (5 mL) and the organic phase was washed with aqueous saturated sodium bicarbonate. The mixture was extracted with DCM (10 mL×3). The combined organic phase was washed with brine, dried over anhydrous sodium sulfate and concentrated under reduce pressure to afford the product (crude, 0.2 g), which was used for the next step directly without further purification. LC-MS: 281.05 [M+1]⁺, 279.05[M−1]⁻.
g) Preparation of 1-cyclopentyl-7-((2-methoxy-4-(4-methylpiperazin-1-yl)phenyl)amino)-3,4-dihydropyrimido[4,5-d]pyrimidin-2(1H)-one: To a solution of 1-cyclopentyl-7-(methylsulfinyl)-3,4-dihydropyrimido[4,5-d]pyrimidin-2(1H)-one (40.0 mg, 0.14 mmol, 1.0 eq) and 2-methoxy-4-(4-methylpiperazin-1-yl)aniline (50.0 mg, 0.21 mmol, 1.5 eq) in ACN (1 mL) was added trifluoroacetic acid (120.0 mg, 1.1 mmol, 5.0 eq). The mixture was stirred at 85° C. overnight. After completion of the reaction, DCM (10 mL) was added and the mixture was washed with aqueous sodium bicarbonate (10 mL). The combined organic phase was washed with brine, dried over anhydrous sodium sulfate and concentrated under reduce pressure. The residue was purified by Prep-TLC (DCM: MeOH=10:1) to afford the target compound (13.0 mg, 2 steps yield: 20%, green solid).

The compounds of Examples 3-4 were prepared using a synthesis method similar to that described in Example 2.

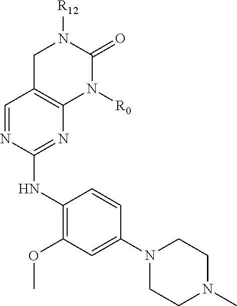

| Example | R₀ | R₁₂ | MW | LC-MS | ¹H NMR (400 MHz) |
|---|---|---|---|---|---|
| 2 | 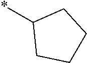 [SMILES: *C1CCCC1] | H | 437.55 | 438.20 [M + 1]⁺ | CD₃OD: δ 7.91 (s, 1H), 7.75 (d, J = 8.4 Hz, 1H), 6.66 (s, 1H), 6.53 (d, J = 7.8 Hz, 1H), 5.17-5.07 (m, 1H), 4.19 (s, 2H), 3.85 (s, 3H), 3.21-3.16 (m, 4H), 2.66-2.59 (m, 4H), 2.35 (s, 3H), 2.24- |
| | | | | | 2.12 (m, 2H), 1.77 (brs, 4H), 1.52 (brs, 2H). |
| 3 | 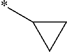 [SMILES: *C1CC1] | H | 409.49 | 410.20 [M + 1]⁺ | CDCl₃: δ 8.48 (d, J = 8.5 Hz, 1H), 7.95 (s, 1H), 7.53 (s, 1H), 6.55 (s, 2H), 5.23 (s, 1H), 4.23 (brs, 2H), 3.89 (s, 3H), 3.38-3.17 (m, 4H), 2.88- |
| | | | | | 2.66 (m, 5H), 2.49 (s, 3H), 1.22-1.10 (m, 2H), |
| | | | | | 0.88-0.70 (m, 2H). |
| 4 | 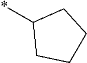 [SMILES: *C1CCCC1] | CH₃ | 451.58 | 452.20 [M + 1]⁺ | CD₃OD: δ 7.92 (s, 1H), 7.76 (d, J = 8.3 Hz, 1H), 6.66 (s, 1H), 6.53 (d, J = 8.2 Hz, 1H), 5.16 (t, J = 8.1 Hz, 1H), 4.24 (s, 2H), 3.85 (s, 3H), 3.22-3.15 (m, 4H), 2.97 (s, 3H), 2.66-2.60 (m, 4H), 2.36 |
| | | | | | (s, 3H), 2.16-2.07 (m, 2H), 1.82-1.74 (m, 4H), |
| | | | | | 1.56-1.50 (m, 2H). |

### Example 5

### 1-cyclopentyl-7-((2-methoxy-4-(4-methylpiperazin-1-yl)phenyl)amino)pyrimido[4,5-d]pyrimidin-2(1H)-one

To a solution of 1-cyclopentyl-7-((2-methoxy-4-(4-methylpiperazin-1-yl)phenyl)amino)-3,4-dihydropyrimido[4,5-d]pyrimidin-2(1H)-one (Example 2, 220.0 mg, 0.5 mmol, 1.0 eq) in THF (3 mL) was added potassium tert-butoxide (560.0 mg, 5.02 mmol, 10.0 eq). The mixture was refluxed at 90° C. for 6 h. After completion of the reaction, water (5 mL) was added and the mixture was extracted with EA (5 mL×3). The combined organic phase was washed with brine, dried over anhydrous sodium sulfate and concentrated under reduced pressure. The residue was triturated with diethyl ether and purified by Prep-HPLC (C18, CH₃CN/H₂O, 15˜45%, 0.1% HCOOH added) to afford the target compound (25.0 mg, yield: 12%, yellow solid).

The compounds of Examples 6-19 were prepared using a synthesis method similar to those described in Example 2 and 5.

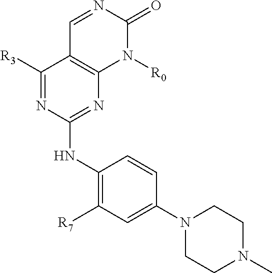

| Example | R₀ | R₃ | R₇ | MW | LC-MS | ¹H NMR (400 MHz) |
|---|---|---|---|---|---|---|
| 5 | 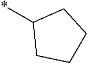 [SMILES: *C1CCCC1] | H | OCH₃ | 435.53 | 436.40 [M + 1]⁺, 434.35 [M − 1]⁻ | CD₃OD: δ 8.50-8.42 (m, 1H), 8.19-8.13 (m, 1H), 7.79-7.73 (m, 1H), 6.72-6.69 (m, 1H), 6.60-6.55 (m, 1H), 5.37 (s, 1H), 5.32-5.25 (m, 1H), 3.86 (s, 3H), 3.37- |
| | | | | | | 3.32 (m, 4H), 3.16-3.11 (m, 4H), 2.73 (s, |
| | | | | | | 3H), 2.27-2.06 (m, 2H), 1.80-1.72 (m, |
| | | | | | | 4H), 1.55-1.47 (m, 2H). |
| 6 | 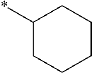 [SMILES: *C1CCCCC1] | H | OCH₃ | 449.56 | 450.40 [M + 1]⁺ | CD₃OD: δ 8.13 (s, 1H), 7.70 (d, J = 8.7 Hz, 1H), 6.67 (s, 1H), 6.55 (d, J = 9.1 Hz, 1H), 5.37 (s, 1H), 4.71 (t, J = 12.2 Hz, 1H), 3.85 (s, 3H), 3.27-3.15 (m, 4H), 2.62 (t, J = 4.8 Hz, 4H), 2.50-2.27 (m, 5H), 1.78 (d, J = |
| | | | | | | 12.9 Hz, 2H), 1.58 (s, 2H), 1.39-1.22 (m, |
| | | | | | | 3H), 1.19-0.99 (m, 1H). |
| 7 | 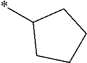 [SMILES: *C1CCCC1] | H | OCH₂CH₃ | 449.55 | 450.35 [M + 1]⁺, 448.30 [M − 1]⁻ | CD₃OD: δ 8.15 (s, 1H), 7.79 (d, J = 8.8 Hz, 1H), 6.67 (s, 1H), 6.56 (d, J = 9.0 Hz, 1H), 5.38 (s, 1H), 5.34-5.23 (m, 1H), 4.09 (q, J = 6.5 Hz, 2H), 3.26 (s, 4H), 2.89 (d, J = |
| | | | | | | 20.0 Hz, 4H), 2.55 (d, J = 14.0 Hz, 3H), |
| | | | | | | 2.28-2.07 (m, 2H), 1.79 (brs, 4H), 1.53 |
| | | | | | | (d, J = 5.0 Hz, 2H), 1.39 (t, J = 6.8 Hz, 3H). |
| 8 | 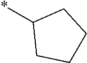 [SMILES: *C1CCCC1] | H | OCH(CH₃)₂ | 463.58 | 464.35 [M + 1]⁺, 462.25 [M − 1]⁻ | CD₃OD: δ 8.17 (s, 1H), 7.84 (d, J = 8.4 Hz, 1H), 6.69 (s, 1H), 6.58 (d, J = 8.9 Hz, 1H), 5.40 (s, 1H), 5.36-5.26 (m, 1H), 4.68-4.56 (m, 1H), 3.21 (brs, 4H), 2.74 (brs, 4H), |
| | | | | | | 2.43 (d, J = 5.7 Hz, 3H), 2.31-2.11 (m, |
| | | | | | | 2H), 1.82 (brs, 4H), 1.56 (d, J = 5.2 Hz, |
| | | | | | | 2H), 1.33 (d, J = 6.5 Hz, 6H). |
| 9 | 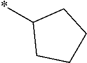 [SMILES: *C1CCCC1] | H | OCF₃ | 489.50 | 490.40 [M + 1]⁺ | CD₃OD: δ 8.16 (s, 1H), 7.63 (d, J = 8.8 Hz, 1H), 7.05 (dd, J = 9.0, 2.7 Hz, 1H), 7.00 (s, 1H), 5.39 (s, 1H), 5.21 (p, J = 8.5 Hz, 1H), 3.50-3.38 (m, 4H), 3.38-3.31 (m, 2H), |
| | | | | | | 2.85 (s, 3H), 2.22-2.10 (m, 1H), 2.13- |
| | | | | | | 1.98 (m, 1H), 1.77-1.67 (m, 2H), 1.66- |
| | | | | | | 1.56 (m, 2H), 1.50-1.40 (m, 2H), 1.37- |
| | | | | | | 1.22 (m, 2H). |
| 10 | 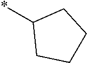 [SMILES: *C1CCCC1] | H | CH₃ | 419.52 | 420.40 [M + 1]⁺, 418.30 [M − 1]⁻ | CD₃OD: δ 8.08 (s, 1H), 7.18 (d, J = 8.6 Hz, 1H), 6.96-6.81 (m, 2H), 5.35 (d, J = 1.8 Hz, 1H), 5.22-5.07 (m, 1H), 3.29-3.26 (m, 4H), 2.98-2.89 (m, 4H), 2.58 (d, J = |
| | | | | | | 12.5 Hz, 3H), 2.21 (d, J = 5.8 Hz, 3H), |
| | | | | | | 2.14-1.99 (m, 2H), 1.70-1.43 (m, 4H), |
| | | | | | | 1.42-1.31 (m, 2H). |
| 11 | 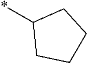 [SMILES: *C1CCCC1] | H | Cl | 439.95 | 440.25 [M + 1]⁺, 438.20 [M − 1]⁻ | DMSO-d₆: δ 9.98 (s, 1H), 8.90 (d, J = 16.2 Hz, 1H), 8.28 (s, 1H), 7.25 (d, J = 8.6 Hz, 1H), 7.10-6.87 (m, 2H), 5.23 (s, 1H), 3.18 (s, 4H), 2.44 (s, 4H), 2.22 (s, 3H), 2.11- |
| | | | | | | 1.90 (m, 2H), 1.68-1.44 (m, 2H), 1.28 |
| | | | | | | (brs, 4H). |
| 12 | 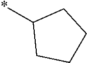 [SMILES: *C1CCCC1] | H | Br | 484.40 | 484.30 [M + 1]⁺ | CD₃OD: δ 8.48 (s, 1H), 8.14 (s, 1H), 7.48 (d, J = 8.8 Hz, 1H), 7.26 (d, J = 2.7 Hz, 1H), 7.01 (dd, J = 8.8, 2.8 Hz, 1H), 5.38 (s, 1H), 5.17 (p, J = 8.7 Hz, 1H), 3.30-3.26 (m, |
| | | | | | | 4H), 2.89-2.82 (m, 4H), 2.53 (s, 3H), 2.20- |
| | | | | | | 2.04 (m, 2H), 1.74-1.63 (m, 2H), 1.57 (brs, |
| | | | | | | 2H), 1.47-1.36 (m, 2H). |
| 13 | 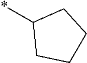 [SMILES: *C1CCCC1] | CH₃ | OCH₃ | 449.55 | 450.20 [M + 1]⁺, 448.10 [M − 1]⁻ | DMSO-d₆: δ 9.35 (s, 1H), 9.07 (s, 1H), 7.20 (d, J = 8.4 Hz, 1H), 6.62 (s, 1H), 6.50 (d, J = 8.5 Hz, 1H), 5.32 (s, 1H), 3.73 (s, 3H), 3.16 (s, 4H), 2.60 (s, 3H), 2.46 (s, 4H), 2.22 |
| | | | | | | (s, 3H), 2.06 (s, 2H), 1.57 (s, 2H), 1.54- |
| | | | | | | 1.21 (m, 4H). |
| 14 | 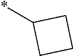 [SMILES: *C1CCC1] | H | OCH₃ | 421.50 | 422.15 [M + 1]⁺, 420.15 [M − 1]⁻ | DMSO-d₆: δ 9.52 (s, 1H), 8.84 (d, J = 15.1 Hz, 1H), 8.26-8.04 (m, 1H), 7.27 (d, J = 9.1 Hz, 1H), 6.62 (d, J = 6.3 Hz, 1H), 6.50 (dd, J = 15.2, 8.7 Hz, 1H), 5.36-5.21 (m, |
| | | | | | | 1H), 3.75 (d, J = 7.6 Hz, 3H), 3.16 (t, J = |
| | | | | | | 5.0 Hz, 4H), 2.87-2.71 (m, 2H), 2.50- |
| | | | | | | 2.44 (m, 4H), 2.23 (s, 3H), 2.05-1.91 (m, |
| | | | | | | 2H), 1.55-1.29 (m, 1H), 1.29-1.17 (m, |
| | | | | | | 1H). |
| 15 | 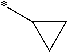 [SMILES: *C1CC1] | H | OCH₃ | 407.48 | 408.35 [M + 1]⁺ | DMSO-d₆: δ 9.15 (s, 1H), 8.87 (d, J = 30.0 Hz, 1H), 8.19-7.96 (m, 1H), 7.94-7.18 (m, 1H), 6.67 (s, 1H), 6.63-6.46 (m, 1H), |
| | | | | | | 4.09 (d, J = 27.1 Hz, 1H), 3.83 (d, J = 9.6 |
| | | | | | | Hz, 3H), 3.25-3.12 (m, 5H), 3.05 (t, J = |
| | | | | | | 7.5 Hz, 2H), 3.00-2.70 (m, 4H), 1.09- |
| | | | | | | 0.91 (m, 2H), 0.78 (brs, 1H), 0.59 (brs, 1 |
| | | | | | | H). |
| 16 | 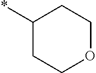 [SMILES: *C1CCOCC1] | H | OCH₃ | 451.53 | 452.30 [M + 1]⁺ | CDCl₃: δ 8.79 (s, 1H), 8.62 (s, 1H), 8.33 (d, J = 8.6 Hz, 1H), 8.13 (s, 1H), 6.60 (d, J = 8.3 Hz, 1H), 6.55 (s, 1H), 5.48-5.40 (m, 1H), 4.15 (d, J = 7.5 Hz, 2H), 3.91 (s, 3H), 3.57-3.48 (m, 2H), 3.25 (s, 4H), 3.17- |
| | | | | | | 3.05 (m, 2H), 2.70-2.60 (m, 4H), 2.39 (s, |
| | | | | | | 3H), 1.61 (d, J = 11.1 Hz, 2H). |
| 17 | 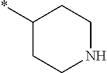 [SMILES: *C1CCNCC1] | H | OCH₃ | 450.55 | — | — |
| 18 | 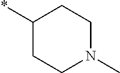 [SMILES: *C1CCN(C)CC1] | H | OCH₃ | 464.57 | — | — |

### Example 19

### 8-cyclopentyl-2-((2-methoxy-4-(4-methylpiperazin-1-yl)amino)pyrido[2,3-d]pyrimidin-7(8H)-one

a) Preparation of 4-(cyclopentylamino)-2-(methylthio)pyrimidine-5-carbaldehyde: A solution of (4-(cyclopentylamino)-2-(methylthio)pyrimidin-5-yl)methanol (Example 2b, 580.0 mg, 2.3 mmol, 1.0 eq) and MnO₂ (1.8 g, 23.1 mmol, 10.0 eq) in CHCl₃ (10 mL) was stirred at room temperature overnight. After completion of the reaction, the reaction mixture was filtered and the filtrate was concentrated under reduced pressure to afford the product (crude, 550.2 mg), which was used for the next step directly without further purification.
b) Preparation of ethyl-3-(4-(cyclopentylamino)-2-(methylthio)pyrimidin-5-yl)acrylate: To a solution of 4-(cyclopentylamino)-2-(methylthio)pyrimidine-5-carbaldehyde (550.2 mg, 2.3 mmol, 1.0 eq) in THF (10 mL) was added ethyl 2-(triphenyl-λ⁵-phosphaneylidene)acetate (960.8 mg, 2.8 mmol, 1.2 eq). The mixture was stirred at 80° C. for 3 h. After completion of the reaction, the mixture was concentrated under reduced pressure. The residue was purified by chromatography over silica gel (petroleumether: ethyl acetate=10:1) to afford the product (710.1 mg, two steps yield: 95%, light yellow solid). LC-MS: 308.15 [M+1]⁺.
c) Preparation of 1-cyclopentyl-7-(methylthio)-3,4-dihydropyrimido[4,5-d]pyrimidin-2(1H)-one: To a solution of ethyl-3-(4-(cyclopentylamino)-2-(methylthio)pyrimidin-5-yl)acrylate (0.7 g, 2.3 mmol, 1.0 eq) in NMP (5 mL) was added DBU (1.0 g, 6.9 mmol, 3.0 eq). The mixture was stirred at 120° C. overnight. After completion of the reaction, to the mixture was added H₂O (10 mL) and the mixture was extracted with EA (10 mL×3). The combined organic phase was washed with brine, dried over anhydrous sodium sulfate. The residue was purified by chromatography over silica gel (petroleumether: ethyl acetate=5:1) to afford the product (0.2 g, yield: 36%, off-white solid). LC-MS: 262.05 [M+1]⁺.
d) Preparation of 8-cyclopentyl-2-(methylsulfinyl)pyrido[2,3-d]pyrimidin-7(8H)-one: To a solution of 8-cyclopentyl-2-(methylthio)pyrido[2,3-d]pyrimidin-7(8H)-one (0.5 g, 1.9 mmol, 1.0 eq) in DCM (10 mL) was added 3-chloroperoxybenzoic acid (0.4 g, 2.1 mmol, 1.1 eq). The mixture was stirred at 0° C. for 2 h. After completion of the reaction, the mixture was quenched with aqueous saturated sodium thiosulfate (10 mL) and washed with saturated aqueous sodium bicarbonate (10 mL). The mixture was extracted with DCM (10 mL×3). The combined organic phase was washed with brine, dried over anhydrous sodium sulfate and concentrated under reduce pressure. The residue was purified by chromatography over silica gel (petroleumether:ethyl acetate=3:1) to afford the product (0.3 g, yield: 52%, light-yellow solid). LC-MS: 278.05 [M+1]⁺. ¹H NMR (400 MHz, CDCl₃): δ 8.58 (s, 1H), 7.57-7.47 (m, 1H), 6.59 (d, J=9.4 Hz, 1H), 5.98-5.85 (m, 1H), 2.67-2.57 (m, 3H), 2.35 (s, 2H), 2.07 (s, 2H), 1.96-1.83 (m, 2H), 1.73-1.65 (m, 2H).
e) Preparation of 8-cyclopentyl-2-((2-methoxy-4-(4-methylpiperazin-1-yl)phenyl)amino)pyrido[2,3-d]pyrimidin-7(8H)-one: To a solution of 8-cyclopentyl-2-(methylsulfinyl)pyrido[2,3-d]pyrimidin-7(8H)-one (93.0 mg, 0.3 mmol, 1.0 eq) and 2-methoxy-4-(4-methylpiperazin-1-yl)aniline (89.0 mg, 0.4 mmol, 1.2 eq) in ACN (10 mL) was added trifluoroacetic acid (0.2 g, 1.7 mmol, 5.0 eq). The mixture was stirred at 85° C. overnight. After completion of the reaction, DCM (20 mL) was added and the mixture was washed with aqueous sodium bicarbonate (10 mL). The combined organic phase was washed with brine, dried over anhydrous sodium sulfate and concentrated under reduce pressure. The residue was purified by Prep-TLC (DCM:MeOH=10:1) to afford the target compound (80.0 mg, yield: 55%, off white solid).

The compounds of Examples 20-24 were prepared using a synthesis method similar to that described in Example 19.

| Example | R₀ | R₁₀ | B₃ | MW | LC-MS | ¹H NMR (400 MHz) |
|---|---|---|---|---|---|---|
| 19 | 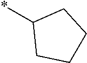 [SMILES: *C1CCCC1] | H | CH | 434.54 | 435.40 [M + 1]⁺ | CDCl₃: δ 8.48 (s, 1H), 8.14 (d, J = 9.2 Hz, 1H), 7.58 (s, 1H), 7.44 (d, J = 9.1 Hz, 1H), 6.57 (s, 2H), 6.38 (d, J = 9.3 Hz, 1H), 5.97-5.84 (m, 1H), 3.90 (s, 3H), 3.30-3.18 |
| | | | | | | (m, 4H), 2.75-2.59 (m, 4H), 2.40 (s, 3H), |
| | | | | | | 2.39-2.30 (m, 2H), 2.02 (t, J = 8.4 Hz, |
| | | | | | | 2H), 1.93-1.80 (m, 2H), 1.72-1.64 (m, |
| | | | | | | 2H). |
| 20 | 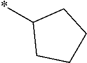 [SMILES: *C1CCCC1] | CH₃ | CH | 448.57 | 449.20 [M + 1]⁺, 447.15 [M − 1]⁻ | CDCl₃: δ 8.43 (s, 1H), 8.17 (d, J = 8.9 Hz, 1H), 7.56 (s, 1H), 7.32 (s, 1H), 6.57 (d, J = 5.1 Hz, 2H), 5.95 (p, J = 8.5 Hz, 1H), 3.90 (s, 3H), 3.25 (t, J = 4.8 Hz, 4H), 2.69 (t, J = |
| | | | | | | 4.8 Hz, 4H), 2.43 (s, 3H), 2.35 (p, J = 8.3, |
| | | | | | | 7.0 Hz, 2H), 2.16 (s, 3H), 2.05 (t, J = 7.5 |
| | | | | | | Hz, 2H), 1.94-1.81 (m, 2H), 1.67 (q, J = |
| | | | | | | 6.0 Hz, 2H). |
| 21 | 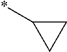 [SMILES: *C1CC1] | CH₃ | CH | 420.53 | 421.20 [M + 1]⁺ | CDCl₃: δ 8.75-8.59 (m, 1H), 8.43 (s, 1H), 7.82 (s, 1H), 7.34 (s, 1H), 6.61 (d, J = 7.1 Hz, 1H), 6.55 (s, 1H), 3.91 (s, 3H), 3.56- 3.42 (m, 4H), 3.27-3.04 (m, 4H), 3.01- |
| | | | | | | 2.94 (m, 1H), 2.75 (s, 3H), 2.17 (s, 3H), |
| | | | | | | 1.42-1.34 (m, 2H), 0.98-0.89 (m, 2H). |
| 22 | 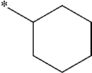 [SMILES: *C1CCCCC1] | H | CH | 448.57 | 449.35 [M + 1]⁺, 447.15 [M − 1]⁻ | CDCl₃: δ 8.45 (s, 1H), 8.23 (s, 1H), 7.86- 7.50 (m, 1H), 7.42 (d, J = 9.4 Hz, 1H), 6.57 (d, J = 6.9 Hz, 2H), 6.37 (d, J = 9.2 Hz, 1H), 5.40 (s, 1H), 3.90 (s, 3H), 3.31 (t, J = 4.9 Hz, 4H), 2.82 (t, J = 4.9 Hz, |
| | | | | | | 4H), 2.75-2.60 (m, 3H), 2.51 (s, 3H), |
| | | | | | | 1.90 (s, 2H), 1.69 (t, J = 19.5 Hz, 3H), |
| | | | | | | 1.41 (t, J = 12.9 Hz, 2H). |
| 23 | 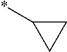 [SMILES: *C1CC1] | H | CH | 406.49 | 407.20 [M + 1]⁺ | CDCl₃: δ 8.74-8.59 (m, 1H), 8.47 (s, 1H), 7.95-7.82 (m, 1H), 7.47 (d, J = 9.2 Hz, 1H), 6.60 (d, J = 8.5 Hz, 1H), 6.56 (s, 1H), 6.40 (d, J = 9.3 Hz, 1H), 3.92 (s, 3H), |
| | | | | | | 3.45-3.28 (m, 4H), 3.18-2.86 (m, 4H), |
| | | | | | | 2.65 (s, 3H), 1.88-1.51 (m, 1H), 1.44-1.33 |
| | | | | | | (m, 2H), 1.01-0.85 (m, 2H). |
| 24 | 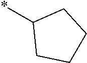 [SMILES: *C1CCCC1] | H | N | 435.53 | 436.15 [M + 1]⁺ | CDCl₃: δ 8.17-8.05 (m, 1H), 8.07-7.87 (m, 2H), 6.69-6.53 (m, 3H), 5.79-5.65 (m, 1H), 3.93 (s, 3H), 3.45-3.28 (m, 4H), 2.91 (s, 4H), 2.58 (s, 3H), 2.36-2.27 (m, |
| | | | | | | 2H), 2.07-1.95 (m, 2H), 1.91-1.84 (m, |
| | | | | | | 2H), 1.67 (s, 2H). |

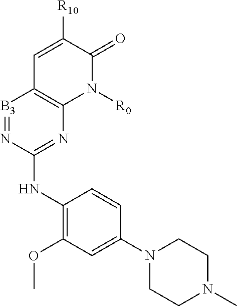

### Example 25

### 1-cyclopentyl-7-((2-methoxy-4-(4-methylpiperazin-1-yl)phenyl)amino)-2-oxo-1,2-dihydro-1,6-naphthyridine-3-carbonitrile

a) Preparation of 7-chloro-1-cyclopentyl-2-oxo-1,2-dihydro-1, 6-naphthyridine-3-carbonitrile: To solution of 6-chloro-4-(cyclopentylamino)nicotinaldehyde (430.0 mg, 2.0 mmol, 1.0 eq) and 2-cyanoacetic acid (250.0 mg, 2.8 mmol, 1.4 eq) in AcOH (10 mL) was added phenylmethanamine (20.0 mg, 0.2 mmol, 0.1 eq) at room temperature and the mixture was stirred for 6 h at 110° C. After completion of the reaction, the mixture was quenched with water (10 mL) and extracted with EA (10 mL×3). The combined organic phase was washed with aqueous solution of NaHCO₃ (20 mL), dried over anhydrous sodium sulfate and concentrated under reduced pressure. The crude product was triturated with i-PrOH (3 mL) to afford the product (280.0 mg, yield: 54%, white solid). LC-MS: 274.15 [M+1]⁺.
b) Preparation of 1-cyclopentyl-7-((2-methoxy-4-(4-methylpiperazin-1-yl)phenyl)amino)-2-oxo-1,2-dihydro-1,6-naphthyridine-3-carbonitrile: To a solution of 7-chloro-1-cyclopentyl -2-oxo-1,2-dihydro-1,6-naphthyridine-3-carbonitrile (120.0 mg, 0.4 mmol, 1.0 eq) in 2-BuOH (5 mL) was added TFA (20.0 mg, 0.2 mmol, 0.5 eq) at room temperature and the mixture was stirred at 120° C. overnight. After completion of the reaction, the mixture was concentrated under reduced pressure and the residue was purified by Prep-HPLC (C18, CH₃CN/H₂O, 15˜50%, 0.1% HCOOH added) to afford the target compound (34.0 mg, yield: 17%, slightly yellow solid).

The compounds of Examples 26-27 were prepared using a synthesis method similar to that described in Example 25.

| Example | R₀ | R₉ | R₁₀ | B₁ | MW | LC-MS | ¹H NMR (400 MHz) |
|---|---|---|---|---|---|---|---|
| 25 | 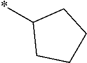 [SMILES: *C1CCCC1] | H | CN | CH | 458.56 | 459.15 [M + 1]⁺ | DMSO-d₆: δ 8.98 (s, 1H), 8.50 (s, 2H), 7.28 (d, J = 8.8 Hz, 1H), 6.67 (s, 1H), 6.53 (d, J = 8.8 Hz, 1H), 6.18 (s, 1H), 5.52-5.35 (m, 1H), |
| | | | | | | | 3.76 (s, 3H), 3.17 (t, J = 4.9 Hz, |
| | | | | | | | 4H), 2.47 (d, J = 5.5 Hz, 4H), 2.23 |
| | | | | | | | (s, 3H), 1.97-1.84 (m, 2H), 1.84- |
| | | | | | | | 1.71 (m, 2H), 1.64-1.45 (m, 4H). |
| 26 | 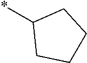 [SMILES: *C1CCCC1] | H | CN | N | 459.54 | 460.35 [M + 1]⁺, 458.35 [M − 1]⁻ | CDCl₃: δ 8.55 (s, 1H), 8.07 (d, J = 8.6 Hz, 1H), 7.94 (s, 1H), 6.55 (s, 2H), 5.97-5.77 (m, 1H), 3.91 (s, 3H), 3.32-3.20 (m, 4H), 2.78-2.63 |
| | | | | | | | (m, 4H), 2.44 (s, 3H), 2.37-2.23 |
| | | | | | | | (m, 2H), 2.12-1.96 (m, 2H), 1.92- |
| | | | | | | | 1.80 (m, 4H). |
| 27 | 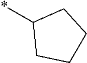 [SMILES: *C1CCCC1] | CH₃ | H | N | 448.57 | 449.20 [M + 1]⁺, 447.85 [M − 1]⁻ | DMSO-d₆: δ 8.71-8.66 (m, 1H), 8.65 (s, 1H), 7.28-7.22 (m, 1H), 6.59 (s, 1H), 6.50-6.43 (m, 1H), 6.09 (s, 1H), 5.63-5.45 (m, 1H), |
| | | | | | | | 3.72-3.67 (m, 3H), 3.14-3.09 (m, |
| | | | | | | | 4H), 2.45-2.41 (m, 4H), 2.36-2.25 |
| | | | | | | | (m, 3H), 2.23-2.16 (m, 3H), 2.15- |
| | | | | | | | 2.01 (m, 2H), 1.67-1.38 (m, 4H), |
| | | | | | | | 1.41-1.27 (m, 2H). |

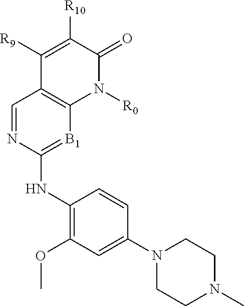

### Example 28

### 8-cyclopentyl-2-(2-methoxy-4-(4-methylpiperazin-1-yl)phenyl)amino)-5,8-dihydropyrido[2,3-d]pyrimidin-7(6H)-one

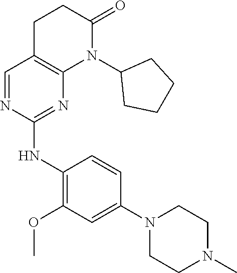 [SMILES: COc1cc(N2CCN(C)CC2)ccc1Nc1ncc2c(n1)N(C1CCCC1)C(=O)CC2] [0193] a) Preparation of 5-bromo-2-chloro-N-cyclopentylpyrimidin-4-amine: To solution of 5-bromo-2,4-dichloropyrimidine (10.0 g, 43.9 mmol, 1.0 eq) and triethylamine (8.9 g, 87.7 mmol, 2.0 eq) in dichloromethane (100 mL) was added cyclopentamine (4.1 g, 48.3 mmol, 1.1 eq) at 0° C. and the mixture was stirred at 0° C. for 30 min, then allowed to warm to room temperature and stirred overnight. After completion of the reaction, the mixture was quenched with water (100 mL) and extracted with DCM (100 mL×2). The combined organic phase was washed with brine, dried over anhydrous sodium sulfate and concentrated under reduced pressure to afford the product (8.0 g, yield: 66%, colorless oil). LC-MS: 275.90 [M+1]⁺.
b) Preparation of ethyl-3-(2-chloro-4-(cyclopentylamino)pyrimidin-5-yl)acrylate: To a solution of 5-bromo-2-chloro-N-cyclopentylpyrimidin-4-amine (1.0 g, 3.6 mmol, 1.0 eq) in THF (10 mL) was added ethyl acrylate (0.9 g, 9.1 mmol, 2.5 eq), (PhCN)₂PdCl₂ (140.0 mg, 0.4 mmol, 0.1 eq), P(o-tolyl)₃ (111.0 mg, 0.4 mmol, 0.1 eq), DIEA (2.3 g, 18.2 mmol, 5.0 eq). The mixture was stirred at 70° C. overnight under N₂ atmosphere. To the mixture was added water (20 mL) and the resulting mixture was extracted with DCM (50 mL×3). The organic phase was washed with brine, dried over anhydrous sodium sulfate and concentrated under reduced pressure to give the residue, which was purified by chromatography over silica gel (petroleumether: ethyl acetate=10:1) to afford the product (0.8 g, yield: 76%, yellow solid). LC-MS: 296.35 [M+1]⁺.
c) Preparation of ethyl-3-(4-(cyclopentylamino)-2-((2-methoxy-4-(4-methylpiperazin-1-yl)phenyl)amino)pyrimidin-5-yl)acrylate: To a solution of ethyl-3-(2-chloro-4-(cyclopentylamino)pyrimidin-5-yl)acrylate (810.0 mg, 2.7 mmol, 1.0 eq) in dioxane (20 mL) was added 2-methoxy-4-(4-methylpiperazin-1-yl)aniline (0.7 g, 3.3 mmol, 1.2 eq), Pd(OAc)₂ (123.0 mg, 0.55 mmol, 0.2 eq), BINAP (171.0 mg, 0.28 mmol, 0.1 eq), and Cs₂CO₃ (2.7 g, 8.25 mmol, 3.0 eq). The mixture was stirred at 90° C. for 2 h under N₂ atmosphere. To the mixture was added water (20 mL) and the resulting mixture was extracted with DCM (50 mL×3). The organic phase was washed with brine, dried over anhydrous sodium sulfate and the residue was purified by chromatography over silica gel (dichloromethane: MeOH=50:1) to afford the product (0.4 g, yield: 32%) as a yellow solid. LC-MS: 481.45 [M+1]⁺.
d) Preparation of ethyl 3-(4-(cyclopentylamino)-2-((2-methoxy-4-(4-methylpiperazin-1-yl)phenyl)amino)pyrimidin-5-yl)propanoate: A solution of ethyl-3-(4-(cyclopentylamino)-2-((2-methoxy-4-(4-methylpiperazin-1-yl)phenyl)amino)pyrimidin-5-yl)acrylate (0.4 g, 0.8 mmol, 1.0 eq) and 10% Pd/C (40.0 mg) in MeOH (10 mL) was stirred at rt under an hydrogen atmosphere overnight. After completion of the reaction, the mixture was filtered and the filtrate was concentrated. The residue was purified by chromatography over silica gel (dichloromethane:MeOH=50:1) to afford the product (230.0 mg, yield: 58%, light yellow solid). LC-MS: 483.25 [M+1]⁺.
e) Preparation of 3-(4-(cyclopentylamino)-2-((2-methoxy-4-(4-methylpiperazin-1-yl)phenyl)amino)pyrimidin-5-yl)propanoic acid: To a solution of ethyl 3-(4-(cyclopentylamino)-2-((2-methoxy-4-(4-methylpiperazin-1-yl)phenyl)amino)pyrimidin-5-yl)propanoate (0.7 g, 1.5 mmol, 1.0 eq) in MeOH (10 mL) was added 2 M NaOH aqueous solution (1.45 mL, 2.0 eq). The mixture was stirred at room temperature overnight. After completion of the reaction, to the mixture was added 6 M HCl aqueous solution to adjust pH=3, and then the aqueous phase was lyophilized to obtain the crude product used for the next step without further purification. LC-MS: 455.20 [M−1]⁺.
d) Preparation of 8-cyclopentyl-2-(2-methoxy-4-(4-methylpiperazin-1-yl)phenyl)amino)-5,8-dihydropyrido[2,3-d]pyrimidin-7(6H)-one: A solution of 3-(4-(cyclopentylamino)-2-((2-methoxy-4-(4-methylpiperazin-1-yl)phenyl)amino)pyrimidin-5-yl)propanoic acid (0.7 g, 1.5 mmol, 1.0 eq), HATU (1.1 g, 3.0 mmol, 2.0 eq), DIEA (0.5 g, 3.75 mmol, 2.5 eq) in DMF (10 mL) was stirred at room temperature overnight. After completion of the reaction, to the mixture was added H₂O (10 mL) and the mixture was extracted with EA (10 mL×3). The combined organic phase was washed with brine, dried over anhydrous sodium sulfate. The residue was purified by Prep-TLC (dichloromethane: methanol=10:1) and Prep-HPLC to afford the target compound (28.0 mg, yield: 4%, yellow solid).

The compounds of Examples 29-30 were prepared using a synthesis method similar to that described in Example 28.

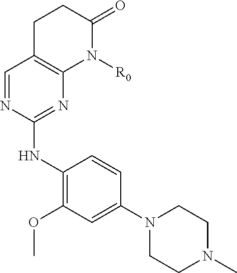

| Example | R₀ | MW | LC-MS | ¹H NMR (400 MHz) |
|---|---|---|---|---|
| 28 | 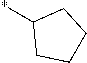 [SMILES: *C1CCCC1] | 436.56 | 437.50 [M + 1]⁺ | DMSO-d₆: δ 8.00 (s, 1H), 7.83 (s, 1H), 7.45 (d, J = 8.5 Hz, 1H), 6.57 (s, 1H), 6.42 (d, J = 8.4 Hz, 1H), 5.16-5.08 (m, 1H), 3.72 (s, 3H), 3.08 (d, J = 5.5 Hz, 4H), 2.61 (t, J = 7.2 Hz, 2H), 2.56- |
| | | | | 2.48 (m, 2H), 2.45-2.42 (m, 4H), 2.20 (s, 3H), |
| | | | | 2.04-1.93 (m, 2H), 1.67-1.52 (m, 4H), 1.39 (t, |
| | | | | J = 7.2 Hz, 2H). |
| 29 | 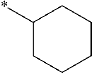 [SMILES: *C1CCCCC1] | 450.59 | 451.25 [M + 1]⁺ | CDCl₃: δ 8.16 (d, J = 8.5 Hz, 1H), 8.02 (s, 1H), 7.36 (s, 1H), 6.60-6.50 (m, 2H), 4.79 (t, J = 12.2 Hz, 1H), 3.88 (s, 3H), 3.24 (t, J = 5.0 Hz, 4H), 3.08 (q, J = 7.3 Hz, 2H), 2.77 (t, J = 4.9 Hz, 4H), 2.71 (t, J = 7.2 Hz, 2H), 2.62 (t, J = 7.4 Hz, 2H), |
| | | | | 2.50-2.43 (m, 5H), 1.85 (d, J = 12.7 Hz, 2H), |
| | | | | 1.66 (d, J = 11.1 Hz, 2H), 1.40-1.33 (m, 2H). |
| 30 | 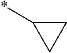 [SMILES: *C1CC1] | 408.51 | 409.20 [M + 1]⁺ | DMSO-d₆: δ 8.11 (d, J = 8.7 Hz, 1H), 8.05 (s, 1H), 7.69 (s, 1H), 6.68 (s, 1H), 6.53 (d, J = 8.7 Hz, 1H), 3.82 (s, 3H), 2.85-2.76 (m, 4H), 2.66- 2.59 (m, 2H), 2.59-2.51 (m, 3H), 2.50-2.46 (m, |
| | | | | 7H), 1.04-0.94 (m, 2H), 0.63-0.52 (m, 2H). |

### Example 31

### 8-cyclopentyl-2-((2-methoxy-4-(4-methylpiperazin-1-yl)phenyl)amino)-6H-pyrimido[5,4-b][1,4]oxazin-7(8H)-one

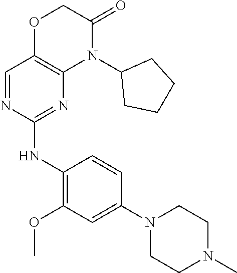 [SMILES: COc1cc(N2CCN(C)CC2)ccc1Nc1ncc2c(n1)N(C1CCCC1)C(=O)CO2] [0201] a) Preparation of 2-chloro-N-cyclopentyl-5-methoxypyrimidin-4-amine: To a solution of 2,4-dichloro-5-methoxypyrimidine (3.0 g, 16.9 mmol, 1.0 eq) and TEA (3.4 g, 33.8 mmol, 2.0 eq) in dichloromethane (30 mL) was added cyclopentamine (1.7 g, 20.2 mmol, 1.2 eq) at 0° C. The resulting solution was stirred at 0° C. for 30 min, and then warmed to room temperature and stirred overnight. After completion of the reaction, the reaction mixture was concentrated under reduced pressure to afford the product (3.5 g, yield: 93%, off white solid). LC-MS: 228.00 [M+1]⁺.
b) Preparation of 2-chloro-4-(cyclopentylamino)pyimidin-5-ol: To a solution of 2-chloro-N-cyclopentyl-5-methoxypyrimidin-4-amine (3.9 g, 17.2 mmol, 1.0 eq) in 1,2-dichloroethane (20 mL) was added BBr 3 (2 M in DCE, 34.4 mL, 68.8 mmol, 4.0 eq) dropwise at 0° C. The mixture was stirred at 0° C. for 40 min, and then heated to 80° C. and stirred for 1 hour. After completion of the reaction, the mixture was concentrated under reduced pressure. The residue was purified by chromatography over silica gel (petroleumether: ethyl acetate=2:1) to afford the product (2.9 g, yield: 80%, off white solid). LC-MS: 214.05 [M+1]⁺, 211.95 [M−1]⁻.
c) Preparation of 2-chloro-8-cyclopentyl-6H-pyimido[5,4-b][1,4]oxazin-7(8H)-one: To a solution of 2-chloro-4-(cyclopentylamino)pyrimidin-5-ol (220.0 mg, 1.04 mmol, 1.0 eq) in ACN (5 mL) was added ethyl bromoacetate (208.0 mg, 1.3 mmol, 1.2 eq) at 0° C. After stirred at 0° C. for 30 min, the solution was warmed to room temperature and stirred overnight. After completion of the reaction, the mixture was concentrated under reduced pressure. To the residue was added water (5 mL) and the solution was extracted with DCM (5 mL×3). The organic phase was combined and washed with brine, dried over anhydrous sodium sulfate and concentrated under reduced pressure to afford the product (222.0 mg, yield: 50%, yellow solid). LC-MS: 254.00[M+1]⁺.
d) Preparation of 8-cyclopentyl-2-((2-methoxy-4-(4-methylpiperazin-1-yl)phenyl)amino)-6H-pyrimido[5,4-b][1,4]oxazin-7(8H)-one: To a solution of 2-chloro-8-cyclopentyl-6H-pyrimido[5,4-b][1,4]oxazin-7(8H)-one (90.0 mg, 0.36 mmol, 1.0 eq) in dioxane (1.5 mL) was added Pd(OAc)₂(8.0 mg, 0.04 mmol, 0.1 eq), BINAP (44.3 mg, 0.2 eq, 0.07mmol), Cs₂CO₃(347.1 mg, 1.1 mmol, 3.0 eq) and 2-methoxy-4-(4-methylpiperazin-1-yl)aniline (78.7 mg, 0.36 mmol, 1.0 eq). The mixture was stirred at 100° C. for 1 h under N₂. After completion of the reaction, water (5 mL) was added and the mixture was extracted with EA (5 mL×3). The combined organic phase was washed with brine, dried over anhydrous sodium sulfate and concentrated under reduce pressure. The residue was triturated with EA/PE=3/1 (4 mL) to afford the target compound (50.0 mg, yield: 37%, yellow solid). LC-MS:439.40 [M+1]⁺. ¹H NMR (400 MHz, CDCl₃): δ 8.13 (d, J=8.8 Hz, 1H), 8.00-7.97 (m, 1H), 7.29 (s, 1H), 6.55 (s, 2H), 5.33-5.22 (m, 1H), 4.56-4.52 (m, 2H), 3.92-3.86 (m, 3H), 3.37 (s, 4H), 2.96 (s, 4H), 2.62 (s, 3H), 2.28-2.14 (m, 2H), 2.02-1.82 (m, 4H), 1.71-1.56 (m, 2H).

The compounds of Examples 32-33 were prepared using a synthesis method similar to that described in Example 2.

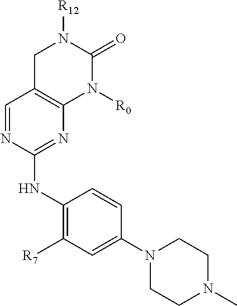

| Example | R₀ | R₇ | R₁₂ | MW | LC-MS | ¹H NMR (400 MHz) |
|---|---|---|---|---|---|---|
| 32 | 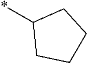 [SMILES: *C1CCCC1] | Br | H | 486.41 | 488.25 [M + 3]⁺ | CDCl₃: δ 8.02 (d, J = 8.9 Hz, 1H), 7.94 (s, 1H), 7.14 (s, 1H), 7.06 (s, 1H), 6.90 (d, J = 8.8 Hz, 1H), 5.25-5.05 (m, 1H), 4.29 (s, 2H), 3.35- 3.18 (m, 4H), 2.85-2.62 (m, 4H), 2.43 (s, 3H), |
| | | | | | | 2.30-2.12 (m, 2H), 1.91-1.76 (m, 4H), 1.62- |
| | | | | | | 1.50 (m, 2H). |
| 33 | 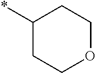 [SMILES: *C1CCOCC1] | OCH₃ | H | 453.54 | 454.40 [M + 1]⁺ | DMSO-d₆: δ 7.95 (s, 1H), 7.80 (s, 1H), 7.63 (d, J = 7.5 Hz, 1H), 7.32 (s, 1H), 6.59 (s, 1H), 6.44 (d, J = 7.5 Hz, 1H), 4.74-4.64 (m, 1H), 4.07 (s, 2H), 3.87 (d, J = 8.9 Hz, 2H), 3.77 (s, 3H), 3.26-3.20 (m, 2H), 3.08 (s, 4H), 2.64-2.54 |
| | | | | | | (m, 2H), 2.52-2.42 (m, 4H), 2.20 (s, 3H), 1.42 |
| | | | | | | (d, J = 10.7 Hz, 2H). |

### Example 34

### 8-cyclopentyl -N-(2-methoxy-4-(4-methylpiperazin-1-yl)phenyl)pyrido[4,3-d]pyrimidin-2-amine

a) Preparation of 8-iodo-2-(methylsulfinyl)pyrido[4,3-d]pyrimidine: To a solution of 8-iodo-2-(methylthio)pyrido[4,3-d]pyrimidine (350.0 mg, 1.2 mmol, 1.0 eq) in DCM (15 mL) was added 3-chloroperoxybenzoic acid (240.0 mg, 1.4 mmol, 1.2 eq) at −20° C. The mixture was stirred at −20° C. for 2 hours. After completion of the reaction, the mixture was quenched with aqueous sodium thiosulfate (10 mL) and the mixture was extracted with DCM (10 mL×3). The organic phase was separated, dried and concentrated to give the residue which was purified by chromatography over silica gel (MeOH: DCM=100:1˜10:1) to afford the product (200.0 mg, yield: 54%, yellow solid). LC-MS: 319.85 [M+1]⁺.
b) Preparation of 8-iodo-N-(2-methoxy-4-(4-methylpiperazin-1-yl)phenyl)pyrido[4,3-d]pyrimidin-2-amine: To a solution of 8-iodo-2-(methylsulfinyl)pyrido[4,3-d]pyrimidine (200.0 mg, 0.6 mmol) and 2-methoxy-4-(4-methylpiperazin-1-yl)aniline (166.0 mg, 0.7 mmol) in ACN (5 mL) was added TFA (358.0 mg, 3.1 mmol). The mixture was refluxed at 85° C. for 12 hours. After completion of the reaction, water (10 mL) was added and the mixture was extracted with EA (10 mL×3). The organic phase was washed with brine, dried over anhydrous sodium sulfate and concentrated under reduced pressure. The residue was purified by chromatography over silica gel (DCM:MeOH=100:1˜20:1) to afford the product (160.0 mg, 2 steps yield: 29%, red solid). LC-MS: 477.05 [M+1]⁺.
c) Preparation of 8-(cyclopent-1-en-1-yl)-N-(2-methoxy-4-(4-methylpiperazin-1-yl)phenyl)pyrido[4,3-d]pyrimidin-2-amine: To a solution of 8-iodo-N-(2-methoxy-4-(4-methylpiperazin-1-yl)phenyl)pyrido[4,3-d]pyrimidin-2-amine (160.0 mg, 0.3 mmol) in dioxane (3 mL) and water (3 mL) was added 2-(cyclopent-1-en-1-yl)-4,4,5,5-tetramethyl-1,3,2-dioxaborolane (98.0 mg, 0.5 mmol), NaHCO₃ (56.0 mg, 0.7 mmol) and Pd(PPh₃)₂Cl₂ (69.0 mg, 0.1 mmol). The system was evacuated and backfilled with nitrogen three times and the mixture was stirred at 130° C. for 0.5 hour. After completion of the reaction, water (10 mL) was added and the mixture was extracted with EA (10 mL×3). The organic phase was washed with brine, dried over anhydrous sodium sulfate and concentrated under reduce pressure. The residue was purified by Prep-TLC (DCM:MeOH=10:1) to afford the product (65.0 mg, yield: 55%, yellow solid). LC-MS: 417.15 [M+1]⁺.
d) Preparation of 8-cyclopentyl-2-(2-methoxy-4-(4-methylpip erazin-1-yl)phenyl)amino)pteridin-7(8H)-one: To a solution of 8-(cyclopent-1-en-1-yl)-N-(2-methoxy-4-(4-methylpiperazin-1-yl)phenyl)pyrido[4,3-d]pyrimidin-2-amine (65.0 mg, 0.2 mmol) and TosNHNH₂ (290.0 mg, 1.6 mmol) in THF (10 mL) and water (2 mL) was added NaOAc (128.0 mg, 1.6 mmol). The mixture was refluxed at 85° C. for 48 hours. After completion of the reaction, water (10 mL) was added and the mixture was extracted with EA (10 mL×3). The combined organic phase was washed with brine, dried over anhydrous sodium sulfate and concentrated under reduce pressure. The residue was purified by Prep-HPLC (C18, CH₃CN/H₂O, 10˜40%, 0.1% HCOOH added) to afford the target compound (22.0 mg, yield: 33%, yellow solid).

The compound of Example 35 was prepared using a synthesis method similar to that described in Example 34.

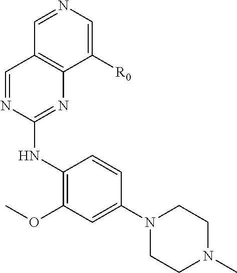

| Example | R₀ | MW | LC-MS | ¹H NMR (400 MHz) |
|---|---|---|---|---|
| 34 | 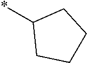 [SMILES: *C1CCCC1] | 418.55 | 419.40 [M + 1]⁺ | DMSO-d₆: δ 9.36-9.29 (m, 1H), 8.94 (s, 1H), 8.66 (s, 1H), 8.50 (s, 1H), 8.30 (s, 1H), 6.70- 6.63 (m, 1H), 6.57-6.48 (m, 1H), 3.81 (s, 3H), 3.19-3.11 (m, 4H), 2.49-2.43 (m, 5H), 2.23 (s, |
| | | | | 3H), 2.06-1.95 (m, 2H), 1.85-1.71 (m, 4H), |
| | | | | 1.69-1.59 (m, 2H). |
| 35 | 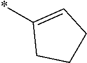 [SMILES: *C1=CCCC1] | 416.52 | 417.15 [M + 1]⁺ | CDCl₃: δ 9.10 (s, 1H), 8.82 (s, 1H), 8.58 (s, 1H), 8.50 (brs, 1H), 7.88 (s, 1H), 7.06 (s, 1H), 6.60-6.54 (m, 2H), 3.90 (s, 3H), 3.24 (t, J = 4.9 Hz, 4H), 2.96-2.86 (m, 2H), 2.71-2.59 (m, |
| | | | | 6H), 2.40 (s, 3H), 2.07 (p, J = 7.5 Hz, 2H). |

### Example 36

### 6-acetyl-8-cyclopentyl ethoxy-4-(4-methyl piperazi n-1-yl)phenyl)ami no)-5-methylpyri do [2,3-d]pyrimidin-7(8H)-one

a) Preparation of 6-b rom o-2-chloro-8-cyclopentyl -5-methylpyrido[2,3-d]pyrimidin-7(8H)-one: To a solution of 2-chloro-8-cyclopentyl-5-methylpyrido[2,3-d]pyrimidin-7(8H)-one (500.0 mg, 1.9 mmol) in ACN (10 mL) was added oxalic acid (35.0 mg, 0.38 mmol), acetic anhydride (250.0 mg, 50% W/W), and NBS (405.0 mg, 2.3 mmol). The mixture was stirred at 60° C. overnight under N2 atmosphere. Solution of sodium hyposulfite (10 mL) was added, the mixture was filtered and the filtrate was concentrated under reduced pressure to afford the product (410.0 mg, yield: 64%, white solid). LC-MS: 342.15 [M+1]⁺.
b) Preparation of 6-acetyl-2-chloro-8-cyclopentyl-5-methylpyrido[2,3-d]pyrimidin-7(8H)-one: To a solution of 2-chloro-8-cyclopentyl-5-methylpyrido[2,3-d]pyrimidin-7(8H)-one (200.0 mg, 0.6 mmol) in NMP (5 mL) was added tributyl(1-ethoxyvinyl)stannane (264.7 mg, 0.7 mmol) and bis(tri-t-butylphosphine)palladium (14.9 mg, 0.02 mmol). The mixture was stirred at room temperature overnight under N₂ atmosphere. After completion of the reaction, 1 N HCl (3.5 mL) was added and the mixture was stirred for 30 min. Then saturated solution of Na₂CO₃ was added to adjust pH>7. The mixture was extracted with EA (20 mL×3). The organic phase was washed with brine, dried over anhydrous sodium sulfate and concentrated. The residue was purified by chromatography over silica gel (petroleumether: ethyl acetate =4:1) to afford the product (93.3 mg, yield: 36%, off white solid). LC-MS: 306.05 [M+1]⁺.
c) Preparation of 6-acetyl-8-cyclopentyl-2-((2-methoxy-4-(4-methylpiperazin-1-yl)phenyl)amino)-5-methylpyrido[2,3-d]pyrimidin-7(8H)-one: To a solution of 6-acetyl-2-chloro-8-cyclopentyl-5-methylpyrido[2,3-d]pyrimidin-7(8H)-one (200.0 mg, 0.6 mmol) in dioxane (5 mL) was added 2-methoxy-4-(4-methylpiperazin-1-yl)aniline (145.1 mg, 0.6 mmol), Pd-G3 (110.9 mg, 0.1 mmol) and Cs₂CO₃ (481.7 mg, 1.5 mmol). The mixture was stirred at 100° C. overnight under N₂ atmosphere. To the mixture was added water (20 mL) and the mixture was extracted with DCM (20 mL×3). The organic phase was washed with brine, dried over anhydrous sodium sulfate and concentrated. The residue was purified by Prep-TLC (DCM:MeOH=10:1) and Prep-HPLC (C18, CH₃ CN/H₂O, 10˜40%, 0.1% HCOOH) to afford the target compound (11.2 mg, yield: 4%, brown solid).

The compound of Example 37 was prepared using a synthesis method similar to that described in Example 36.

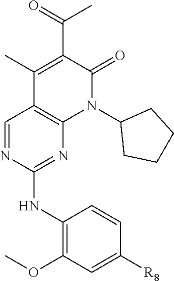

| Example | R₈ | MW | LC-MS | ¹H NMR (400 MHz) |
|---|---|---|---|---|
| 36 | 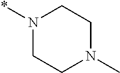 [SMILES: *N1CCN(C)CC1] | 490.61 | 491.20 [M + 1]⁺ | DMSO-d₆: δ 8.92 (s, 1H), 8.83 (s, 1H), 7.28 (d, J = 7.9 Hz, 1H), 6.64 (d, J = 2.0 Hz, 1H), 6.51 (dd, J = 9.0, 1.8 Hz, 1H), 5.69-5.45 (m, 1H), 3.74 (s, 3H), 3.20-3.17 (m, 4H), 2.60-2.53 (m, 4H), 2.40 (s, 3H), 2.30 (s, 3H), 2.27 (s, 3H), |
| | | | | 2.14 (s, 2H), 1.72-1.50 (m, 4H), 1.40 (s, 2H). |
| 37 | 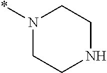 [SMILES: *N1CCNCC1] | 476.58 | 477.20 [M + 1]⁺ | DMSO-d₆: δ 8.94 (s, 1H), 8.83 (s, 1H), 8.29 (s, 1H), 7.28 (d, J = 8.7 Hz, 1H), 6.63 (d, J = 2.0 Hz, 1H), 6.50 (dd, J = 8.6, 2.0 Hz, 1H), 5.58 (brs, 1H), 3.74 (s, 3H), 3.13-3.08 (m, 4H), 2.95- 2.89 (m, 4H), 2.40 (s, 3H), 2.27 (s, 3H), 2.14 |
| | | | | (s, 2H), 1.65-1.45 (m, 4H), 1.45-1.30 (m, 2H). |

### Example 38-40

The compounds of Examples 38-40 were prepared using a synthesis method similar to that described in Example 19.

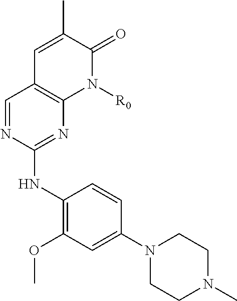

| Example | R₀ | MW | LC-MS | ¹H NMR (400 MHz) |
|---|---|---|---|---|
| 38 | 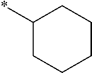 [SMILES: *C1CCCCC1] | 462.60 | 463.20 [M + 1]⁺ | CDCl₃: δ 8.40 (s, 1H), 8.30-8.17 (m, 1H), 7.73-7.54 (m, 1H), 7.30 (s, 1H), 6.64-6.49 (m, 2H), 5.58-5.22 (m, 1H), 3.88 (s, 3H), 3.27-3.22 (m, 4H), 2.80-2.65 (m, 4H), 2.44 (s, 3H), 2.14 (s, 3H), 2.05-1.95 (m, 2H), 1.92-1.86 (m, 2H), |
| | | | | 1.79-1.58 (m, 2H), 1.50-1.35 (m, 2H), 1.34- |
| | | | | 1.15 (m, 2H). |
| 39 | 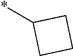 [SMILES: *C1CCC1] | 434.54 | 435.25 [M + 1]⁺ | CDCl₃: δ 8.40 (s, 1H), 8.19 (d, J = 8.5 Hz, 1H), 7.52 (s, 1H), 7.29 (s, 1H), 6.71-6.40 (m, 2H), 6.11-5.66 (m, 1H), 3.88 (s, 3H), 3.32-3.00 (m, 6H), 2.71-2.58 (m, 4H), 2.37 (s, 3H), 2.32-2.22 |
| | | | | (m, 2H), 2.14 (s, 3H), 2.04-1.92 (m, 1H), 1.89- |
| | | | | 1.73 (m, 1H). |
| 40 | 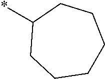 [SMILES: *C1CCCCCC1] | 476.63 | — | — |

### Example 41

### 8-cycloheptyl-2-(2-methoxy-4-(4-methylpiperazin-1-yl)phenyl)amino)pyrido[2,3-d]pyrimidin-7(8H)-one

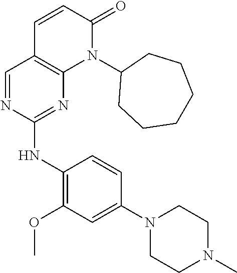 [SMILES: COc1cc(N2CCN(C)CC2)ccc1Nc1ncc2ccc(=O)n(C3CCCCCC3)c2n1]

The compound of this embodiment was prepared using a synthesis method similar to that described in Example 19.

### Example 42

### In Vitro Assay to Determine the Inhibitory Effect of Compounds in the Patent on NUAK1 Enzymatic Activity

The in vitro NUAK1 enzymatic assay was carried out at Eurofins Discovery to evaluate the inhibitory effect of the compounds presented in the invention. The assay protocol is as follow: the compound to be tested was mixed with NUAK1(h) enzyme and 300 μM KKKVSRSGLYRSPSMPENLNRPR substrate in 8 mM MOPS (pH 7.0) buffer containing 0.2 mM EDTA and incubated at room temperature. A Mg/ATP mixture containing 10 mM magnesium acetate and 45 μM [y-33P]-ATP was added to start the reaction. After incubation for 40 min at room temperature, the reaction was terminated by adding 0.5% phosphoric acid. Then, 10 μL of the reaction mix was spotted onto a P30 filter paper followed with four times of wash with 0.425% phosphoric acid solution (4 min each time), and one time wash with methanol. The filter paper was air-dried and counted in a liquid scintillation counter. The values obtained were plotted against the drug concentration and the IC₅₀ was calculated.

Table 1 summarizes the inhibitory effect (IC₅₀) of compounds tested on the NUAK1 enzyme activity in the present invention. ++++ indicates IC₅₀≤10 nM; ++ indicates 10 nM<IC₅₀≤100 nM; ++ indicates 100 nM<IC₅₀≤1 μM; + indicates IC₅₀>1 μM.

TABLE 1

| Example | 1 | 2 | 3 | 4 | 5 | 6 |
|---|---|---|---|---|---|---|
| IC₅₀ | + | +++ | ++ | ++ | ++ | ++ |

| Example | 7 | 8 | 9 | 10 | 11 | 12 |
|---|---|---|---|---|---|---|
| IC₅₀ | + | + | + | ++ | ++ | ++ |

| Example | 13 | 14 | 15 | 16 | 19 | 20 |
|---|---|---|---|---|---|---|
| IC₅₀ | + | ++ | ++ | + | +++ | +++ |

| Example | 21 | 22 | 23 | 24 | 25 | 26 |
|---|---|---|---|---|---|---|
| IC₅₀ | +++ | ++++ | +++ | ++ | + | +++ |

| Example | 27 | 28 | 29 | 30 | 31 | 32 |
|---|---|---|---|---|---|---|
| IC₅₀ | ++ | ++ | +++ | ++ | ++ | +++ |

| Example | 33 | 34 | 35 | 36 | 37 | HTH-02-006 |
|---|---|---|---|---|---|---|
| IC₅₀ | ++ | +++ | +++ | + | + | +++ |

Accordingly, the compounds presented in the invention showed good inhibitory effect on NUAK1 enzyme.

### Example 43

### In Vitro Assay to Determine the Inhibitory Effect of Compounds in the Patent on NUAK2 Enzymatic Activity

The in vitro NUAK2 enzymatic assay was carried out at Eurofins Discovery to evaluate the inhibitory effect of the compounds presented in the invention. The assay protocol is as follow: the compound to be tested was mixed with NUAK2(h) enzyme and 300 KKKVSRSGLYRSPSMPENLNRPR substrate in 8 mM MOPS (pH 7.0) buffer containing 0.2 mM EDTA. A Mg/ATP mixture containing 10 mM magnesium acetate and 45 μM [γ-33P]-ATP was added to start the reaction. After incubation for 40 min at room temperature, the reaction was terminated by adding 0.5% phosphoric acid. Then 10 μL of the reaction mix was spotted onto a P30 filter paper. The filter paper was washed four times with 0.425% phosphoric acid with 4 min for each wash, and one time with methanol. The filter paper was air-dried and counted in a liquid scintillation counter. The values obtained were plotted against the drug concentration and the IC₅₀ was calculated.

Table 1 summarizes the inhibitory effect (IC₅₀) of compounds on the NUAK2 enzyme activity in the present invention. ++++ indicates IC₅₀≤10 nM; +++ indicates 10 nM<IC₅₀≤100 nM; ++ indicates 100 nM<IC₅₀≤1 μM; + indicates IC₅₀>1 μM.

TABLE 2

| Example | 1 | 2 | 3 | 4 | 5 | 6 |
|---|---|---|---|---|---|---|
| IC₅₀ | ++ | +++ | + | ++ | ++++ | +++ |

| Example | 7 | 8 | 9 | 10 | 11 | 12 |
|---|---|---|---|---|---|---|
| IC₅₀ | ++ | + | + | +++ | ++ | ++ |

| Example | 13 | 14 | 15 | 16 | 19 | 20 |
|---|---|---|---|---|---|---|
| IC₅₀ | + | ++ | + | ++ | ++++ | +++ |

| Example | 21 | 22 | 23 | 24 | 25 | 26 |
|---|---|---|---|---|---|---|
| IC₅₀ | ++ | ++++ | ++ | +++ | ++ | +++ |

| Example | 27 | 28 | 29 | 30 | 31 | 32 |
|---|---|---|---|---|---|---|
| IC₅₀ | ++ | +++ | +++ | + | ++ | +++ |

| Example | 33 | 34 | 35 | 36 | 37 | HTH-02-006 |
|---|---|---|---|---|---|---|
| IC₅₀ | ++ | +++ | +++ | ++ | ++ | ++ |

Accordingly, the compounds presented in the current invention showed good inhibitory effect on NUAK2 enzyme.

### Example 44

### CTG Assay to Evaluate the Inhibitory Effect of Compounds on HEC-1-B Human Endometrial Cancer Cells

Human endometrial cancer HEC-1-B cells were resuscitated and cultured in complete medium (MEM medium+10% FBS). When reaching about 80% confluence, the cells were gently blown away from the bottom of the culture dish with a 1 mL pipette. The cells were collected and centrifuged at 500 rpm for 3 min. The supernatant was discarded and the cell pellet was resuspend in fresh medium. Cells were counted to determine cell density and seeded into 96-well plates at a density of 1000 cells per well and incubated overnight at 37° C. in a 5% CO2 incubator. In the next day, the compounds in storage solution was serially diluted with DMSO at a ratio of 1:3 for a total of 9 concentrations. Two μL of each serially diluted solution was added to 98 μL of cell culture medium (10-fold dilution), while DMSO was mixed with culture medium similarly to be served as control. The compound dilutions were shaken and mixed. The cell culture plate with cells was removed from CO₂ incubator, the culture medium was aspirated from each well, 135 μL of fresh medium was added to each well followed with 15 μL of the compound dilutions. The plates were incubated at 37° C. for 7 days in a 5% CO₂ incubator. One hundred μL CellTiter-Glo reagent was added to each well and the plate was vortexed for 2 min and incubated for 10 min to fully lyse the cells. The chemiluminescence values were read using a plate reader.

The chemiluminescence values were plotted against compound concentrations to calculate the inhibitory activity of each compound on cell proliferation Cell inhibition rate (%)=(chemiluminescence value _{tested compound}−chemiluminescence value _{DMSO control})/(chemiluminescence value _{medium control}−chemiluminescence value_{DMSO control})×100%.

A nonlinear S-curve regression was fitted using XLFit software to fit the data to derive a dose-effect curve, from which the IC₅₀ values were calculated. The curve equation was Y=Bottom+(Top-Bottom)/(1+10\acute{ }((LogIC₅₀-X)×slope)), where Y is the cellular inhibition rate, X is the compound concentration, Bottom refers to the lowest inhibition rate, and Top refers to the highest inhibition rate.

Table 3 summarizes the inhibitory effects (IC₅₀) of the compounds on the proliferation of Hec-1B cells, Where ++++ indicates 100 nM<IC₅₀≤500 nM; +++ indicates 500 nM<IC₅₀≤1 μM; ++ indicates 1 μM<IC₅₀≤10 μM; + indicates IC₅₀>10 μM.

TABLE 3

| Example | 2 | 5 | 6 | 10 | 19 | 20 |
|---|---|---|---|---|---|---|
| IC₅₀ | ++ | +++ | +++ | +++ | +++ | + |

| Example | 21 | 22 | 23 | 24 | 26 | 27 |
|---|---|---|---|---|---|---|
| IC₅₀ | ++++ | + | ++++ | +++ | +++ | ++ |

| Example | 28 | 29 | 32 |
|---|---|---|---|
| IC₅₀ | ++ | +++ | ++ |

Therefore, the compound has good inhibitory effect on the growth of human endometrial cancer cells HEC-1-B by CTG detection method.

Having now fully described this disclosure, it will be understood by those of ordinary skill in the art that the same can be performed within a wide and equivalent range of conditions, formulations and other parameters without affecting the scope of the disclosure or any embodiment thereof. All patents, patent applications and publications cited herein are fully incorporated by reference herein in their entirety.

1. A compound of Formula I: 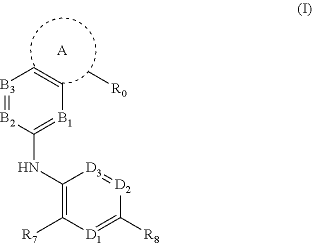
or pharmaceutically acceptable salts thereof, or geometric isomers, enantiomers, diastereomers, racemates, isotope-substituted derivatives, solvates, hydrates or prodrugs thereof, wherein:
A ring is an optionally substituted heterocyclic group or an optionally substituted heteroaryl;
R₀ is selected from an optionally substituted carbocyclic group and an optionally substituted heterocyclic group;
B₁ is selected from N and CR₁; B₂ is selected from N and CR₂; B₃ is selected from N and CR₃;
D₁ is selected from N and CR₄; D₂ is selected from N and CR₅; D₃ is selected from N and CR₆;
R₁, R₂, R₃, R₄, R₅, and R₆ are each independently selected from H, halogen, an optionally substituted alkyl, and an optionally substituted alkoxy;
R₇ is selected from halogen, an optionally substituted C₁-C₆ alkyl and an optionally substituted C₁-C₆ alkoxy;
R₈ is selected from an optionally substituted alkyl, an optionally substituted alkoxy, an optionally substituted amino, an optionally substituted heterocyclic group and an optionally substituted heteroaryl; or R₈ is connected to D₁ or to D₂ to form an optionally substituted heterocyclic group or an optionally substituted heteroaryl.

2. The compound of claim 1, or pharmaceutically acceptable salts thereof, or geometric isomers, enantiomers, diastereomers, racemates, isotope-substituted derivatives, solvates, hydrates or prodrugs thereof, wherein the compound of Formula I is represented by Formula Ia or Ib:

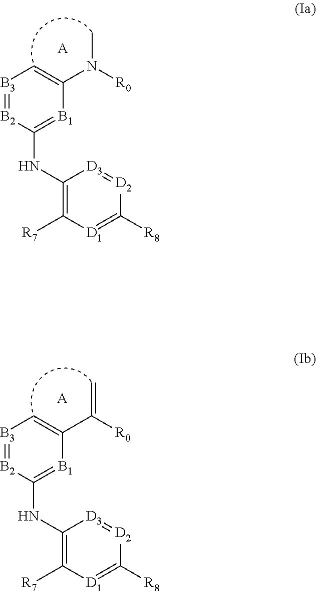

3. The compound of claim 1, or pharmaceutically acceptable salts thereof, or geometric isomers, enantiomers, diastereomers, racemates, isotope-substituted derivatives, solvates, hydrates or prodrugs thereof, wherein
R₀ is optionally substituted C₃-C₇ cycloalkyl, optionally substituted C₃-C₇ cycloalkenyl or optionally substituted 3-7 membered heterocyclic group;
(2) B₁ is N or CR₁, B₂ is N, B₃ is N or CR₃, R₁ is H, halogen or C₁-C₄ alkyl, R₃ is H, halogen or C₁-C₄ alkyl; or B₁ is N, B₂ is N, B₃ is CR₃, wherein R₃ is H or C₁-C₄ alkyl; or B₁ is N, B₂ is N, and B₃ is CH;
(3) D₁, D₂ and D₃ are each independently N or CH; or D₁ is CR₄, D₂ is CR₅, and D₃ is CR₆; R₄, R₅ and R₆ are each independently H, halogen and C₁-C₄ alkyl; or all of D₁, D₂ and D₃ are CH;
(4) R₇ is selected from halogen, optionally substituted C₁-C₃ alkyl and optionally substituted C₁-C₃ alkoxy; and
(5) R₈ is selected from optionally substituted C₁-C₆ alkyl, optionally substituted C₁-C₆ alkoxy, optionally substituted C₁-C₆ alkylamino and optionally substituted 4-7 membered nitrogen or oxygen-containing heterocyclic group.

4. The compound of claim 1, or pharmaceutically acceptable salts thereof, or geometric isomers, enantiomers, diastereomers, racemates, isotope-substituted derivatives, solvates, hydrates or prodrugs thereof, wherein the compound of Formula I is represented by Formula IIa, IIb or IIc: 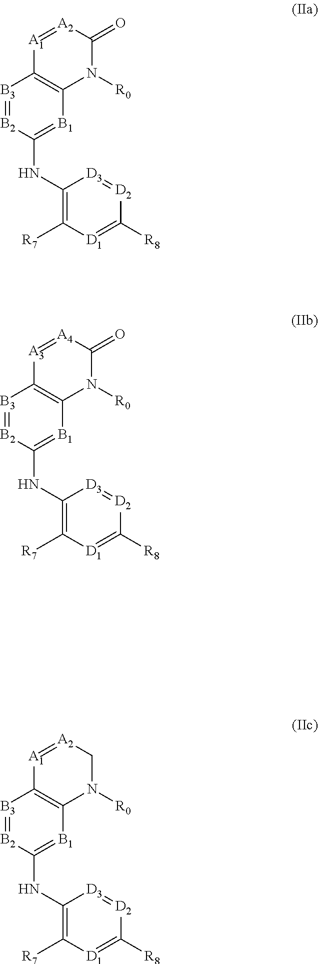
wherein;
A₁ is selected from N and CR₉;
A₂ is selected from N and CR₁₀;
A₃ is selected from O, S, NR₁₁ and CR₁₃R′₁₃;
A₄ is selected from O, S, NR₁₂ and CR₁₄R′₁₄;
R₉ and R₁₀ are each independently H, halogen, cyano, optionally substituted C₁-C₆ alkyl, optionally substituted C₁-C₆ alkoxy or optionally substituted C₁-C₆ acyl;
R₁₁ and R₁₂ are each independently H or optionally substituted C₁-C₆ alkyl;
R₁₃, R′₁₃, R₁₄ and R′₁₄ are each independently H, optionally substituted C₁-C₆ alkyl or optionally substituted C₁-C₆ alkoxy.

5. The compound claim 1, or pharmaceutically acceptable salts thereof, or geometric isomers, enantiomers, diastereomers, racemates, isotope-substituted derivatives, solvates, hydrates or prodrugs thereof, wherein, the fused bicyclic ring formed by ring A and the ring containing B₁-B₃ is selected from: 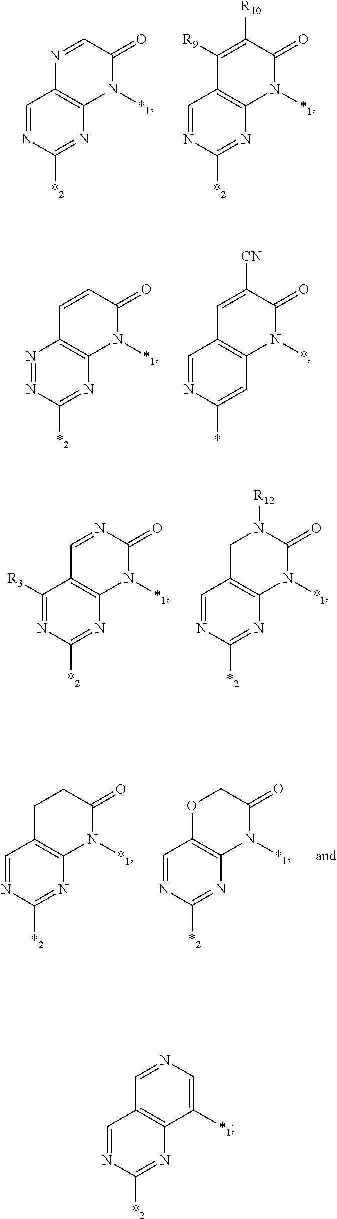
wherein, *₁ and *₂ refer to the attachment positions of the said groups to R₀ and —NH in the rest of the compound; R₃ is H or C₁-C₃ alkyl; R₉ is H or C₁-C₃ alkyl; R₁₀ is H, C₁-C₃ alkyl, C₂-C₄ acyl or cyano; R₁₂ is H or C₁-C₃ alkyl.

6. The compound of claim 1, or pharmaceutically acceptable salts thereof, or geometric isomers, enantiomers, diastereomers, racemates, isotope-substituted derivatives, solvates, hydrates or prodrugs thereof, wherein the compound of Formula I is represented by any one of Formulae (IIIa), (IIIb), (IIIc), (IIId), or (IIIe): 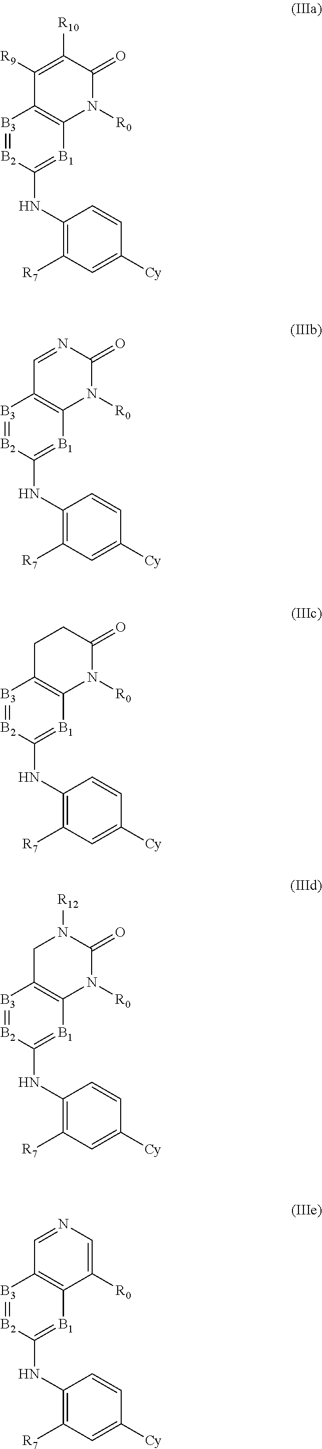
wherein R₉ and R₁₀ are each independently H, halogen, cyano, optionally substituted C₁-C₆ alkyl, optionally substituted C₁-C₆ alkoxy or optionally substituted C₁-C₆ acyl; R₁₂ is H or optionally substituted C₁-C₆ alkyl; and Cy is a 4-7 membered heterocyclic group optionally substituted by 1, 2 or 3 groups selected from the group consisting of C₁-C₆ alkyl, C₁-C₆ alkyl substituted with hydroxy, halogenated C₁-C₆ alkyl, C₁-C₆ alkoxy, C₁-C₆ acyl, heterocyclic group optionally substituted with 1-4 C₁-C₆ alkyl, halogen, —NRₐR_{b} and hydroxyl, wherein Rₐ and R_{b} are each independently H or C₁-C₆ alkyl.

7. The compound of claim 1, or pharmaceutically acceptable salts thereof, or geometric isomers, enantiomers, diastereomers, racemates, isotope-substituted derivatives, solvates, hydrates or prodrugs thereof, wherein the compound is selected from the group consisting of:
8-cyclopentyl-2-((2-methoxy-4-(4-methylpiperazin-1-yl)phenyl)amino)pteridin-7(8H)-one;
1-cyclopentyl-7-((2-methoxy-4-(4-methylpiperazin-1-yl)phenyl)amino)-3,4-dihydropyrimido[4,5-d]pyrimidin-2(1H)-one;
1-cyclopropyl-7-((2-methoxy-4-(4-methylpiperazin-1-yl)phenyl)amino)-3,4-dihydropyrimido[4,5-d]pyrimidin-2(1H)-one;
1-cyclopentyl-7-((2-methoxy-4-(4-methylpiperazin-1-yl)phenyl)amino)-3-methyl-3,4-dihydropyrimido[4,5-d]pyrimidin-2(1H)-one;
1-cyclopentyl-7-((2-methoxy-4-(4-methylpiperazin-1-yl)phenyl)amino)pyrimido[4,5-d]pyrimidin-2(1H)-one;
1-cyclohexyl-7-((2-methoxy-4-(4-methylpiperazin-1-yl)phenyl)amino)pyrimido[4,5-d]pyrimidin-2(1H)-one;
1-cyclopentyl-7-((2-ethoxy-4-(4-methylpiperazin-1-yl)phenyl)amino)pyrimido[4,5-d]pyrimidin-2(1H)-one;
1-cyclopentyl-7-((2-isopropoxy-4-(4-methylpiperazin-1-yl)phenyl)amino)pyrimido[4,5-d]pyrimidin-2(1H)-one;
1-cyclopentyl-7-((4-(4-methylpiperazin-1-yl)-2-(trifluoromethoxy)phenyl)amino)pyrimido[4,5-d]pyrimidin-2(1H)-one;
1-cyclopentyl-7-((2-methyl-4-(4-methylpiperazin-1-yl)phenyl)amino)pyrimido[4,5-d]pyrimidin-2(1H)-one;
7-((2-chloro-4-(4-methylpiperazin-1-yl)phenyl)amino)-1-cyclopentylpyrimido[4,5-d]pyrimidin-2(1H)-one;
7-((2-bromo-4-(4-methylpiperazin-1-yl)phenyl)amino)-1-cyclopentylpyrimido[4,5-d]pyrimidin-2(1H)-one;
1-cyclopentyl-7-((2-methoxy-4-(4-methylpiperazin-1-yl)phenyl)amino)-5-methylpyrimido[4,5-d]pyrimidin-2(1H)-one;
1-cyclobutyl-7-((2-methoxy-4-(4-methylpiperazin-1-yl)phenyl)amino)pyrimido[4,5-d]pyrimidin-2(1H)-one;
1-cyclopropyl-7-((2-methoxy-4-(4-methylpiperazin-1-yl)phenyl)amino)pyrimido[4,5-d]pyrimidin-2(1H)-one;
7-((2-methoxy-4-(4-methylpiperazin-1-yl)phenyl)amino)-1-(tetrahydro-2H-pyran-4-yl)pyrimido[4,5-d]pyrimidin-2(1H)-one;
7-((2-methoxy-4-(4-methylpiperazin-1-yl)phenyl)amino)-1-(piperidin-4-yl)pyrimido[4,5-d]pyrimidin-2(1H)-one;
7-((2-methoxy-4-(4-methylpiperazin-1-yl)phenyl)amino)-1-(1-methylpiperidin-4-yl)pyrimido[4,5-d]pyrimidin-2(1H)-one;
8-cyclopentyl-2-((2-methoxy-4-(4-methylpiperazin-1-yl)phenyl)amino)pyrido[2,3-d]pyrimidin-7(8H)-one;
8-cyclopentyl-2-((2-methoxy-4-(4-methylpiperazin-1-yl)phenyl)amino)-6-methylpyrido[2,3-d]pyrimidin-7(8H)-one;
8-cyclopropyl-2-(2-methoxy-4-(4-methylpiperazin-1-yl)phenyl)amino)-6-methylpyrido[2,3-d]pyrimidin-7(8H)-one;
8-cyclohexyl-2-((2-methoxy-4-(4-methylpiperazin-1-yl)phenyl)amino)pyrido[2,3-d]pyrimidin-7(8H)-one;
8-cyclopropyl-2-(2-methoxy-4-(4-methylpiperazin-1-yl)phenyl)amino)pyrido[2,3-d]pyrimidin-7(8H)-one;
5-cyclopentyl-3-((2-methoxy-4-(4-methylpiperazin-1-yl)phenyl)amino)pyrido[2,3-e][1,2,4]triazin-6(5H)-one;
1-cyclopentyl-7-((2-methoxy-4-(4-methylpiperazin-1-yl)phenyl)amino)-2-oxo-1,2-dihydro-1,6-naphthyridine-3-carbonitrile;
8-cyclopentyl-2-((2-methoxy-4-(4-methylpiperazin-1-yl)phenyl)amino)-7-oxo-7,8-dihydropyrido[2,3-d]pyrimidine-6-carbonitrile;
8-cyclopentyl-2-((2-methoxy-4-(4-methylpiperazin-1-yl)phenyl)amino)-5-methylpyrido[2,3-d]pyrimidin-7(8H)-one;
8-cyclopentyl-2-((2-methoxy-4-(4-methylpiperazin-1-yl)phenyl)amino)-5,8-dihydropyrido[2,3-d]pyrimidin-7(6H)-one;
8-cyclohexyl-2-((2-methoxy-4-(4-methylpiperazin-1-yl)phenyl)amino)-5,8-dihydropyrido[2,3-d]pyrimidin-7(6H)-one;
8-cyclopropyl-2-(2-methoxy-4-(4-methylpiperazin-1-yl)phenyl)amino)-5,8-dihydropyrido[2,3-d]pyrimidin-7(6H)-one;
8-cyclopentyl-2-((2-methoxy-4-(4-methylpiperazin-1-yl)phenyl)amino)-6H-pyrimido[5,4-b][1,4]oxazin-7(8H)-one;
7-((2-bromo-4-(4-methylpiperazin-1-yl)phenyl)amino)-1-cyclopentyl-3,4-dihydropyrimido[4,5-d]pyrimidin-2(1H)-one;
7-((2-methoxy-4-(4-methylpiperazin-1-yl)phenyl)amino)-1-(tetrahydro-2H-pyran-4-yl)-3,4-dihydropyrimido[4,5-d]pyrimidin-2(1H)-one;
8-cyclopentyl-N-(2-methoxy-4-(4-methylpiperazin-1-yl)phenyl)pyrido[4,3-d]pyrimidin-2-amine;
8-(cyclopent-1-en-1-yl)-N-(2-methoxy-4-(4-methyl pip erazin-1-yl)phenyl)pyrido[4,3-d]pyrimidin-2-amine;
6-acetyl-8-cyclopentylethoxy-4-(4-methylpiperazin-1-yl)phenyl)amino)-5-methylpyrido[2,3-d]pyrimidin-7(8H)-one;
6-acetyl-8-cyclopentylethoxy-4-(piperazin-1-yl)phenyl)amino)-5-methylpyrido[2,3-d]pyrimidin-7(8H)-one;
8-cyclohexyl-2-((2-methoxy-4-(4-methylpiperazin-1-yl)phenyl)amino)-6-methylpyrido[2,3-d]pyrimidin-7(8H)-one;
8-cyclobutyl -2-((2-methoxy-4-(4-methylpiperazin-1-yl)phenyl)amino)-6-methylpyrido[2,3-d]pyrimidin-7(8H)-one;
8-cycloheptylethoxy-4-(4-methylpiperazin-1-yl)phenyl)amino)-6-methylpyrido[2,3-d]pyrimidin-7(8H)-one; and
8-cycloheptyl ethoxy-4-(4-methylpiperazin-1-yl)phenyl)amino)pyrido[2,3-d]pyrimidin-7(8H)-one.

8.-9. (canceled)

10. A pharmaceutical composition comprising the compound of claim 1, or pharmaceutically acceptable salts thereof, or geometric isomers, enantiomers, diastereomers, racemates, isotope-substituted derivatives, solvates, hydrates or prodrugs thereof and a pharmaceutically acceptable carrier.

11. The compound of claim 2, or pharmaceutically acceptable salts thereof, or geometric isomers, enantiomers, diastereomers, racemates, isotope-substituted derivatives, solvates, hydrates or prodrugs thereof, wherein:
(1) R₀ is optionally substituted C₃-C₇ cycloalkyl, optionally substituted C₃-C₇ cycloalkenyl or optionally substituted 3-7 membered heterocyclic group;
(2) B₁ is N or CR₁, B₂ is N, B₃ is N or CR₃, R₃ is H, halogen or C₁-C₄ alkyl, R₃ is H, halogen or C₁-C₄ alkyl; or B₁ is N, B₂ is N, B₃ is CR₃, wherein R₃ is H or C₁-C₄ alkyl; or B₁ is N, B₂ is N, and B₃ is CH;
(3) D₁, D₂ and D₃ are each independently N or CH; or D₁ is CR₄, D₂ is CR₅, and D₃ is CR₆; R₄, R₅ and R₆ are each independently H, halogen and C₁-C₄ alkyl; or all of D₁, D₂ and D₃ are CH;
(4) R₇ is selected from halogen, optionally substituted C₁-C₃ alkyl and optionally substituted C₁-C₃ alkoxy;
(5) R₈ is selected from optionally substituted C₁-C₆ alkyl, optionally substituted C₁-C₆ alkoxy, optionally substituted C₁-C₆ alkylamino and optionally substituted 4-7 membered nitrogen or oxygen-containing heterocyclic group.

12. The compound of claim 2, or pharmaceutically acceptable salts thereof, or geometric isomers, enantiomers, diastereomers, racemates, isotope-substituted derivatives, solvates, hydrates or prodrugs thereof, wherein the fused bicyclic ring formed by ring A and the ring containing B₁-B₃ is selected from: 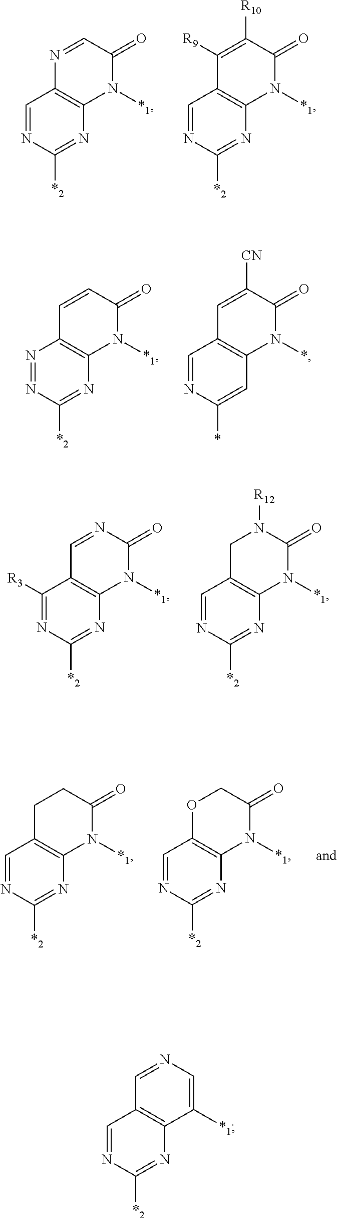
wherein, *₁ and *₂ refer to the attachment positions of the said groups to R₀ and —NH in the rest of the compound; R₃ is H or C₁-C₃ alkyl; R₉ is H or C₁-C₃ alkyl; R₁₀ is H, C₁-C₃ alkyl, C₂-C₄ acyl or cyano; R₁₂ is H or C₁-C₃ alkyl.

13. The compound of claim 4, or pharmaceutically acceptable salts thereof, or geometric isomers, enantiomers, diastereomers, racemates, isotope-substituted derivatives, solvates, hydrates or prodrugs thereof, wherein:
R₀ is C₃-C₇ cycloalkyl, C₃-C₇ cycloalkenyl or C₃-C₇ heterocyclic group optionally substituted by 1, 2 or 3 groups selected from the group consisting of halogen, C₁-C₆ alkyl, halogenated C₁-C₆ alkyl, hydroxyl, C₁-C₆ alkoxy, halogenated C₁-C₆ alkoxy and —NRₐR_{b}, wherein Rₐ and R_{b} are each independently H or C₁-C₄ alkyl; and/or
B₁ is N or CR₁, B₂ is N, B₃ is N or CR₃, wherein R₁ is H, halogen or C₁-C₄ alkyl, R₃ is H, halogen or C₁-C₄ alkyl; or B₁, B₂ and B₃ are each independently N or CH; or B₁ is N, B₂ is N, and B₃ is CR₃, wherein R₃ is H or C₁-C₄ alkyl; or B₁ is N, B₂ is N, and B₃ is CH; and/or
D₁, D₂ and D₃ are each independently N or CH; or D₁ is CR₄, D₂ is CR₅, and D₃ is CR₆, wherein R₄, R₅ and R₆ are each independently H, halogen and C₁-C₄ alkyl; or all of D₁, D₂ and D₃ are CH; and/or
R₇ is halogen, C₁-C₃ alkyl or C₁-C₃ alkoxy optionally substituted by 1, 2, 3, 4 or 5 groups independently selected from a group consisting of halogen, hydroxyl and —NRₐR_{b}, wherein Rₐ and R_{b} are each independently H or C₁-C₄ alkyl; and/or
R₈ is C₁-C₆ alkyl, C₁-C₆ alkoxy, C₁-C₆ alkylamino or heterocyclic group optionally substituted by 1-4 groups selected from the group consisting of C₁-C₆ alkyl, C₁-C₆ alkyl substituted with hydroxy, halogenated C₁-C₆ alkyl, C₁-C₆ alkoxy, C₁-C₆ acyl, heterocyclic group optionally substituted with 1-4 C₁-C₆ alkyl, halogen, —NRₐR_{b} and hydroxyl, wherein Rₐ and R_{b} are each independently H or C₁-C₆ alkyl; or R₈ is connected with D₁ or D₂ to form a 4-7 membered heterocyclic group or a 5-14 membered heteroaryl optionally substituted by 1, 2 or 3 groups selected from the group consisting of C₁-C₆ alkyl, C₁-C₆ acyl, heterocyclic group optionally substituted with 1-4 C₁-C₆ alkyl, halogen, —NR a R_{b} and hydroxyl, wherein Rₐ and R_{b} are each independently H or C₁-C₆ alkyl; and/or
R₉ is H or C₁-C₃ alkyl; and/or
R₁₀ is H, C₁-C₃ alkyl, cyano or C₂-C₄ acyl; and/or
A₃ is selected from 0 and CR₁₃R′₁₃, A₄ is selected from O, NR₁₂ and CR₁₄R′₁₄, wherein R₁₃, R′₁₃, R₁₄ and R′₁₄ are each independently H or C₁-C₃ alkyl; and/or
A₁ and A₂ are each independently N, CH, C—CN, C-(C₁-C₃ alkyl) or C-(C₂-C₄ acyl).

14. The compound of claim 4, or pharmaceutically acceptable salts thereof, or geometric isomers, enantiomers, diastereomers, racemates, isotope-substituted derivatives, solvates, hydrates or prodrugs thereof, wherein:
in Formula IIa, the fused bicyclic ring containing A₁, A₂, B₁, B₂ and B₃ is selected from: 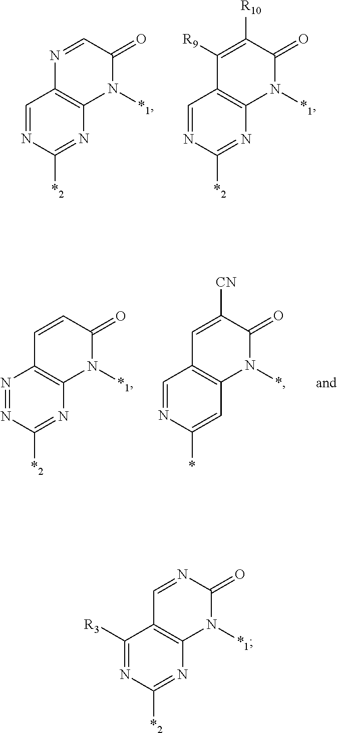
wherein *₁ and *₂ refer to an attachment position of the said groups to R₀ and —NH in the rest of the compound; R₃ is H or C₁-C₃ alkyl; R₉ is H or C₁-C₃ alkyl; R₁₀ is H, C₁-C₃ alkyl, cyano or C₂-C₄ acyl;
in Formula IIb, the fused bicyclic ring containing A₃, A₄, B₁, B₂ and B₃ is selected from: 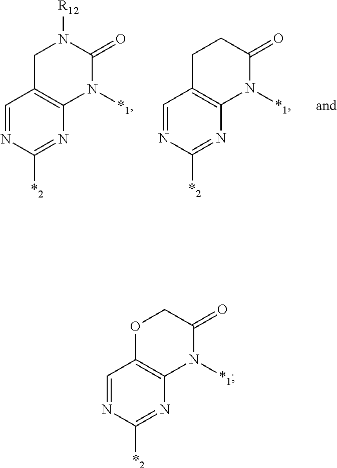
wherein *₁ and *₂ refer to an attachment position of the said groups to R₀ and —NH in the rest of the compound; R₁₂ is H or C₁-C₃ alkyl;
in Formula IIc, the fused bicyclic ring containing A₁, A₂, B₁, B₂ and B₃ is: 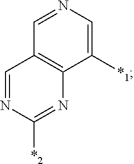
wherein *₁ and *₂ refer to an attachment position of the said groups to R₀ and —NH in the rest of the compound.

15. The compound of claim 6, or pharmaceutically acceptable salts thereof, or geometric isomers, enantiomers, diastereomers, racemates, isotope-substituted derivatives, solvates, hydrates or prodrugs thereof, wherein:
R₀ is C₃-C₇ cycloalkyl, C₃-C₇ cycloalkenyl or C₃-C₇ heterocyclic group optionally substituted by 1, 2 or 3 groups selected from the group consisting of halogen, C₁-C₆ alkyl, halogenated C₁-C₆ alkyl, hydroxyl, C₁-C₆ alkoxy, halogenated C₁-C₆ alkoxy and —NRₐR_{b}, wherein Rₐ and R_{b} are each independently H or C₁-C₄ alkyl; and/or
B₁ is N or CR₁, B₂ is N, B₃ is N or CR₃, wherein R₁ is H, halogen or C₁-C₄ alkyl, R₃ is H, halogen or C₁-C₄ alkyl; or B₁, B₂ and B₃ are each independently N or CH; or B₁ is N, B₂ is N, and B₃ is CR₃, wherein R₃ is H or C₁-C₄ alkyl; or B₁ is N, B₂ is N, and B₃ is CH; and/or
R₇ is halogen, C₁-C₃ alkyl or C₁-C₃ alkoxy optionally substituted by 1, 2, 3, 4 or 5 groups independently selected from the group consisting of halogen, hydroxyl and —NRₐR_{b}, wherein Rₐ and R_{b} are each independently H or C₁-C₄ alkyl; and/or
R₉ is H or C₁-C₃ alkyl; and/or
R₁₀ is H, C₁-C₃ alkyl, cyano or C₂-C₄ acyl; and/or
Cy is piperazinyl, piperidinyl, morpholinyl or 1,4-diazepan-1-yl optionally substituted by 1, 2 or 3 groups selected from the group consisting of C₁-C₆ alkyl, C₁-C₆ alkyl substituted with hydroxy, halogenated C₁-C₆ alkyl, C₁-C₆ alkoxy, C₁-C₆ acyl, heterocyclic group optionally substituted with 1-4 C₁-C₆ alkyl, halogen, —NRₐR_{b} and hydroxyl, wherein Rₐ and R_{b} are each independently H or C₁-C₆ alkyl; or Cy is piperazinyl optionally substituted by 1-3 substituents selected from C₁-C₆ alkyl and C₁-C₆ alkyl substituted with hydroxyl, piperidinyl optionally substituted by 1 substituent selected from C₁-C₆ alkyl and —NRₐR_{b}, 1,4-diazepan-1-yl optionally substituted by 1-3 C₁-C₆ alkyl, or morpholinyl optionally substituted by 1-3 C₁-C₆ alkyl; wherein Rₐ and R_{b} are each independently H or C₁-C₄ alkyl.

16. A method for treatment and prevention of NUAK1/2-mediated diseases, disorders and conditions, comprising administering to a subject in need thereof the compound of claim 1 or a pharmaceutically acceptable salt thereof, or geometric isomer, enantiomer, diastereomer, racemate, isotope-substituted derivative, solvate, hydrate or prodrug thereof, or a pharmaceutical composition containing the compound of claim 1 or a pharmaceutically acceptable salt thereof, or geometric isomer, enantiomer, diastereomer, racemate, isotope-substituted derivative, solvate, hydrate or prodrug thereof and a pharmaceutically acceptable carrier.

17. The method according to claim 16, wherein the disease or condition is a cancer selected from liver cancer, melanoma, Hodgkin's disease, non-Hodgkin's lymphomas, acute lymphocytic leukemia, chronic lymphocytic leukemia, multiple myeloma, neuroblastoma, breast cancer, ovarian cancer, Wilms' tumor, cervical cancer, testicular cancer, soft-tissue sarcoma, primary macroglobulinemia, bladder cancer, chronic myeloid leukemia, primary brain cancer, malignant melanoma, small cell lung cancer, lung cancer, gastric cancer, colon cancer, malignant pancreatic islet tumor, malignant carcinoid cancer, choriocarcinoma, mycosis fungoide, head and neck cancer, osteogenic sarcoma, pancreatic cancer, acute myeloid leukemia, hairy cell leukemia, rhabdomyosarcoma, Kaposi's sarcoma, urogenital tumors, thyroid cancer, esophageal cancer, malignant hypercalcemia, cervical hyperplasia, renal cell carcinoma, endometrial cancer, polycythemia vera, idiopathic thrombocythemia, adrenocortical carcinoma, skin cancer, and prostatic cancer; or the method further is used in combination with radiotherapy.

18. The method according to claim 17, wherein the method further comprises administering to the subject at least one known anticancer drug or a pharmaceutically acceptable salt thereof selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, T-DM1, Trastuzumab Deruxtecan, Trastuzumab Emtansine, Datopotamab Deruxtecan, Gemtuzumab Ozogamicin, Brentuximab Vedotin, Inotuzumab Ozogamicin, Sacituzumab govitecan, Enfortumab Vedotin, Belantamab Mafodotin, imatinib, gefitinib, erlotinib, osimertinib, afatinib, ceritinib, alectinib, crizotinib, erlotinib, lafenib, sorafenib, regorafenib, vemurafenib, dabrafenib, aflibercept, sunitinib, nilotinib, dasatinib, bosutinib, ponatinib, ibrutinib, cabozantinib, lenvatinib, vandetanib, trametinib, cobimetinib, axitinib, temsirolimus, idelalisib, pazopanib, Torisel, everolimus, tamoxifen, letrozole, fulvestrant, mitoguazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin, sipueucel-T, palbociclib, olaparib, niraparib, rucaparib, talazoparib, pamiparib, fluzoparib and senaparib.

19. The method according to claim 16, wherein the compound is selected from the group consisting of:
8-cyclopentyl-2-((2-methoxy-4-(4-methylpiperazin-1-yl)phenyl)amino)pteridin-7(8H)-one;
1-cyclopentyl-7-((2-methoxy-4-(4-methylpiperazin-1-yl)phenyl)amino)-3,4-dihydropyrimido[4,5-d]pyrimidin-2(1H)-one;
1-cyclopropyl-7-((2-methoxy-4-(4-methylpiperazin-1-yl)phenyl)amino)-3,4-dihydropyrimido[4,5-d]pyrimidin-2(1H)-one;
1-cyclopentyl-7-((2-methoxy-4-(4-methylpiperazin-1-yl)phenyl)amino)-3-methyl-3,4-dihydropyrimido[4,5-d]pyrimidin-2(1H)-one;
1-cyclopentyl-7-((2-methoxy-4-(4-methylpiperazin-1-yl)phenyl)amino)pyrimido[4,5-d]pyrimidin-2(1H)-one;
1-cyclohexyl-7-((2-methoxy-4-(4-methylpiperazin-1-yl)phenyl)amino)pyrimido[4,5-d]pyrimidin-2(1H)-one;
1-cyclopentyl-7-((2-ethoxy-4-(4-methylpiperazin-1-yl)phenyl)amino)pyrimido[4,5-d]pyrimidin-2(1H)-one;
1-cyclopentyl-7-((2-isopropoxy-4-(4-methylpiperazin-1-yl)phenyl)amino)pyrimido[4,5-d]pyrimidin-2(1H)-one;
1-cyclopentyl-7-((4-(4-methylpiperazin-1-yl)-2-(trifluoromethoxy)phenyl)amino)pyrimido[4,5-d]pyrimidin-2(1H)-one;
1-cyclopentyl-7-((2-methyl-4-(4-methylpiperazin-1-yl)phenyl)amino)pyrimido[4,5-d]pyrimidin-2(1H)-one;
7-((2-chloro-4-(4-methylpiperazin-1-yl)phenyl)amino)-1-cyclopentylpyrimido[4,5-d]pyrimidin-2(1H)-one;
7-((2-bromo-4-(4-methylpiperazin-1-yl)phenyl)amino)-1-cyclopentylpyrimido[4,5-d]pyrimidin-2(1H)-one;
1-cyclopentyl-7-((2-methoxy-4-(4-methylpiperazin-1-yl)phenyl)amino)-5-methylpyrimido[4,5-d]pyrimidin-2(1H)-one;
1-cyclobutyl-7-((2-methoxy-4-(4-methylpiperazin-1-yl)phenyl)amino)pyrimido[4,5-d]pyrimidin-2(1H)-one;
1-cyclopropyl-7-((2-methoxy-4-(4-methylpiperazin-1-yl)phenyl)amino)pyrimido[4,5-d]pyrimidin-2(1H)-one;
7-((2-methoxy-4-(4-methylpiperazin-1-yl)phenyl)amino)-1-(tetrahydro-2H-pyran-4-yl)pyrimido[4,5-d]pyrimidin-2(1H)-one;
7-((2-methoxy-4-(4-methylpiperazin-1-yl)phenyl)amino)-1-(piperidin-4-yl)pyrimido[4,5-d]pyrimidin-2(1H)-one;
7-((2-methoxy-4-(4-methylpiperazin-1-yl)phenyl)amino)-1-(1-methylpiperidin-4-yl)pyrimido[4,5-d]pyrimidin-2(1H)-one;
8-cyclopentyl-2-((2-methoxy-4-(4-methylpiperazin-1-yl)phenyl)amino)pyrido[2,3-d]pyrimidin-7(8H)-one;
8-cyclopentyl-2-((2-methoxy-4-(4-methylpiperazin-1-yl)phenyl)amino)-6-methylpyrido[2,3-d]pyrimidin-7(8H)-one;
8-cyclopropyl-2-(2-methoxy-4-(4-methylpiperazin-1-yl)phenyl)amino)-6-methylpyrido[2,3-d]pyrimidin-7(8H)-one;
8-cyclohexyl-2-methoxy-4-(4-methylpiperazin-1-yl)phenyl)amino)pyrido[2,3-d]pyrimidin-7(8H)-one;
8-cyclopropyl-2-(2-methoxy-4-(4-methylpiperazin-1-yl)phenyl)amino)pyrido[2,3-d]pyrimidin-7(8H)-one;
5-cyclopentyl-3-((2-methoxy-4-(4-methylpiperazin-1-yl)phenyl)amino)pyrido[2,3-e][1,2,4]triazin-6(5H)-one;
1-cyclopentyl -7-((2-methoxy-4-(4-methylpiperazin-1-yl)phenyl)amino)-2-oxo-1,2-dihydro-1,6-naphthyri dine-3-carbonitrile;
8-cyclopentyl-2-((2-methoxy-4-(4-methylpiperazin-1-yl)phenyl)amino)-7-oxo-7,8-dihydropyrido[2,3-d]pyrimidine-6-carbonitrile;
8-cyclopentyl -2-((2-methoxy-4-(4-methylpiperazin-1-yl)phenyl)amino)-5-methylpyrido[2,3-d]pyrimidin-7(8H)-one;
8-cyclopentyl -2-((2-methoxy-4-(4-methylpiperazin-1-yl)phenyl)amino)-5,8-dihydropyri do [2,3-d]pyrimidin-7(6H)-one;
8-cyclohexyl -2-((2-methoxy-4-(4-methylpiperazin-1-yl)phenyl)amino)-5,8-dihydropyrido[2,3-d]pyrimidin-7(6H)-one;
8-cyclopropyl-2-(2-methoxy-4-(4-methylpiperazin-1-yl)phenyl)amino)-5,8-dihydropyri do [2,3-d]pyrimidin-7(6H)-one;
8-cyclopentyl -2-((2-methoxy-4-(4-methylpiperazin-1-yl)phenyl)amino)-6H-pyrimido[5,4-b] [1,4]oxazin-7(8H)-one;
7-((2-bromo-4-(4-methylpiperazin- I -yl)phenyl)amino)-1-cyclopentyl-3,4-dihydropyrimido[4,5-d]pyrimidin-2(1H)-one;
7-((2-methoxy-4-(4-methylpiperazin-1-yl)phenyl)amino)-1-(tetrahydro-2H-pyran-4-yl)-3,4-dihydropyrimido[4,5-d]pyrimidin-2(1H)-one;
8-cyclopentyl -N-(2-methoxy-4-(4-methylpiperazin-1-yl)phenyl)pyrido[4,3-d]pyrimidin-2-amine;
8-(cycl opent-1-en-1-yl)-N-(2-methoxy-4-(4-methyl piperazin-1-yl)phenyl)pyrido[4,3-d]pyrimidin-2-amine;
6-acetyl-8-cyclopentyl-2-((2-methoxy-4-(4-methylpiperazin-1-yl)phenyl)amino)-5-methylpyrido[2,3-d]pyrimidin-7(8H)-one;
6-acetyl-8-cyclopentyl -2-((2-methoxy-4-(piperazin-1-yl)phenyl)amino)-5-methylpyrido[2,3-d]pyrimidin-7(8H)-one;
8-cyclohexyl-2-((2-methoxy-4-(4-methylpiperazin-1-yl)phenyl)amino)-6-methylpyrido[2,3-d]pyrimidin-7(8H)-one;
8-cyclobutyl-2-((2-methoxy-4-(4-methylpiperazin-1-yl)phenyl)amino)-6-methylpyrido[2,3-d]pyrimidin-7(8H)-one;
8-cycloheptyl-2-((2-methoxy-4-(4-methylpiperazin-1-yl)phenyl)amino)-6-methylpyrido[2,3-d]pyrimidin-7(8H)-one; and
8-cycloheptyl-2-((2-methoxy-4-(4-methylpiperazin-1-yl)phenyl)amino)pyrido[2,3-d]pyrimidin-7(8H)-one.

20. The pharmaceutical composition according to claim 10, wherein the compound is selected from the group consisting of:
8-cyclopentyl-2-((2-methoxy-4-(4-methylpiperazin-1-yl)phenyl)amino)pteridin-7(8H)-one;
1-cyclopentyl-7-((2-methoxy-4-(4-methylpiperazin-1-yl)phenyl)amino)-3,4-dihydropyrimido[4,5-d]pyrimidin-2(1H)-one;
1-cyclopropyl-7-((2-methoxy-4-(4-methylpiperazin-1-yl)phenyl)amino)-3,4-dihydropyrimido[4,5-d]pyrimidin-2(1H)-one;
1-cyclopentyl-7-((2-methoxy-4-(4-methylpiperazin-1-yl)phenyl)amino)-3-methyl-3,4-dihydropyrimido[4,5-d]pyrimidin-2(1H)-one;
1-cyclopentyl-7-((2-methoxy-4-(4-methylpiperazin-1-yl)phenyl)amino)pyrimido[4,5-d]pyrimidin-2(1H)-one;
1-cyclohexyl-7-((2-methoxy-4-(4-methylpiperazin-1-yl)phenyl)amino)pyrimido[4,5-d]pyrimidin-2(1H)-one;
1-cyclopentyl-7-((2-ethoxy-4-(4-methylpiperazin-1-yl)phenyl)amino)pyrimido[4,5-d]pyrimidin-2(1H)-one;
1-cyclopentyl-7-((2-isopropoxy-4-(4-methylpiperazin-1-yl)phenyl)amino)pyrimido[4,5-d]pyrimidin-2(1H)-one;
1-cyclopentyl-7-((4-(4-methylpiperazin-1-yl)-2-(trifluoromethoxy)phenyl)amino)pyrimido[4,5-d]pyrimidin-2(1H)-one;
1-cyclopentyl-7-((2-methyl-4-(4-methylpiperazin-1-yl)phenyl)amino)pyrimido[4,5-d]pyrimidin-2(1H)-one;
7-((2-chloro-4-(4-methylpiperazin-1-yl)phenyl)amino)-1-cyclopentylpyrimido[4,5-d]pyrimidin-2(1H)-one;
7-((2-bromo-4-(4-methylpiperazin-1-yl)phenyl)amino)-1-cyclopentylpyrimido[4,5-d]pyrimidin-2(1H)-one;
1-cyclopentyl-7-((2-methoxy-4-(4-methylpiperazin-1-yl)phenyl)amino)-5-methylpyrimido[4,5-d]pyrimidin-2(1H)-one;
1-cyclobutyl-7-((2-methoxy-4-(4-methylpiperazin-1-yl)phenyl)amino)pyrimido[4,5-d]pyrimidin-2(1H)-one;
1-cyclopropyl-7-((2-methoxy-4-(4-methylpiperazin-1-yl)phenyl)amino)pyrimido[4,5-d]pyrimidin-2(1H)-one;
7-((2-methoxy-4-(4-methylpiperazin-1-yl)phenyl)amino)-1-(tetrahydro-2H-pyran-4-yl)pyrimido[4,5-d]pyrimidin-2(1H)-one;
7-((2-methoxy-4-(4-methylpiperazin-1-yl)phenyl)amino)-1-(piperidin-4-yl)pyrimido[4,5-d]pyrimidin-2(1H)-one;
7-((2-methoxy-4-(4-methylpiperazin-1-yl)phenyl)amino)-1-(1-methylpiperidin-4-yl)pyrimido[4,5-d]pyrimidin-2(1H)-one;
8-cyclopentyl-2-((2-methoxy-4-(4-methylpiperazin-1-yl)phenyl)amino)pyrido[2,3-d]pyrimidin-7(8H)-one;
8-cyclopentyl-2-((2-methoxy-4-(4-methylpiperazin-1-yl)phenyl)amino)-6-methylpyrido[2,3-d]pyrimidin-7(8H)-one;
8-cyclopropyl-2-(2-methoxy-4-(4-methylpiperazin-1-yl)phenyl)amino)-6-methylpyrido[2,3-d]pyrimidin-7(8H)-one;
8-cyclohexyl-2-((2-methoxy-4-(4-methylpiperazin-1-yl)phenyl)amino)pyrido[2,3-d]pyrimidin-7(8H)-one;
8-cyclopropyl-2-(2-methoxy-4-(4-methylpiperazin-1-yl)phenyl)amino)pyrido[2,3-d]pyrimidin-7(8H)-one;
5-cyclopentyl-3-((2-methoxy-4-(4-methylpiperazin-1-yl)phenyl)amino)pyrido[2,3-e][1,2,4]triazin-6(5H)-one;
1-cyclopentyl -7-((2-methoxy-4-(4-methylpiperazin-1-yl)phenyl)amino)-2-oxo-1,2-dihydro-1,6-naphthyridine-3-carbonitrile;
8-cyclopentyl-2-((2-methoxy-4-(4-methylpiperazin-1-yl)phenyl)amino)-7-oxo-7,8-dihydropyrido[2,3-d]pyrimidine-6-carbonitrile;
8-cyclopentyl-2-((2-methoxy-4-(4-methylpiperazin-1-yl)phenyl)amino)-5-methylpyrido[2,3-d]pyrimidin-7(8H)-one;
8-cyclopentyl -2-((2-methoxy-4-(4-methylpiperazin-1-yl)phenyl)amino)-5,8-dihydropyrido[2,3-d]pyrimidin-7(6H)-one;
8-cyclohexyl -2-((2-methoxy-4-(4-methylpiperazin-1-yl)phenyl)amino)-5,8-dihydropyrido[2,3-d]pyrimidin-7(6H)-one;
8-cyclopropyl-2-(2-methoxy-4-(4-methylpiperazin-1-yl)phenyl)amino)-5,8-dihydropyrido[2,3-d]pyrimidin-7(6H)-one;
8-cyclopentyl -2-((2-methoxy-4-(4-methylpiperazin-1-yl)phenyl)amino)-6H-pyrimido[5,4-b][1,4]oxazin-7(8H)-one;
7-((2-bromo-4-(4-methylpiperazin-1-yl)phenyl)amino)-1-cyclopentyl-3,4-dihydropyrimido[4,5-d]pyrimidin-2(1H)-one;
7-((2-methoxy-4-(4-methylpiperazin-1-yl)phenyl)amino)-1-(tetrahydro-2H-pyran-4-yl)-3,4-dihydropyrimido[4,5-d]pyrimidin-2(1H)-one;
8-cyclopentyl-N-(2-methoxy-4-(4-methylpiperazin-1-yl)phenyl)pyrido[4,3-d]pyrimidin-2-amine;
8-(cyclopent-1-en-1-yl)-N-(2-methoxy-4-(4-methylpiperazin-1-yl)phenyl)pyrido[4,3-d]pyrimidin-2-amine;
6-acetyl-8-cyclopentyl-2-((2-methoxy-4-(4-methylpiperazin-1-yl)phenyl)amino)-5-methylpyrido[2,3-d]pyrimidin-7(8H)-one;
6-acetyl-8-cyclopentyl -2-((2-methoxy-4-(piperazin-1-yl)phenyl)amino)-5-methylpyrido[2,3-d]pyrimidin-7(8H)-one;
8-cyclohexyl-2-((2-methoxy-4-(4-methylpiperazin-1-yl)phenyl)amino)-6-methylpyrido[2,3-d]pyrimidin-7(8H)-one;
8-cyclobutyl-2-((2-methoxy-4-(4-methylpiperazin-1-yl)phenyl)amino)-6-methylpyrido[2,3-d]pyrimidin-7(8H)-one;
8-cycloheptyl-2-((2-methoxy-4-(4-methylpiperazin-1-yl)phenyl)amino)-6-methylpyrido[2,3-d]pyrimidin-7(8H)-one; and
8-cycloheptyl-2-((2-methoxy-4-(4-methylpiperazin-1-yl)phenyl)amino)pyrido[2,3-d]pyrimidin-7(8H)-one.

21. The pharmaceutical composition according to claim 10, wherein the composition further includes at least one known anticancer drug selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, methylhydroxy ellipticine, etoposide, 5-azacytidine, gemcitabine, 5-fluorouracil, capecitabine, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, mAb, panitumumab, necitumumab, nivolumab, pembrolizumab, ramucirumab, bevacizumab, pertuzumab, trastuzumab, cetuximab, obinutuzumab, ofatumumab, rituximab, alemtuzumab, ibritumomab, tositumomab, brentuximab, daratumumab, elotuzumab, T-DM1, Ofatumumab, Dinutuximab, Blinatumomab, ipilimumab, avastin, herceptin, mabthera, imatinib, gefitinib, erlotinib, ostinib, afatinib, ceritinib, alectinib, crizotinib, erlotinib, lapatinib, solutinib lafenib, regorafenib, vemurafenib, dabrafenib, aflibercept, sunitinib, nilotinib, dasatinib, bosutinib, pratinib, brutinib, cabozantinib, lenvatinib, vandetanib, trametinib, cabitinib, axitinib, temsirolimus, idelalisib, pazopanib, everolimus, tamoxifen, letrozole, fulvestrant, mitoguanhydrazone, octreotide, retinoic acid, arsenic, zoledronic acid, bortezomib, carfilzomib, Ixazomib, vismodegib, sonidegib, denosumab, thalidomide, lenalidomide, Venetoclax, Aldesleukin, sipueucel-T, palbociclib, olaparib, niraparib, rucaparib, talazoparib, pamiparib, fluzoparib and senaparib.
